SEQUENCE LISTING (85 sequences)

SEQ ID NO: 1 - DNA, 1704 bp, Influenza A
atggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagacacacaacgggaagctctgcgatctagatggagtgaagcctctaattttgagagattgtagtgtagctggatggctcctcggaaacccaatgtgtgacgaattcatcaatgtgccggaatggtcctacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagagaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagtatgccattccacaatatacaccctctcaccatcggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggaactgggtaacggttgtttcgagttctatcataaatgtgataatgaatgtatggaaagtgtaagaaacggaacgtatgactacccgcagtattcagaagaagcaagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatt

SEQ ID NO: 2 - protein, 568 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 3 - protein, 11 aa, Influenza A
PQRERRRKKRG

SEQ ID NO: 4 - protein, 7 aa, Influenza A
PQRETRG

SEQ ID NO: 5 - protein, 43 aa, Influenza A
LVPRGSPGSGYIPEAPRDGQAYVRKDGEWVLLSTFLGHHHHHH

SEQ ID NO: 6 - protein, 47 aa, Influenza A
ETTKGVTAACSYAPPTGTDQQSLYQNADAYIAARPKVRDQAGRMNYY

SEQ ID NO: 7 - protein, 47 aa, Influenza A
EASLGVSSACPYQPNDAAEQTKLYQNPTTYIATRSKVNGQSGRMEFF

SEQ ID NO: 8 - protein, 568 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAADQTSLYQNPTTYISVGTSTLNQRLVPRIATRSKVNDQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 9 - protein, 564 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAADQTSLYQNPTTYISVGTSTLNQRLVPRIATRSKVNDQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 10 - protein, 561 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAADQTSLYQNPTTYISVGTSTLNQRLVPRIATRSKVNDQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGRLVPRGSPGSGYIPEAPRDGQAYVRKDGEWVLLSTFLGHHHHHH

SEQ ID NO: 11 - protein, 568 aa, Influenza A
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAADQTSLYQNPTTYISIGTSTLNQRLVPKIATRSKVNDQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 12 - protein, 564 aa, Influenza A
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAADQTSLYQNPTTYISIGTSTLNQRLVPKIATRSKVNDQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 13 - protein, 561 aa, Influenza A
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAADQTSLYQNPTTYISIGTSTLNQRLVPKIATRSKVNDQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGRLVPRGSPGSGYIPEAPRDGQAYVRKDGEWVLLSTFLGHHHHHH

SEQ ID NO: 14 - DNA, 6110 bp, Influenza A
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgagtcaccgtcgtcgacacgatccgatatcgccgccaccatggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcagcgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgatcagacaagcctgtaccagaatcctacaacatacatcagcgtgggaacaagcacactgaatcagagactggtgcctagaatcgccacaagaagcaaggtgaatggacagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagagaagaagaaagaagagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctgagcggccgctctagaccaggccctggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 6124 bp, Influenza A
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgatccgatatcgccgccaccatggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcagcgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagacaagcctgtaccagaatcctacaacatacatcagcgtgggaacaagcacactgaatcagagactggtgcctagaatcgccacaagaagcaaggtgaatggactgagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagagaagaagaaagaagagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctgagcggccgctctagaccaggccctggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 6124 bp, Influenza A
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacgggaacttccatagcccatatatggagttccgcgttacataacttacgggaatttccaaacctggctgaccgcccaacgacccccgcccattgacgtcaataatgacgtatgttcccatagtaacgccaatagggaacttccattgacgtcaatgggtggagtatttacggtaaactgcccacttgggaatttccaagtgtatcatatgccaagtacgccccctattgacgtcaatgacgggaacttccataagcttgcattatgcccagtacatgaccttatgggaatttcctacttggcagtacatctacgtattagtcatcgctattaccatggtgatgcggttttggcagtacatcaatgggcgtggatagcggtttgactcacgggaacttccaagtctccaccccattgacgtcaatgggagtttgttttgactcaccaaaatcaacgggaattcccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgatccgatatcgccgccaccatggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcagcgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagacaagcctgtaccagaatcctacaacatacatcagcgtgggaacaagcacactgaatcagagactggtgcctagaatcgccacaagaagcaaggtgaatggactgagctccagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagagaagaagaaagaagagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctgagcggccgctctagaccaggccctggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - protein, 568 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSAACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 18 - protein, 564 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSAACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 19 - protein, 561 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSAACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGRLVPRGSPGSGYIPEAPRDGQAYVRKDGEWVLLSTFLGHHHHHH

SEQ ID NO: 20 - protein, 568 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAAEQIKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 21 - protein, 564 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAAEQIKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 22 - protein, 561 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAAEQIKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGRLVPRGSPGSGYIPEAPRDGQAYVRKDGEWVLLSTFLGHHHHHH

SEQ ID NO: 23 - protein, 568 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSAACPYQRKSSFFRNVVWLIKKNSTYPTIKRSYNNTNQEDLLVLWGIHHPNDAAEQIKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 24 - protein, 564 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSAACPYQRKSSFFRNVVWLIKKNSTYPTIKRSYNNTNQEDLLVLWGIHHPNDAAEQIKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 25 - protein, 561 aa, Influenza A
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSAACPYQRKSSFFRNVVWLIKKNSTYPTIKRSYNNTNQEDLLVLWGIHHPNDAAEQIKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGRLVPRGSPGSGYIPEAPRDGQAYVRKDGEWVLLSTFLGHHHHHH

SEQ ID NO: 26 - DNA, 1707 bp, Influenza A
atggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcagcgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgatcagacaagcctgtaccagaatcctacaacatacatcagcgtgggaacaagcacactgaatcagagactggtgcctagaatcgccacaagaagcaaggtgaatgatcagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagagaagaagaaagaagagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctga

SEQ ID NO: 27 - DNA, 1695 bp, Influenza A
atggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcagcgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgatcagacaagcctgtaccagaatcctacaacatacatcagcgtgggaacaagcacactgaatcagagactggtgcctagaatcgccacaagaagcaaggtgaatgatcagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagacgagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctga

SEQ ID NO: 28 - DNA, 1686 bp, Influenza A
atggagaagattgtgctgctgttcgccattgtgagcctggtgaagagcgatcagatctgtattggctaccacgccaacaattctacagagcaggtggacaccatcatggagaaaaacgtgacagtgacacacgctcaggacatcctggagaaaacccacaatggcaagctgtgtgatctggatggagtgaagcctctgatcctgagagattgttctgtggctggatggctgctgggaaaccctatgtgtgacgagttcatcaatgtgcctgagtggagctatatcgtggagaaggccaaccctgtgaatgatctgtgttaccccggcgacttcaatgattacgaggagctgaagcacctgctgtccagaatcaaccacttcgagaagatccagatcatccctaagtctagctggtctagccatgaagcttctctgggagtgtctagcgcttgtccctatcagagaaagagcagcttcttcagaaatgtggtgtggctgatcaagaagaacagcacctaccccacaatcaagcggagctacaacaacaccaaccaggaagatctgctggtcctgtggggaattcaccatcctaatgatgccgccgatcagacatctctgtaccagaaccccaccacatatatctctgtgggcaccagcacactgaatcagagactggtgcctagaatcgccacaagatccaaggtgaacgatcagtctggcagaatggagttcttctggaccatcctgaagccaaacgacgccatcaacttcgagagcaacggcaatttcatcgcccctgagtacgcctataagatcgtgaagaagggcgatagcaccatcatgaagagcgagctggagtacggcaactgtaataccaagtgccagacacctatgggcgccatcaatagctctatgcccttccacaatatccaccctctgacaatcggcgagtgtcctaagtacgtgaagagcaacagactggtgctggctacaggcctgagaaatagccctcagagagagacaagaggactgtttggagccatcgccggattcattgaagggggatggcagggaatggtcgatggctggtatggctatcaccacagcaatgagcagggatctggatatgccgccgataaggagtctacacagaaggccatcgacggcgtcacaaacaaggtgaacagcatcatcgacaagatgaacacccagtttgaggctgtgggcagagagttcaacaacctggagcggagaatcgagaacctgaacaagaagatggaggacggctttctggatgtgtggacctataatgccgaactgctggtgctgatggagaacgagagaaccctggatttccacgacagcaacgtgaagaacctgtacgacaaagtgagactgcagctgagagataatgccaaggaactgggcaatggctgcttcgagttctaccacaagtgtgacaacgagtgtatggagtctgtgagaaacggcacctacgattaccctcagtactctgaggaagccagactgaagcgcgaggagatctctggaaggctggtgccaagaggatctcctggcagcggatatattcctgaggcccctagagatggacaggcctatgtgagaaaggatggcgaatgggtgctgctgtctacatttctgggacaccaccaccatcaccattga

SEQ ID NO: 29 - DNA, 1707 bp, Influenza A
atggaaaagatcgtgctgctgctggccattgtgagcctggtgaagagcgaccagatctgcattggctaccacgccaacaatagcacagagcaggtggacaccatcatggaaaaaaacgtgaccgtgacccacgctcaggacatcctggaaaagacccacaacggcaagctgtgtgatctggacggcgtgaagcctctgatcctgagagattgtagcgtggctggatggctgctgggcaaccctatgtgcgacgagttcatcaacgtgcccgagtggagctatatcgtggagaaggccaaccccaccaacgatctgtgttaccccggcagcttcaacgattacgaggaactgaagcacctgctgtcccggatcaaccacttcgagaagatccagatcatccccaagtcctcttggagcgatcacgaagcctctagcggagtgtctagcgcctgtccttacctgggcagccccagcttcttcagaaacgtggtgtggctgatcaagaagaacagcacctaccccaccatcaagaagagctacaacaacaccaaccaggaagatctgctggtcctgtggggaatccaccaccctaatgatgccgccgatcagaccagcctgtaccagaaccccaccacctatatcagcatcggcaccagcaccctgaatcagagactggtgcccaagatcgccaccagatccaaggtgaacgatcagagcggcaggatggaattcttctggaccatcctgaagcccaacgacgccatcaacttcgagagcaacggcaactttatcgcccctgagtacgcctacaagatcgtgaagaagggcgacagcgccatcatgaagagcgagctggaatacggcaactgcaacaccaagtgccagacacctatgggcgccatcaacagcagcatgcccttccacaacatccaccctctgaccatcggcgagtgccctaagtacgtgaagagcaacagactggtgctggccacaggcctgagaaatagcccccagcgggagagcagaagaaagaagaggggcctgtttggagccatcgccggctttattgaaggcggctggcagggaatggtggatggctggtacggctaccaccacagcaatgagcagggctctggatatgccgccgacaaagagtctacccagaaggccatcgacggcgtcaccaacaaggtgaacagcatcatcgacaagatgaacacccagttcgaggctgtgggcagagagttcaacaacctggaacggcggatcgagaacctgaacaagaaaatggaagatggcttcctggatgtgtggacctacaatgccgaactgctggtgctgatggaaaacgagcggaccctggacttccacgacagcaacgtgaagaacctgtacgacaaagtgcggctgcagctgagagacaacgccaaagagctgggcaacggctgcttcgagttctaccacaagtgcgacaacgagtgcatggaaagcatccggaacggcacctacaactaccctcagtacagcgaggaagccaggctgaagagggaagagatcagcggcgtgaaactggaatccatcggcacctaccagatcctgagcatctacagcacagtggcctcttctctggccctggccattatgatggccggactgagcctgtggatgtgcagcaatggcagcctgcagtgcaggatctgcatctga

SEQ ID NO: 30 - DNA, 1695 bp, Influenza A
atggaaaagatcgtgctgctgctggccattgtgagcctggtgaagagcgaccagatctgcattggctaccacgccaacaatagcacagagcaggtggacaccatcatggaaaaaaacgtgaccgtgacccacgctcaggacatcctggaaaagacccacaacggcaagctgtgtgatctggacggcgtgaagcctctgatcctgagagattgtagcgtggctggatggctgctgggcaaccctatgtgcgacgagttcatcaacgtgcccgagtggagctatatcgtggagaaggccaaccccaccaacgatctgtgttaccccggcagcttcaacgattacgaggaactgaagcacctgctgtcccggatcaaccacttcgagaagatccagatcatccccaagtcctcttggagcgatcacgaagcctctagcggagtgtctagcgcctgtccttacctgggcagccccagcttcttcagaaacgtggtgtggctgatcaagaagaacagcacctaccccaccatcaagaagagctacaacaacaccaaccaggaagatctgctggtcctgtggggaatccaccaccctaatgatgccgccgatcagaccagcctgtaccagaaccccaccacctatatcagcatcggcaccagcaccctgaatcagagactggtgcccaagatcgccaccagatccaaggtgaacgatcagagcggcaggatggaattcttctggaccatcctgaagcccaacgacgccatcaacttcgagagcaacggcaactttatcgcccctgagtacgcctacaagatcgtgaagaagggcgacagcgccatcatgaagagcgagctggaatacggcaactgcaacaccaagtgccagacacctatgggcgccatcaacagcagcatgcccttccacaacatccaccctctgaccatcggcgagtgccctaagtacgtgaagagcaacagactggtgctggccacaggcctgagaaatagcccccagagagagaccagaggactgtttggagccatcgccggctttattgaaggcggctggcagggaatggtggatggctggtacggctaccaccacagcaatgagcagggctctggatatgccgccgacaaagagtctacccagaaggccatcgacggcgtcaccaacaaggtgaacagcatcatcgacaagatgaacacccagttcgaggctgtgggcagagagttcaacaacctggaacggcggatcgagaacctgaacaagaaaatggaagatggcttcctggatgtgtggacctacaatgccgaactgctggtgctgatggaaaacgagcggaccctggacttccacgacagcaacgtgaagaacctgtacgacaaagtgcggctgcagctgagagacaacgccaaagagctgggcaacggctgcttcgagttctaccacaagtgcgacaacgagtgcatggaaagcatccggaacggcacctacaactaccctcagtacagcgaggaagccaggctgaagagggaagagatcagcggcgtgaaactggaatccatcggcacctaccagatcctgagcatctacagcacagtggcctcttctctggccctggccattatgatggccggactgagcctgtggatgtgcagcaatggcagcctgcagtgcaggatctgcatctga

SEQ ID NO: 31 - DNA, 1686 bp, Influenza A
atggaaaagatcgtgctgctgctggccattgtgagcctggtgaagagcgaccagatctgcattggctaccacgccaacaatagcacagagcaggtggacaccatcatggaaaaaaacgtgaccgtgacccacgctcaggacatcctggaaaagacccacaacggcaagctgtgtgatctggacggcgtgaagcctctgatcctgagagattgtagcgtggctggatggctgctgggcaaccctatgtgcgacgagttcatcaacgtgcccgagtggagctatatcgtggagaaggccaaccccaccaacgatctgtgttaccccggcagcttcaacgattacgaggaactgaagcacctgctgtcccggatcaaccacttcgagaagatccagatcatccccaagtcctcttggagcgatcacgaagcctctagcggagtgtctagcgcctgtccttacctgggcagccccagcttcttcagaaacgtggtgtggctgatcaagaagaacagcacctaccccaccatcaagaagagctacaacaacaccaaccaggaagatctgctggtcctgtggggaatccaccaccctaatgatgccgccgatcagaccagcctgtaccagaaccccaccacctatatcagcatcggcaccagcaccctgaatcagagactggtgcccaagatcgccaccagatccaaggtgaacgatcagagcggcaggatggaattcttctggaccatcctgaagcccaacgacgccatcaacttcgagagcaacggcaactttatcgcccctgagtacgcctacaagatcgtgaagaagggcgacagcgccatcatgaagagcgagctggaatacggcaactgcaacaccaagtgccagacacctatgggcgccatcaacagcagcatgcccttccacaacatccaccctctgaccatcggcgagtgccctaagtacgtgaagagcaacagactggtgctggccacaggcctgagaaatagcccccagagagagaccagaggactgtttggagccatcgccggctttattgaaggcggctggcagggaatggtggatggctggtacggctaccaccacagcaatgagcagggctctggatatgccgccgacaaagagtctacccagaaggccatcgacggcgtcaccaacaaggtgaacagcatcatcgacaagatgaacacccagttcgaggctgtgggcagagagttcaacaacctggaacggcggatcgagaacctgaacaagaaaatggaagatggcttcctggatgtgtggacctacaatgccgaactgctggtgctgatggaaaacgagcggaccctggacttccacgacagcaacgtgaagaacctgtacgacaaagtgcggctgcagctgagagacaacgccaaagagctgggcaacggctgcttcgagttctaccacaagtgcgacaacgagtgcatggaaagcatccggaacggcacctacaactaccctcagtacagcgaggaagccaggctgaagagggaagagatcagcggcaggctggtgccaagaggatctcctggcagcggatatattcctgaggcccctagagatggacaggcctatgtgagaaaggatggcgaatgggtgctgctgtctacatttctgggacaccaccaccatcaccattga

SEQ ID NO: 32 - DNA, 1707 bp, Influenza A
atggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcgccgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagacaaagctgtaccagaatcctacaacatacatcagcgtgggaacaagcacactgaatcagagactggtgcctagaatcgccacaagaagcaaggtgaatggacagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagagaagaagaaagaagagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctga

SEQ ID NO: 33 - DNA, 1695 bp, Influenza A
atggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcgccgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagacaaagctgtaccagaatcctacaacatacatcagcgtgggaacaagcacactgaatcagagactggtgcctagaatcgccacaagaagcaaggtgaatggacagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagacgagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctga

SEQ ID NO: 34 - DNA, 1686 bp, Influenza A
atggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcgccgcctgcccttaccagagaaagagcagcttcttcagaaatgtggtgtggctgatcaagaagaacagcacctaccccacaatcaagcggagctacaacaacaccaaccaggaagatctgctggtcctgtggggaattcaccatcctaatgatgccgccgagcagacaaagctgtaccagaaccccaccacatatatctctgtgggcaccagcacactgaatcagagactggtgcctagaatcgccacaagatccaaggtgaacggccagtctggcagaatggagttcttctggaccatcctgaagccaaacgacgccatcaacttcgagagcaacggcaatttcatcgcccctgagtacgcctataagatcgtgaagaagggcgatagcaccatcatgaagagcgagctggagtacggcaactgtaataccaagtgccagacacctatgggcgccatcaatagctctatgcccttccacaatatccaccctctgacaatcggcgagtgtcctaagtacgtgaagagcaacagactggtgctggctacaggcctgagaaatagccctcagagagagacaagaggactgtttggagccatcgccggattcattgaagggggatggcagggaatggtcgatggctggtatggctatcaccacagcaatgagcagggatctggatatgccgccgataaggagtctacacagaaggccatcgacggcgtcacaaacaaggtgaacagcatcatcgacaagatgaacacccagtttgaggctgtgggcagagagttcaacaacctggagcggagaatcgagaacctgaacaagaagatggaggacggctttctggatgtgtggacctataatgccgaactgctggtgctgatggagaacgagagaaccctggatttccacgacagcaacgtgaagaacctgtacgacaaagtgagactgcagctgagagataatgccaaggaactgggcaatggctgcttcgagttctaccacaagtgtgacaacgagtgtatggagtctgtgagaaacggcacctacgattaccctcagtactctgaggaagccagactgaagcgcgaggagatctctggaaggctggtgccaagaggatctcctggcagcggatatattcctgaggcccctagagatggacaggcctatgtgagaaaggatggcgaatgggtgctgctgtctacatttctgggacaccaccaccatcaccattga

SEQ ID NO: 35 - DNA, 1707 bp, Influenza A
atggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcagcgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagatcaagctgtaccagaatcctacaacatacatcagcgtgggaacaagcacactgaatcagagactggtgcctagaatcgccacaagaagcaaggtgaatggacagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagagaagaagaaagaagagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctga

SEQ ID NO: 36 - DNA, 1695 bp, Influenza A
atggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcagcgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagatcaagctgtaccagaatcctacaacatacatcagcgtgggaacaagcacactgaatcagagactggtgcctagaatcgccacaagaagcaaggtgaatggacagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagacgagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctga

SEQ ID NO: 37 - DNA, 1686 bp, Influenza A
atggagaagattgtgctgctgttcgccattgtgagcctggtgaagagcgatcagatctgtattggctaccacgccaacaattctacagagcaggtggacaccatcatggagaaaaacgtgacagtgacacacgctcaggacatcctggagaaaacccacaatggcaagctgtgtgatctggatggagtgaagcctctgatcctgagagattgttctgtggctggatggctgctgggaaaccctatgtgtgacgagttcatcaatgtgcctgagtggagctatatcgtggagaaggccaaccctgtgaatgatctgtgttaccccggcgacttcaatgattacgaggagctgaagcacctgctgtccagaatcaaccacttcgagaagatccagatcatccctaagtctagctggtctagccatgaagcttctctgggagtgtctagcgcttgtccctatcagagaaagagcagcttcttcagaaatgtggtgtggctgatcaagaagaacagcacctaccccacaatcaagcggagctacaacaacaccaaccaggaagatctgctggtcctgtggggaattcaccatcctaatgatgccgccgagcagatcaagctgtaccagaaccccaccacatatatctctgtgggcaccagcacactgaatcagagactggtgcctagaatcgccacaagatccaaggtgaacggccagtctggcagaatggagttcttctggaccatcctgaagccaaacgacgccatcaacttcgagagcaacggcaatttcatcgcccctgagtacgcctataagatcgtgaagaagggcgatagcaccatcatgaagagcgagctggagtacggcaactgtaataccaagtgccagacacctatgggcgccatcaatagctctatgcccttccacaatatccaccctctgacaatcggcgagtgtcctaagtacgtgaagagcaacagactggtgctggctacaggcctgagaaatagccctcagagagagacaagaggactgtttggagccatcgccggattcattgaagggggatggcagggaatggtcgatggctggtatggctatcaccacagcaatgagcagggatctggatatgccgccgataaggagtctacacagaaggccatcgacggcgtcacaaacaaggtgaacagcatcatcgacaagatgaacacccagtttgaggctgtgggcagagagttcaacaacctggagcggagaatcgagaacctgaacaagaagatggaggacggctttctggatgtgtggacctataatgccgaactgctggtgctgatggagaacgagagaaccctggatttccacgacagcaacgtgaagaacctgtacgacaaagtgagactgcagctgagagataatgccaaggaactgggcaatggctgcttcgagttctaccacaagtgtgacaacgagtgtatggagtctgtgagaaacggcacctacgattaccctcagtactctgaggaagccagactgaagcgcgaggagatctctggaaggctggtgccaagaggatctcctggcagcggatatattcctgaggcccctagagatggacaggcctatgtgagaaaggatggcgaatgggtgctgctgtctacatttctgggacaccaccaccatcaccattga

SEQ ID NO: 38 - DNA, 1707 bp, Influenza A
atggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcgccgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagatcaagctgtaccagaatcctacaacatacatcagcgtgggaacaagcacactgaatcagagactggtgcctagaatcgccacaagaagcaaggtgaatggacagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagagaagaagaaagaagagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctga

SEQ ID NO: 39 - DNA, 1695 bp, Influenza A
atggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcgccgcctgcccttaccagagaaagagcagcttcttcagaaatgtggtgtggctgatcaagaagaatagcacataccctacaatcaagagaagctacaataatacaaatcaggaggatctgctggtgctgtggggaatccaccaccctaatgatgccgccgagcagatcaagctgtaccagaatcctacaacatacatcagcgtgggaacaagcacactgaatcagagactggtgcctagaatcgccacaagaagcaaggtgaatggacagagcggaagaatggagttcttctggacaatcctgaagcctaatgatgccatcaatttcgagagcaatggaaatttcatcgctcctgagtacgcctacaagatcgtgaagaagggagatagcacaatcatgaagagcgagctggagtacggaaattgcaatacaaagtgccagacacctatgggagccatcaatagcagcatgcctttccacaatatccaccctctgacaatcggagagtgccctaagtacgtgaagagcaatagactggtgctggccacaggactgagaaatagccctcagagagagacgagaggactgttcggagccatcgccggattcatcgagggaggatggcagggaatggtggatggatggtacggataccaccacagcaatgagcagggaagcggatacgccgccgataaggagagcacacagaaggccatcgatggagtgacaaataaggtgaatagcatcatcgataagatgaatacacagttcgaggccgtgggaagagagttcaataatctggagagaagaatcgagaatctgaataagaagatggaggatggattcctggatgtgtggacatacaatgccgagctgctggtgctgatggagaatgagagaacactggatttccacgatagcaatgtgaagaatctgtacgataaggtgagactgcagctgagagataatgccaaggagctgggaaatggatgcttcgagttctaccacaagtgcgataatgagtgcatggagagcgtgagaaatggaacatacgattaccctcagtacagcgaggaggccagactgaagagagaggagatcagcggagtgaagctggagagcatcggaatctaccagatcctgagcatctacagcacagtggccagcagcctggccctggccatcatggtggccggactgagcctgtggatgtgcagcaatggaagcctgcagtgcagaatctgcatctga

SEQ ID NO: 40 - DNA, 1686 bp, Influenza A
atggagaagatcgtgctgctgttcgccatcgtgagcctggtgaagagcgatcagatctgcatcggataccacgccaataatagcacagagcaggtggatacaatcatggagaagaatgtgacagtgacacacgcccaggatatcctggagaagacacacaatggaaagctgtgcgatctggatggagtgaagcctctgatcctgagagattgcagcgtggccggatggctgctgggaaatcctatgtgcgatgagttcatcaatgtgcctgagtggagctacatcgtggagaaggccaatcctgtgaatgatctgtgctaccctggagatttcaatgattacgaggagctgaagcacctgctgagcagaatcaatcacttcgagaagatccagatcatccctaagagcagctggagcagccacgaggccagcctgggagtgagcgccgcctgcccttaccagagaaagagcagcttcttcagaaatgtggtgtggctgatcaagaagaacagcacctaccccacaatcaagcggagctacaacaacaccaaccaggaagatctgctggtcctgtggggaattcaccatcctaatgatgccgccgagcagatcaagctgtaccagaaccccaccacatatatctctgtgggcaccagcacactgaatcagagactggtgcctagaatcgccacaagatccaaggtgaacggccagtctggcagaatggagttcttctggaccatcctgaagccaaacgacgccatcaacttcgagagcaacggcaatttcatcgcccctgagtacgcctataagatcgtgaagaagggcgatagcaccatcatgaagagcgagctggagtacggcaactgtaataccaagtgccagacacctatgggcgccatcaatagctctatgcccttccacaatatccaccctctgacaatcggcgagtgtcctaagtacgtgaagagcaacagactggtgctggctacaggcctgagaaatagccctcagagagagacaagaggactgtttggagccatcgccggattcattgaagggggatggcagggaatggtcgatggctggtatggctatcaccacagcaatgagcagggatctggatatgccgccgataaggagtctacacagaaggccatcgacggcgtcacaaacaaggtgaacagcatcatcgacaagatgaacacccagtttgaggctgtgggcagagagttcaacaacctggagcggagaatcgagaacctgaacaagaagatggaggacggctttctggatgtgtggacctataatgccgaactgctggtgctgatggagaacgagagaaccctggatttccacgacagcaacgtgaagaacctgtacgacaaagtgagactgcagctgagagataatgccaaggaactgggcaatggctgcttcgagttctaccacaagtgtgacaacgagtgtatggagtctgtgagaaacggcacctacgattaccctcagtactctgaggaagccagactgaagcgcgaggagatctctggaaggctggtgccaagaggatctcctggcagcggatatattcctgaggcccctagagatggacaggcctatgtgagaaaggatggcgaatgggtgctgctgtctacatttctgggacaccaccaccatcaccattga

SEQ ID NO: 41 - protein, 25 aa, Homo sapiens
VQLVQSGAEVKKPGASVKVSCKASG

SEQ ID NO: 42 - protein, 15 aa, Homo sapiens
WVRQAPGQGLEWMGW

SEQ ID NO: 43 - protein, 30 aa, Homo sapiens
TMTADTSISTAYMELSRLRSDDTAVYYCAR

SEQ ID NO: 44 - protein, 11 aa, Homo sapiens
WGQGTMVTVSS

SEQ ID NO: 45 - protein, 9 aa, Homo sapiens
YIFSEYIIN

SEQ ID NO: 46 - protein, 18 aa, Homo sapiens
FYPGSGSVKYNEKFNDKA

SEQ ID NO: 47 - protein, 9 aa, Homo sapiens
HERDGYYVY

SEQ ID NO: 48 - protein, 26 aa, Homo sapiens [sequence omitted]

SEQ ID NO: 49 - protein, 17 aa, Homo sapiens
MHWYQQKPGQAPRLLIY

SEQ ID NO: 50 - protein, 36 aa, Homo sapiens
NLETGIPARFSGSGSGTDFTLTIDPLEAEDVATYYC

SEQ ID NO: 51 - protein, 10 aa, Homo sapiens
FGQGTKVEIK

SEQ ID NO: 52 - protein, 10 aa, Homo sapiens
ESVDSFGNSF

SEQ ID NO: 53 - protein, 3 aa, Homo sapiens
LAS

SEQ ID NO: 54 - protein, 9 aa, Homo sapiens
QQNNEDPYT

SEQ ID NO: 55 - protein, 467 aa, Homo sapiens
MDWTWRILFLVAAATGAHSQVQLVQSGAEVKKPGASVKVSCKASGYIFSEYIINWVRQAPGQGLEWMGWFYPGSGSVKYNEKFNDKATMTADTSISTAYMELSRLRSDDTAVYYCARHERDGYYVYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 238 aa, Homo sapiens
MEAPAQLLFLLLLWLPDTTGEIVLTQSPATLSLSPGERATLSCRASESVDSFGNSFMHWYQQKPGQAPRLLIYLASNLETGIPARFSGSGSGTDFTLTIDPLEAEDVATYYCQQNNEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - DNA, 1404 bp, Homo sapiens
atggattggacatggagaatcctgttcctggtggctgctgctacaggagctcatagccaggtgcagctggtgcagagcggagctgaagtgaagaagcctggagctagcgtgaaggtgtcctgtaaggcctccggatacatcttcagcgagtacatcatcaactgggtgagacaggctcctggacagggactggaatggatgggatggttctaccctggaagcggaagcgtgaagtacaacgagaagttcaacgacaaggctacaatgacagctgacacaagcatctccacagcttacatggaactgtccagactgagaagcgatgatacagctgtgtactactgtgccagacacgaaagagacggatactacgtgtactggggacagggaacaatggtgaccgtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 58 - DNA, 717 bp, Homo sapiens
atggaagcccctgctcagctcctgtttctgctgctgctgtggctgcctgatacaacaggagaaatcgtgctgacacagagccctgccacactgagcctgagccctggagaaagagccacactgagctgcagagcctccgaaagcgtggattccttcggaaacagcttcatgcactggtaccagcagaagcctggacaggcccccagactgctgatctacctggcctccaacctggaaacaggaatccctgccagattttccggaagcggaagcggaacagatttcacactgacaatcgaccctctggaagctgaagatgtggctacatactactgtcagcagaacaacgaagatccttacacatttggacagggaacaaaggtggagatcaagagaacagtggccgccccttccgtgttcatcttccctccttccgacgaacagctgaaaagcggaacagccagcgtggtgtgtctgctgaacaacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctga

SEQ ID NO: 59 - protein, 137 aa, Mus musculus
MGWSWIFLFLLSVTAGVHSKVQLQQSGAELVKPGASVKLSCKASGYIFSEYIINWVKQKSGQGLEWIAWFYPGSGSVKYNEKFNDKATLSADTSSNTVYMELIRVTSEDSAVYFCARHERDGYYVYWGQGTTLTVSS

SEQ ID NO: 60 - protein, 131 aa, Mus musculus
METDTLLLWVLLLWVPGSTGNIVLTQSPASLAVSLGQRATISCRTSESVDSFGNSFMHWYQQKPGQPPKLLIYLASNLESGVPARFSGSGSRTDFTLTIDPVEADDVATYYCQQNNEDPYTFGGGTKLEIK

SEQ ID NO: 61 - DNA, 411 bp, Mus musculus
atgggatggagctggatctttctcttcctcctgtcagtaactgcaggtgtccactccaaggtccagctgcaacagtctggagctgagctggtgaaacccggggcttcagtgaagctgtcctgcaaggcttctggctacatcttcagtgaatatattataaattgggtcaagcagaaatctggacagggtcttgagtggattgcgtggttttaccctggaagtggtagtgtaaagtacaatgagaaattcaacgacaaggccacattgagtgcggacacgtcctccaacacagtctatatggagcttattagagtgacatctgaagactctgcggtctatttctgtgcaagacacgaaagggatggttactacgtctactggggccaaggcaccactctcacagtctcctca

SEQ ID NO: 62 - DNA, 393 bp, Mus musculus
atggagacagacacactcctgctatgggtgctgctgctctgggttccaggttccacaggtaacattgtgctgacccaatctccagcttctttggctgtgtctctaggacagagggccaccatatcctgcagaaccagtgaaagtgttgatagttttggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgttgcaacctattactgtcagcaaaataatgaagatccgtacacgttcggaggggggaccaagctggaaataaaa

SEQ ID NO: 63 - protein, 25 aa, Mus musculus
VQLQQSGAVLMKPGASVKISCKATG

SEQ ID NO: 64 - protein, 14 aa, Mus musculus
WVKQRPGHGLEWIG

SEQ ID NO: 65 - protein, 30 aa, Mus musculus
AFTADTSSNTANIQLTSLTSEDSAVYYCAR

SEQ ID NO: 66 - protein, 11 aa, Mus musculus
WGAGTTVTVSS

SEQ ID NO: 67 - protein, 9 aa, Mus musculus
YTFSSYWIE

SEQ ID NO: 68 - protein, 19 aa, Mus musculus
EILPGSGSINYNEIFKDKA

SEQ ID NO: 69 - protein, 14 aa, Mus musculus
GGYGYDPLYWSFDV

SEQ ID NO: 70 - protein, 26 aa, Mus musculus
DILLTQSPAILSVSPGERVSFSCRAS

SEQ ID NO: 71 - protein, 17 aa, Mus musculus
IHWYQQRTNGSPRLLIQ

SEQ ID NO: 72 - protein, 36 aa, Mus musculus
ESISGIPSRFSGSGSGTNFTLTINSVESEDIADYYC

SEQ ID NO: 73 - protein, 10 aa, Mus musculus
FGGGTKLEIK

SEQ ID NO: 74 - protein, 6 aa, Mus musculus
QSIGTN

SEQ ID NO: 75 - protein, 3 aa, Mus musculus
SAS

SEQ ID NO: 76 - protein, 9 aa, Mus musculus
QLTNTWPMT

SEQ ID NO: 77 - protein, 466 aa, Mus Musculus
MGWSWIFLFLLSVTAGVHSQVQLQQSGAVLMKPGASVKISCKATGYTFSSYWIEWVKQRPGHGLEWIGEILPGSGSINYNEIFKDKAAFTADTSSNTANIQLTSLTSEDSAVYYCARGGYGYDPLYWSFDVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 78 - protein, 234 aa, Mus musculus
MESQSQVFVFLLFWIPASRGDILLTQSPAILSVSPGERVSFSCRASQSIGTNIHWYQQRTNGSPRLLIQSASESISGIPSRFSGSGSGTNFTLTINSVESEDIADYYCQLTNTWPMTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 79 - DNA, 1404 bp, Mus musculus
atgggatggagctggatctttctcttcctcctgtcagtaactgctggtgtccactcccaggttcagctgcagcaatctggagctgtactgatgaagcctggggcctcagtgaagatttcctgcaaggctactggctacacattcagtagctactggatagagtgggtgaagcagaggcctggacatggccttgagtggattggagagattttacctggaagtggtagtattaattacaatgagatcttcaaggacaaggccgcattcactgcagatacatcctccaacacagccaacatacaactcaccagcctgacatctgaggactctgccgtctattactgtgcaaggggaggctatggttacgacccactctactggtccttcgatgtctggggcgcagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggttccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatga

SEQ ID NO: 80 - DNA, 708 bp, Mus musculus
atggagtcacagtctcaggtctttgtatttttgcttttctggattccagcctccagaggtgacatcttgctgactcagtctccagccatcctgtctgtgagtccaggagaaagagtcagtttctcctgcagggccagtcagagcattggcacaaacatacactggtatcagcaaagaacaaatggttctccaaggcttctcatacagtctgcttctgagtctatttctgggatcccgtccaggtttagtggcagtggatcagggacaaattttactctaaccatcaacagtgtggagtctgaagatattgcagattattactgtcaacttactaatacctggccaatgacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttgatga

SEQ ID NO: 81 - DNA, 1704 bp, Influenza A
atggagaaaatagtgcttcttcttgcaatagtcagtcttgttaaaagtgatcagatttgcattggttaccatgcaaacaattcaacagagcaggttgacacaatcatggaaaagaacgttactgttacacatgcccaagacatactggaaaagacacacaacgggaagctctgcgatctagatggagtgaagcctctaattttaagagattgtagtgtagctggatggctcctcgggaacccaatgtgtgacgaattcatcaatgtaccggaatggtcttacatagtggagaaggccaatccaaccaatgacctctgttacccagggagtttcaacgactatgaagaactgaaacacctattgagcagaataaaccattttgagaaaattcaaatcatccccaaaagttcttggtccgatcatgaagcctcatcaggagtgagctcagcatgtccatacctgggaagtccctccttttttagaaatgtggtatggcttatcaaaaagaacagtacatacccaacaataaagaaaagctacaataataccaaccaagaagatcttttggtactgtggggaattcaccatcctaatgatgcggcagagcagacaaggctatatcaaaacccaaccacctatatttccattgggacatcaacactaaaccagagattggtaccaaaaatagctactagatccaaagtaaacgggcaaagtggaaggatggagttcttctggacaattttaaaacctaatgatgcaatcaacttcgagagtaatggaaatttcattgctccagaatatgcatacaaaattgtcaagaaaggggactcagcaattatgaaaagtgaattggaatatggtaactgcaacaccaagtgtcaaactccaatgggggcgataaactctagtatgccattccacaacatacaccctctcaccatcggggaatgccccaaatatgtgaaatcaaacagattagtccttgcaacagggctcagaaatagccctcaaagagagagcagaagaaaaaagagaggactatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcaatcattgacaaaatgaacactcagtttgaggccgttggaagggaatttaataacttagaaaggagaatagagaatttaaacaagaagatggaagacgggtttctagatgtctggacttataatgccgaacttctggttctcatggaaaatgagagaactctagactttcatgactcaaatgttaagaacctctacgacaaggtccgactacagcttagggataatgcaaaggagctgggtaacggttgtttcgagttctatcacaaatgtgataatgaatgtatggaaagtataagaaacggaacgtacaactatccgcagtattcagaagaagcaagattaaaaagagaggaaataagtggggtaaaattggaatcaataggaacttaccaaatactgtcaatttattcaacagtggcgagttccctagcactggcaatcatgatggctggtctatctttatggatgtgctccaatggatcgttacaatgcagaatttgcatt

SEQ ID NO: 82 - protein, 568 aa, Influenza A
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRESRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLESIGTYQILSIYSTVASSLALAIMMAGLSLWMCSNGSLQCRICI

SEQ ID NO: 83 - DNA, 1701 bp, Influenza A
atggagaaaatagtgcttcttcttgcaatagtcagccttgttaaaagtgatcagatttgcattggttaccatgcaaacaactcgacagagcaggttgacacaataatggaaaagaacgttactgttacacatgcccaagacatactggaaaagacacacaacgggaagctctgcgatctagatggagtgaagcctctgattttaagagattgtagtgtagctggatggctcctcggaaacccaatgtgtgacgaattcatcaatgtgccggaatggtcttacatagtggagaaggccaacccagccaatgacctctgttacccagggaatttcaacgactatgaagaactgaaacacctattgagcagaataaaccattttgagaaaattcagatcatccccaaaagttcttggtccgatcatgaagcctcatcaggggtgagctcagcatgtccataccagggaacgccctcctttttcagaaatgtggtatggcttatcaaaaagaacaatacatacccaacaataaagagaagctacaataataccaaccaggaagatcttttgatactgtgggggattcatcattctaatgatgcggcagagcagacaaagctctatcaaaacccaaccacctatatttccgttgggacatcaacactaaaccagagattggtaccaaaaatagctactagatccaaagtaaacgggcaaagtggaaggatggatttcttctggacaattttaaaaccgaatgatgcaatcaacttcgagagtaatggaaatttcattgctccagaatatgcatacaaaattgtcaagaaaggggactcagcaattgttaaaagtgaagtggaatatggtaactgcaacacaaagtgtcaaactccaataggggcgataaactctagtatgccattccacaacatacaccctctcaccatcggggaatgccccaaatatgtgaaatcaaacaaattagtccttgcgactgggctcagaaatagtcctctaagagaaagaagaagaaaaagaggactatttggagctatagcagggtttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaataacttagaaaggagaatagagaatttaaacaagaaaatggaagacggattcctagatgtctggacttataatgctgaacttctggttctcatggaaaatgagagaactctagacttccatgattcaaatgtcaagaacctttacgacaaggtccgactacagcttagggataatgcaaaggagctgggtaacggttgtttcgagttctatcacaaatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagttgcgagttctctagcactggcaatcatggtggctggtctatctttgtggatgtgctccaatgggtcgttacaatgcagaatttgcatt

SEQ ID NO: 84 - protein, 567 aa, Influenza A
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGTPSFFRNVVWLIKKNNTYPTIKRSYNNTNQEDLLILWGIHHSNDAAEQTKLYQNPTTYISVGTSTLNQRLVPKIATRSKVNGQSGRMDFFWTILKPNDAINFESNGNFIAPEYAYKIVKKGDSAIVKSEVEYGNCNTKCQTPIGAINSSMPFHNIHPLTIGECPKYVKSNKLVLATGLRNSPLRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 85 - protein, 47 aa, Influenza A
VTQNGGSNACKRGPSTNQEQTSLYVQASGRIGSRPWVRGLSSRISIY

(19) United States
(12) Patent Application Publication
(10) Pub. No.: US 2010/0074916 A1
(43) Pub. Date: Mar. 25, 2010

(54) AVIAN INFLUENZA VACCINE

(75) Inventors: Gary J. Nabel, Washington, DC (US); Zhi-yong Yang, Potomac, MD (US); Chih-jen Wei, Gaithersburg, MD (US); Wing-pui Kong, Germantown, MD (US); Lan Wu, Washington, DC (US)

Correspondence Address: NIH-OTT, 1560 Broadway, Suite 1200, Denver, CO, 80238, US

(73) Assignee: Government of the United States of America, as represented by the Secretary, Dept. of Health and, Rockville, MD (US)

(21) Appl. No.: 12/443,964

(22) PCT Filed: Oct. 10, 2007
(86) PCT No.: PCT/US07/81002

§ 371 Date: Apr. 1, 2009

Related U.S. Application Data
(60) Provisional application No. 60/850,761, filed on Oct. 10, 2006.
(60) Provisional application No. 60/860,301, filed on Nov. 20, 2006.
(60) Provisional application No. 60/920,874, filed on Mar. 30, 2007.
(60) Provisional application No. 60/921,669, filed on Apr. 2, 2007.

(51) Int. Cl. A61K 39/145; C07K 14/005; C07K 16/00; C07H 21/04; C07H 21/02; C12P 21/00

(52) U.S. Cl. 424/189.1; 530/350; 530/387.9; 536/23.72; 435/69.1

(57) ABSTRACT

H5 hemagglutinin (HA) polypeptides are provided that are adapted to humans through mutations that change receptor specificity in the H1 serotype, and related polynucleotides, methods, compositions, and vaccines.

### RELATED APPLICATIONS

This application claims the benefit of U.S. Provisional Application No. 60/850,761 filed Oct. 10, 2006, U.S. Provisional Application No. 60/860,301 filed Nov. 20, 2006, U.S. Provisional Application No. 60/920,874 filed Mar. 30, 2007, and U.S. Provisional Application No. 60/921,669 filed Apr. 2, 2007, all of which are hereby expressly incorporated by reference in their entireties.

### FIELD OF THE INVENTION

The invention relates to immunogenic compositions and methods of use as vaccines against avian influenza viruses.

### DESCRIPTION OF THE RELATED ART

The ability of influenza viruses to adapt from animals to humans is determined by several viral gene products (reviewed in Parrish, C. R. et al. 2005 Annu Rev Microbiol 59:553). Among them, the viral hemagglutinin (HA) is of particular interest; it binds to specific sialic acid (SA) receptors in the respiratory tract that affect transmission (Parrish, C. R. et al. 2005 Annu Rev Microbiol 59:553; Bean, W. J. et al. 1992 J Virol 66:1129; Vines, A. et al. 1998 J Virol 72:7626). At the same time, it affects sensitivity to neutralizing antibodies, the primary determinant of immune protection (Subbarao, K. et al. 2006 Immunity 24:5; B. R. Murphy and R. G. Webster, in Fields Virology, D. M. Knipe et al., Eds. (Lippincott, Philadelphia, ed. 3, 1996), p. 1403).

### SUMMARY OF THE INVENTION

H5 hemagglutinin (HA) polypeptides are provided that are adapted to humans through mutations that change receptor specificity in the H1 serotype, and related polynucleotides, methods, compositions, and vaccines.

An embodiment of the invention is related to an isolated or recombinant hemagglutinin (HA) polypeptide, which polypeptide is selected from the group consisting of:
(a) a polypeptide having the amino acid sequence of SEQ ID NO:2;
(b) a polypeptide having the amino acid sequence of SEQ ID NO: 82;
(c) a polypeptide having the amino acid sequence of SEQ ID NO: 84;
(d) a polypeptide encoded by a polynucleotide sequence which hybridizes under highly stringent conditions over substantially the entire length of a polynucleotide sequence encoding (a) (b), or (c);
(e) a polypeptide sequence comprising a fragment of (a), (b), or (c), the polypeptide comprising an amino acid sequence which is substantially identical over at least about 350 amino acids; over at least about 400 amino acids; over at least about 450 amino acids; or over at least about 500 amino acids contiguous of said (a), (b), or (c); and
(f) a H5 HA polypeptide;
wherein said polypeptide comprises a mutation 5137 to an amino acid other than S, that is, A, R, N, D, C, E, Q, G, H, I, L, K, M, F, P, T, W, Y, or V, preferably A, and, optionally, a further mutation T192 to an amino acid other than T, that is, A, R, N, D, C, E, Q, G, H, L, K, M, F, P, S, T, W, Y, or V, preferably I.

Another embodiment of the invention is related to an isolated or recombinant hemagglutinin (HA) polypeptide, which polypeptide is selected from the group consisting of:
(a) a polypeptide having the amino acid sequence of SEQ ID NO:2;
(b) a polypeptide having the amino acid sequence of SEQ ID NO: 82;
(c) a polypeptide having the amino acid sequence of SEQ ID NO: 84;
(d) a polypeptide encoded by a polynucleotide sequence which hybridizes under highly stringent conditions over substantially the entire length of a polynucleotide sequence encoding (a) (b), or (c);
(e) a polypeptide sequence comprising a fragment of (a), (b), or (c), the polypeptide comprising an amino acid sequence which is substantially identical over at least about 350 amino acids; over at least about 400 amino acids; over at least about 450 amino acids; or over at least about 500 amino acids contiguous of said (a), (b), or (c); and
(f) a H5 HA polypeptide;
wherein said polypeptide comprises a mutation K/R193 to an amino acid other than K or R, that is, A, N, D, C, E, Q, G, H, I, L, M, F, P, S, T, W, Y, or V, preferably S, A, T or N, and at least one mutation selected from the group consisting of S136 to an amino acid other than S, that is, A, R, N, D, C, E, Q, G, H, I, L, K, M, F, P, T, W, Y, or V, preferably T, E190 to an amino acid other than E, that is, A, R, N, D, C, Q, G, H, I, L, K, M, F, P, S, T, W, Y, or V, preferably D, N, or G, L194 to an amino acid other than L, that is, A, R, N, D, C, E, Q, G, H, I, K, M, F, P, S, T, W, Y, or V, preferably I or F, R216 to an amino acid other than R, that is, A, N, D, C, E, Q, G, H, I, L, K, M, F, P, S, T, W, Y, or V, preferably E, S221 to an amino acid other than S, that is, A, R, N, D, C, E, Q, G, H, I, L, K, M, F, P, T, W, Y, or V, preferably P, K222 to an amino acid other than K, that is, A, R, N, D, C, E, Q, G, H, I, L, M, F, P, S, T, W, Y, or V, preferably W, G225 to an amino acid other than G, that is, A, R, N, D, C, E, Q, H, I, L, K, M, F, P, S, T, W, Y, or V, preferably D or N, Q226 to an amino acid other than Q, that is, A, R, N, D, C, E, G, H, I, L, K, M, F, P, S, T, W, Y, or V, preferably R or L, S227 to an amino acid other than S, that is, A, R, N, D, C, E, Q, G, H, I, L, K, M, F, P, T, W, Y, or V, preferably A, H, P, E, or N, and G228 to an amino acid other than G, that is, A, R, N, D, C, E, Q, H, I, L, K, M, F, P, S, T, W, Y, or V, preferably S.

Other embodiments of the invention are related to polypeptides comprising a sequence having at least 95% sequence identity thereto, immunogenic fragments thereof, compositions thereof, immunogenic compositions thereof, modifications of the cleavage site, modifications of the carboxy terminus to a trimerization site in place of the transmembrane domain, polynucleotide sequences encoding therefor, vectors, methods of making, methods of using, antibodies specific therefor, and antibodies 9B11, 10D10, 9E8, and 11H12.

### BRIEF DESCRIPTION OF THE DRAWINGS

FIG. 1. A schematic diagram of the structure of the influenza A virus particle.

FIG. 2. Diagram of Influenza A hemagglutinin protein.

FIG. 3. Influenza A virus (A/Thailand/1(KAN-1)/2004(H5N1)) hemagglutinin (HA); GenBank Accession No. AY555150; wild type; polypeptide sequence is SEQ ID NO: 2; and polynucleotide sequence is SEQ ID NO: 1.

FIG. 4. Structural and genetic basis for hemagglutinin mutations. (A) The RBDs of alternative viral hemagglutinins are shown. (B) Comparison of amino acid sequences in the major 130 and 220 loops and the 190 helix.

FIG. 5. Functional activity of HA NA pseudotyped lentiviral vectors: equivalent expression of wild-type and mutant H5 hemagglutinins, reactivity of 293A cells with both α2,3 and α2,6 SA-specific lectins, and ability of pseudotyped viruses containing wild type or mutant HAs in addition to neuraminidase to mediate entry. (A) The expression of wild-type or the indicated mutant influenza H5N1 HAs is shown in transfected 293T cells using flow cytometry (Paulson, J. C. and Rogers, G. N. 1987 Methods Enzymol 138:162); preimmune control (gray) or anti-H5 (black). (B) 293A cells were incubated with biotinylated-labeled MAA or SNA, analyzed by flow cytometry as indicated. (C) The efficiency of entry mediated by H5 (KAN-1) and its derivatives was analyzed after preparation of lentiviral vectors pseudotyped with the indicated HA wild-type (WT) or mutant variants in addition to NA as described in Example 1, measured using the luciferase assay. Expression levels for the indicated mutants were: Control, 4.78×10³; WT, 3.16×10⁸; Q226L, G228S, 3.79×10⁸; E190D, 1.55×10⁷; K193S, 3.78×10⁸; G225D, 2.97×10⁸; E190D,K193S, 4.51×10⁸; E190D,G225D, 8.3×10⁶; K193S,G225D, 4.03×10⁸; E190D,K193S,G225D, 3.05×10⁸.

FIG. 6. Altered specificity of the triple-mutant H5 compared with wild-type KAN-1H5 coexpressed with NA Glycan microarray analysis of (A) wild-type or (B) triple-mutant HA purified after coexpression with NA was performed by a modification (Example 1) of a previous technique (Stevens, J. Et al. 2006 Nat Rev Microbiol 4:857) performed by Core H, Consortium for Functional Genomics, Emory University. Glycans with related linkages are grouped by number: selected glycoproteins (1-6), predominantly 2,3-sialosides (7-44), 2,6-sialosides (45-60), 2,8 ligands (61-67), or others (68-84), as previously shown (Table 8).

FIG. 7. Altered neutralization sensitivity of mutant H5N1 pseudovirus. (A) Binding to HA coexpressed with NA in transfected 293T cells was determined by flow cytometry with the indicated mAbs (black) or isotype control IgG (gray). (B) Neutralization sensitivities were assessed with the indicated mAbs. (C) Neutralization sensitivities of the indicated wild-type and mutant HAs to these mAbs (400 ng/ml) are shown. (D) Neutralization sensitivities of wild-type and S137A, T1921 mutant to mAb 9E8 and 11H12 are presented.

FIG. 8. H5 (Kan-1) (E190D/K193S/G225D). Protein sequence (SEQ ID NO: 8), DNA sequence (SEQ ID NO: 26).

FIG. 9. H5 (Kan-1) (mut.A) (E190D/K193S/G225D). Protein sequence (SEQ ID NO: 9), DNA sequence (SEQ ID NO: 27).

FIG. 10. H5 (Kan-1) (mut.A) (short)/Foldon (E190D/K193S/G225D). Protein sequence (SEQ ID NO: 10), DNA sequence (SEQ ID NO: 28).

FIG. 11. H5 Indonesia (E190D/K193S/G225D). Protein sequence (SEQ ID NO: 11), DNA sequence (SEQ ID NO: 29).

FIG. 12. H5 Indonesia (mut.A) (E190D/K193S/G225D). Protein sequence (SEQ ID NO: 12), DNA sequence (SEQ ID NO: 30).

FIG. 13. H5 (Indonesia) (mut.A) (short)/Foldon (E190D/K193S/G225D). Protein sequence (SEQ ID NO: 13), DNA sequence (SEQ ID NO: 31).

FIG. 14. VRC9151 (SEQ ID NO: 14).

FIG. 15. VRC9152 (SEQ ID NO: 15).

FIG. 16. VRC9153 (SEQ ID NO: 16).

FIG. 17. H5 (Kan-1) (S137A). Protein sequence (SEQ ID NO: 17), DNA sequence (SEQ ID NO: 32).

FIG. 18. H5 (Kan-1) (mut.A) (S137A). Protein sequence (SEQ ID NO: 18), DNA sequence (SEQ ID NO: 33).

FIG. 19. H5 (Kan-1) (mut.A) (short)/Foldon (S137A). Protein sequence (SEQ ID NO: 19), DNA sequence (SEQ ID NO: 34).

FIG. 20. H5 (Kan-1) (T1921). Protein sequence (SEQ ID NO: 20), DNA sequence (SEQ ID NO: 35).

FIG. 21. H5 (Kan-1) (mut.A) (T1921). Protein sequence (SEQ ID NO: 21), DNA sequence (SEQ ID NO: 36).

FIG. 22. H5 (Kan-1) (mut.A) (short)/Foldon (T1921). Protein sequence (SEQ ID NO: 22), DNA sequence (SEQ ID NO: 37).

FIG. 23. H5 (Kan-1) (S137A/T1921). Protein sequence (SEQ ID NO: 23), DNA sequence (SEQ ID NO: 38).

FIG. 24. H5 (Kan-1) (mut.A) (S137A/T1921). Protein sequence (SEQ ID NO: 24), DNA sequence (SEQ ID NO: 39).

FIG. 25. H5 (Kan-1) (mut.A) (short)/Foldon (S137A/T1921). Protein sequence (SEQ ID NO: 25), DNA sequence (SEQ ID NO: 40).

FIG. 26. Influenza A virus (A/Indonesia/5/05(H5N1)) hemagglutinin (HA); GenBank Accession No. ISDN125873; wild type; polypeptide sequence is SEQ ID NO: 82; and polynucleotide sequence is SEQ ID NO: 81.

FIG. 27. Influenza A virus (A/Anhui/1/2005(H5N1)) hemagglutinin (HA); GenBank Accession No. ABD28180; wild type; polypeptide sequence is SEQ ID NO: 84; and polynucleotide sequence is SEQ ID NO: 83.

The following biological material has been deposited in accordance with the terms of the Budapest Treaty with the American Type Culture Collection (ATCC), Manassas, Va., on the date indicated:

| Biological material | Designation No. | Date |
|---|---|---|
| 10D10 Mouse B Cell hybridoma | PTA-7916 | Oct. 10, 2006 |
| 9B11 Mouse B Cell hybridoma | PTA-8306 | Apr. 02, 2007 |

### Deposit of Biological Material: 10D10

10D10 Mouse B Cell hybridoma was deposited as ATCC Accession No. PTA-7916 on Oct. 10, 2006 with the American Type Culture Collection (ATCC), 10801 University Blvd., Manassas, Va. 20110-2209, USA. This deposit was made under the provisions of the Budapest Treaty on the International Recognition of the Deposit of Microorganisms for the Purposes of Patent Procedure and the Regulations there under (Budapest Treaty). This assures maintenance of a viable culture of the deposit for 30 years from date of deposit. The deposit will be made available by ATCC under the terms of the Budapest Treaty, and subject to an agreement between Applicant and ATCC which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC §122 and the Commissioner's rules pursuant thereto (including 37 CFR §1.14). Availability of the deposited biological material is not to be construed as a license to practice the invention in contravention of the rights granted under the authority of any government in accordance with its patent laws.

### Deposit of Biological Material: 9B11

9B11 Mouse B Cell hybridoma was deposited as ATCC Accession No. PTA-8306 on Apr. 2, 2007 with the American Type Culture Collection (ATCC), 10801 University Blvd., Manassas, Va. 20110-2209, USA. This deposit was made under the provisions of the Budapest Treaty on the International Recognition of the Deposit of Microorganisms for the Purposes of Patent Procedure and the Regulations there under (Budapest Treaty). This assures maintenance of a viable culture of the deposit for 30 years from date of deposit. The deposit will be made available by ATCC under the terms of the Budapest Treaty, and subject to an agreement between Applicant and ATCC which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC §122 and the Commissioner's rules pursuant thereto (including 37 CFR §1.14). Availability of the deposited biological material is not to be construed as a license to practice the invention in contravention of the rights granted under the authority of any government in accordance with its patent laws.

### 9E8 Antibody Sequence Containing CDR and FR Regions

### I. Humanized Sequences

### Protein Sequences

Humanized 9E8 Heavy chain V regions:

(SEQ ID NO: 41)

FR1: VQLVQSGAEVKKLPGASVKVSCKASG

(SEQ ID NO: 42)

FR2: WVRQAPGQGLEWMGW

(SEQ ID NO: 43)

FR3: TMTADTSISTAYMELSRLRSDDTAVYYCAR

(SEQ ID NO: 44)

FR4: WGQGTMVTVSS

(SEQ ID NO: 45)

CDR1: YIFSEYIIN

(SEQ ID NO: 46)

CDR2: FYPGSGSVKYNEKFNDKA

(SEQ ID NO: 47)

CDR3: HERDGYYVY
Humanized 9E8 Kappa chain V regions:

(SEQ ID NO: 48)

FR1: EIVLTQSPATLSLSPGERATLSCRAS

(SEQ ID NO: 49)

FR2: MHWYQQKPGQAPRLLIY

(SEQ ID NO: 50)

FR3: NLETGIPARFSGSGSGTDFTLTIDPLEAEDVATYYC

(SEQ ID NO: 51)

FR4: FGQGTKVEIK

(SEQ ID NO: 52)

CDR1: ESVDSFGNSF

(SEQ ID NO: 53)

CDR2: LAS

(SEQ ID NO: 54)

CDR3: QQNNEDPYT
Humanized 9E8 heavy chain

(SEQ ID NO: 55)

mdwtwrilflvaaatgahsqvqlvqsgaevkkpgasvkvsckasgyifse
yiinwvrqapgqglewmgwfypgsgsvkynekfhdkatmtdtsistayme
lsrlrsddtavyycarherdgyyvywgqgtmvtvssastkgpsvfplaps
skstsggtaalgclvkdyfpepvtvswnsgaltsgvhtfpavlqssglvs
lssvvtvpssslgtqtvicnvnhkpsntkvdkkvepksedkthtcppcpa
pellggpsvflfppkpkdtlmisrtpevtcvvvdvshedpevkfinwyvd
gvevhnaktkpreeqynstyrvvsvltvlhqdwlngkeykckvsnkalpa
piektiskakgqprepqvytlppsrdeltknqvsltclvkgfypsdiave
wrsngqpennykltppvldsdgsfflyskttvdksrwqqgnvfscsvmhe
alhnhytqkslslspgk
Humanized 9E8 light chain

(SEQ ID NO: 56)

meapaqllfllllwlpdttgeivltqspatlslspgeratlscrasesvd
sfgnsfmhwyqqkpgqaprlliylasnletgiparfsgsgsgtdftltid
pleaedvatyycqqnnedpytfgqgtkveikrtvaapsvfifppsdeqlk
sgtasvvcllnnfypreakvqwkvdnalqsgnsqesvteqdskdstysls
stltlskadyekhkvyacevthqglsspvtkslfingec

Humanized 9E8 heavy chain

(SEQ ID NO: 57)

atggattggacatggagaatcctgttcctggtggctgctgctacaggagc
tcatagccaggtgcagctggtgcagagcggagctgaagtgaagaagcctg
gagctagcgtgaaggtgtcctgtaaggcctccggatacatcttcagcgag
tacatcatcaactgggtgagacaggctcctggacagggactggaatggat
gggatggttctaccctggaagcggaagcgtgaagtacaacgagaagttca
acgacaaggctacaatgacagctgacacaagcatctccacagcttacatg
gaactgtccagactgagaagcgatgatacagctgtgtactactgtgccag
acacgaaagagacggatactacgtgtactggggacagggaacaatggtga
ccgtgtcctccgcctccaccaagggcccatcggtcttccccctggcaccc
tcctccaagagcacctctgggggcacagcggccctgggctgcctggtcaa
ggactacttccccgaaccggtgacggtgtcgtggaactcaggcgccctga
ccagcggcgtgcacaccttcccggctgtcctacagtcctcaggactctac
tccctcagcagcgtggtgaccgtgccctccagcagcttgggcacccagac
ctacatctgcaacgtgaatcacaagcccagcaacaccaaggtggacaaga
aagttgagcccaaatcttgtgacaaaactcacacatgcccaccgtgccca
gcacctgaactcctggggggaccgtcagtcttcctcttccccccaaaacc
caaggacaccctcatgatctcccggacccctgaggtcacatgcgtggtgg
tggacgtgagccacgaagaccctgaggtcaagttcaactggtacgtggac
ggcgtggaggtgcataatgccaagacaaagccgcgggaggagcagtacaa
cagcacgtaccgtgtggtcagcgtcctcaccgtcctgcaccaggactggc
tgaatggcaaggagtacaagtgcaaggtctccaacaaagccctcccagcc
cccatcgagaaaaccatctccaaagccaaagggcagccccgagaaccaca
ggtgtacaccctgcccccatcccgggatgagctgaccaagaaccaggtca
gcctgacctgcctggtcaaaggcttctatcccagcgacatcgccgtggag
tgggagagcaatgggcagccggagaacaactacaagaccacgcctcccgt
gctggactccgacggctccttcttcctctacagcaagctcaccgtggaca
agagcaggtggcagcaggggaacgtcttctcatgctccgtgatgcatgag
gctctgcacaaccactacacgcagaagagcctctccctgtctccgggtaa
atga
Humanized 9E8 light chain

(SEQ ID NO: 58)

Atggaagcccctgctcagctcctgtttctgctgctgctgtggctgcctga
tacaacaggagaaatcgtgctgacacagagccctgccacactgagcctga
gccctggagaaagagccacactgagctgcagagcctccgaaagcgtggat
tccttcggaaacagcttcatgcactggtaccagcagaagcctggacaggc
ccccagactgctgatctacctggcctccaacctggaaacaggaatccctg
ccagattttccggaagcggaagcggaacagatttcacactgacaatcgac
cctctggaagctgaagatgtggctacatactactgtcagcagaacaacga
agatccttacacatttggacagggaacaaaggtggagatcaagagaacag
tggccgccccttccgtgttcatcttccctccttccgacgaacagctgaaa
agcggaacagccagcgtggtgtgtctgctgaacaacttctaccccagaga
agccaaagtgcagtggaaggtggacaacgccctgcagagcggaaacagcc
aggaaagcgtgacagagcaggattccaaggattccacatacagcctgagc
agcacactgacactgtccaaggccgactacgagaagcacaaggtgtacgc
ctgcgaagtgacacaccagggactgtcctcccctgtgacaaagagcttca
acagaggagaatgctga

### DNA Sequences

### II. Mouse Sequences

Mouse anti-H5(Kan-1) monoclonal antibody, 9E8 VH
(SEQ ID NO: 59):
mgwswiflfllsvtagvhskvqlqqsgaelvkpgasvklsckasgyifse
yiinwvkqksgqglewiawfypgsgsvkynekfndkatlsadtssntvym
elirvtsedsavyfcarherdgyyvywgqgttltvss
Mouse anti-H5(Kan-1) monoclonal antibody, 9E8 VL
(SEQ ID NO: 60):
metdtlllwvlllwvpgstgnivltqspaslavslgqratiscrtsesvd
sfgnsfmhwyqqkpgqppklliylasnlesgvparfsgsgsrtdftltid
pveaddvatyycqqnnedpytfgggtkleik

Mouse anti-H5(Kan-1) monoclonal antibody, 9E8 VH
(SEQ II) NO: 61):
atgggatggagctggatctttctcttcctcctgtcagtaactgcaggtgt
ccactccaaggtccagctgcaacagtctggagctgagctggtgaaacccg
gggcttcagtgaagctgtcctgcaaggcttctggctacatcttcagtgaa
tatattataaattgggtcaagcagaaatctggacagggtcttgagtggat
tgcgtggttttaccctggaagtggtagtgtaaagtacaatgagaaattca
acgacaaggccacattgagtgcggacacgtcctccaacacagtctatatg
gagcttattagagtgacatctgaagactctgcggtctatttctgtgcaag
acacgaaagggatggttactacgtctactggggccaaggcaccactctca
cagtctcctca
Mouse anti-H5(Kan-1) monoclonal antibody, 9E8 VL
(SEQ ID NO: 62):
atggagacagacacactcctgctatgggtgctgctgctctgggttccagg
ttccacaggtaacattgtgctgacccaatctccagcttctttggctgtgt
ctctaggacagagggccaccatatcctgcagaaccagtgaaagtgttgat
agttttggcaatagttttatgcactggtaccagcagaaaccaggacagcc
acccaaactcctcatctatcttgcatccaacctagaatctggggtccctg
ccaggttcagtggcagtgggtctaggacagacttcaccctcaccattgat
cctgtggaggctgatgatgttgcaacctattactgtcagcaaaataatga
agatccgtacacgttcggaggggggaccaagctggaaataaaa

### Protein Sequences

### DNA Sequences

### 11H12 Antibody Sequence Containing CDR and FR Regions

### Protein Sequences

Mus 11H12 Heavy chain V regions:

(SEQ ID NO: 63)

FR1: VQLQQSGAVLMKPGASVKISCKATG

(SEQ ID NO: 64)

FR2: WVKQRPGHGLEWIG

(SEQ ID NO: 65)

FR3: AFTADTSSNTANIQLTSLTSEDSAVYYCAR

(SEQ ID NO: 66)

FR4: WGAGTTVTVSS

(SEQ ID NO: 67)

CDR1: YTFSSYWIE

(SEQ ID NO: 68)

CDR2: EILPGSGSINYNEIFKDKA

(SEQ ID NO: 69)

CDR3: GGYGYDPLYWSFDV
Mus 11H12 Kappa chain V regions:

(SEQ ID NO: 70)

FR1: DILLTQSPAILSVSPGERVSFSCRAS

(SEQ ID NO: 71)

FR2: IHWYQQRTNGSPRLLIQ

(SEQ ID NO: 72)

FR3: ESISGIPSRFSGSGSGTNFTLTINSVESEDIADYYC

(SEQ ID NO: 73)

FR4: FGGGTKLEIK

(SEQ ID NO: 74)

CDRI: QSIGTN

(SEQ ID NO: 75)

CDR2:SAS

(SEQ ID NO: 76)

CDR3: QLTNTWPMT
11H12 Heavy chain (SEQ ID NO: 77):
mgwswiflfllsvtagvhsqvqlqqsgavlmkpgasvkisckatgytfss
ywiewvkqrpghglewigeilpgsgsinyneifkdkaaftadtssntani
qltslsedsavyycarggygydplywsfdvwgagttvtvssakttppsvy
plapgsaaqtnsmvtlgclvkgyfpepvtvtwnsgslssgvhtfpavlqs
dlytlsssvtvpsstwpsetvtcnvahpasstkvdkkivprdcgckpcic
tvpevssvfifppkpkdvltitltpkvtcvvvdiskddpevqfswfvddv
evhtaqtqpreeqfnstfrsvselpimhqdwlngkefkcrvnsaafpapi
ektisktkgrpkapqvytipppkeqmakdkvsltcmitdffpeditvewq
wngqpaenykntqpimdtdgsyfvysklnvqksnweagntftcsvlhegl
hnhhtekslshspgk
11H12 Light chain (SEQ ID NO: 78):
mesqsqvfvfllfwwipasrgdilltqspailsvspgervsfscrasqsi
gtnihwyqqrtngsprlliqsasesisgipsrfsgsgsgtnftltinsve
sediadyycqltntwpmtfgggtkleikradaaptvsifppsseqltsgg
asvvcflnnfypkdinvkwkidgserqngvlnswtdqdskdstysmsstl
tltkdeyerhnsytceathktstspivksfnrnec

### DNA Sequences

11H12 Heavy chain (SEQ ID NO: 79):
atgggatggagctggatctttctcttcctcctgtcagtaactgctggtgt
ccactcccaggttcagctgcagcaatctggagctgtactgatgaagcctg
gggcctcagtgaagatttcctgcaaggctactggctacacattcagtagc
tactggatagagtgggtgaagcagaggcctggacatggccttgagtggat
tggagagattttacctggaagtggtagtattaattacaatgagatcttca
aggacaaggccgcattcactgcagatacatcctccaacacagccaacata
caactcaccagcctgacatctgaggactctgccgtctattactgtgcaag
gggaggctatggttacgacccactctactggtccttcgatgtctggggcg
cagggaccacggtcaccgtctcctcagccaaaacgacacccccatctgtc
tatccactggcccctggatctgctgcccaaactaactccatggtgaccct
gggatgcctggtcaagggctatttccctgagccagtgacagtgacctgga
actctggttccctgtccagcggtgtgcacaccttcccagctgtcctgcag
tctgacctctacactctgagcagctcagtgactgtcccctccagcacctg
gcccagcgagaccgtcacctgcaacgttgcccacccggccagcagcacca
aggtggacaagaaaattgtgcccagggattgtggttgtaagccttgcata
tgtacagtcccagaagtatcatctgtcttcatcttccccccaaagcccaa
ggatgtgctcaccattactctgactcctaaggtcacgtgtgttgtggtag
acatcagcaaggatgatcccgaggtccagttcagctggtttgtagatgat
gtggaggtgcacacagctcagacgcaaccccgggaggagcagttcaacag
cactttccgctcagtcagtgaacttcccatcatgcaccaggactggctca
atggcaaggagttcaaatgcagggtcaacagtgcagctttccctgccccc
atcgagaaaaccatctccaaaaccaaaggcagaccgaaggctccacaggt
gtacaccattccacctcccaaggagcagatggccaaggataaagtcagtc
tgacctgcatgataacagacttcttccctgaagacattactgtggagtgg
cagtggaatgggcagccagcggagaactacaagaacactcagcccatcat
ggacacagatggctcttacttcgtctacagcaagctcaatgtgcagaaga
gcaactgggaggcaggaaatactttcacctgctctgtgttacatgagggc
ctgcacaaccaccatactgagaagagcctctcccactctcctggtaaatg
atga
11H12 Light chain (SEQ ID NO: 80):
atggagtcacagtctcaggtctttgtatttttgcttttctggattccagc
ctccagaggtgacatcttgctgactcagtctccagccatcctgtctgtga
gtccaggagaaagagtcagtttctcctgcagggccagtcagagcattggc
acaaacatacactggtatcagcaaagaacaaatggttctccaaggcttct
catacagtctgcttctgagtctatttctgggatcccgtccaggtttagtg
gcagtggatcagggacaaattttactctaaccatcaacagtgtggagtct
gaagatattgcagattattactgtcaacttactaatacctggccaatgac
gttcggtggaggcaccaagctggaaatcaaacgggctgatgctgcaccaa
ctgtatccatcttcccaccatccagtgagcagttaacatctggaggtgcc
tcagtcgtgtgcttcttgaacaacttctaccccaaagacatcaatgtcaa
gtggaagattgatggcagtgaacgacaaaatggcgtcctgaacagttgga
ctgatcaggacagcaaagacagcacctacagcatgagcagcaccctcacg
ttgaccaaggacgagtatgaacgacataacagctatacctgtgaggccac
tcacaagacatcaacttcacccattgtcaagagcttcaacaggaatgagt
gttgatga

TABLE 1
Influenza A HA Sequences and Plasmid Constructs

| | | SEQ ID | |
|---|---|---|---|
| Sequence/Construct Name | Description | NO | FIG. |
| H5 (Kan-1) | protein | 8 | 8 |
| (E190D/K193S/G225D) | DNA | 26 | |
| H5 (Kan-1) (mut.A) | protein | 9 | 9 |
| (E190D/K193S/G225D) | DNA | 27 | |
| H5 (Kan-1) (mut.A) | protein | 10 | 10 |
| (short)/Foldon | DNA | 28 | |
| (E190D/K193S/G225D) | | | |
| H5 Indonesia | protein | 11 | 11 |
| (E190D/K193S/G225D) | DNA | 29 | |
| H5 Indonesia | protein | 12 | 12 |
| (mut.A)(E190D/K193S/G225D) | DNA | 30 | |
| H5 (Indonesia) (mut.A) | protein | 13 | 13 |
| (short)/Foldon | DNA | 31 | |
| (E190D/K193S/G225D) | | | |
| VRC9151 | CMV/R 8kb Influenza H5 | 14 | 14 |
| | (A/Thailand/1(KAN-1)/2004) HA | | |
| | ((E190D/K193S))/h | | |
| VRC9152 | CMV/R 8kb Influenza H5 | 15 | 15 |
| | (A/Thailand/1(KAN-1)/2004) HA | | |
| | ((K193S, Q226L))/h | | |
| VRC9253 | CMV/R 8kb Influenza H5 | 16 | 16 |
| | (A/Thailand/1(KAN-1)/2004) HA | | |
| | ((K193S, Q226L, G228S))/h | | |
| H5 (Kan-1) (S137A) | protein | 17 | 17 |
| | DNA | 32 | |
| H5 (Kan-1) (mut.A) (S137A) | protein | 18 | 18 |
| | DNA | 33 | |
| H5 (Kan-1) (mut.A) | protein | 19 | 19 |
| (short)/Foldon (S137A) | DNA | 34 | |
| H5 (Kan-1) (T192I) | protein | 20 | 20 |
| | DNA | 35 | |
| H5 (Kan-1) (mut.A) (T192I) | protein | 21 | 21 |
| | DNA | 36 | |
| H5 (Kan-1) (mut.A) | protein | 22 | 22 |
| (short)/Foldon (T192I) | DNA | 37 | |
| H5 (Kan-1) (S137A/T192I) | protein | 23 | 23 |
| | DNA | 38 | |
| H5 (Kan-1) (mut.A) (S137A/ | protein | 24 | 24 |
| T192I) | DNA | 39 | |
| H5 (Kan-1) (mut.A) | protein | 25 | 25 |
| (short)/Foldon (S137A/T192I) | DNA | 40 | |

### DETAILED DESCRIPTION OF THE PREFERRED EMBODIMENT

Influenza virus entry is mediated by the receptor binding domain (RBD) of its spike, the hemagglutinin (HA). Adaptation of avian viruses to humans is associated with HA specificity for α2,6-rather than α2,3-linked sialic acid (SA) receptors. Here, we define mutations in influenza A subtype H5N1 (avian) HA that alter its specificity for SA either by decreasing α2,3- or increasing α2,6-SA recognition. RBD mutants were used to develop vaccines and monoclonal antibodies that neutralized new variants. Structure-based modification of HA specificity can guide the development of preemptive vaccines and therapeutic monoclonal antibodies that can be evaluated before the emergence of human-adapted H5N1 strains.

### Definitions

Unless defined otherwise, technical and scientific terms used herein have the same meaning as commonly understood by one of ordinary skill in the art to which this invention belongs. See, e.g., Singleton P and Sainsbury D., in Dictionary of Microbiology and Molecular Biology 3rd ed., J. Wiley & Sons, Chichester, New York, 2001; and Fields Virology 4th ed., Knipe D. M. and Howley P. M. eds, Lippincott Williams & Wilkins, Philadelphia 2001.

The transitional term “comprising” is synonymous with “including,” “containing,” or “characterized by,” is inclusive or open-ended and does not exclude additional, unrecited elements or method steps.

The transitional phrase “consisting of” excludes any element, step, or ingredient not specified in the claim, but does not exclude additional components or steps that are unrelated to the invention such as impurities ordinarily associated therewith.

The transitional phrase “consisting essentially of” limits the scope of a claim to the specified materials or steps and those that do not materially affect the basic and novel characteristic(s) of the claimed invention.

As used in this specification and the appended claims, the singular forms “a”, “an”, and “the” include plural referents unless the context clearly dictates otherwise. Thus, for example, reference to “a virus” includes a plurality of viruses; reference to a “host cell” includes mixtures of host cells, and the like.

The terms “nucleic acid”, “polynucleotide”, “polynucleotide sequence” and “nucleic acid sequence” refer to single-stranded or double-stranded deoxyribonucleotide or ribonucleotide polymers, chimeras or analogues thereof, or a character string representing such, depending on context. As used herein, the term optionally includes polymers of analogs of naturally occurring nucleotides having the essential nature of natural nucleotides in that they hybridize to single-stranded nucleic acids in a manner similar to naturally occurring nucleotides (e.g., polyamide nucleic acids). Unless otherwise indicated, a particular nucleic acid sequence of this invention optionally encompasses complementary sequences in addition to the sequence explicitly indicated. From any specified polynucleotide sequence, either the given nucleic acid or the complementary polynucleotide sequence (e.g., the complementary nucleic acid) can be determined.

The term “nucleic acid” or “polynucleotide” also encompasses any physical string of monomer units that can be corresponded to a string of nucleotides, including a polymer of nucleotides (e.g., a typical DNA or RNA polymer), PNAs, modified oligonucleotides (e.g., oligonucleotides comprising bases that are not typical to biological RNA or DNA in solution, such as 2′-O-methylated oligonucleotides), and the like. A nucleic acid can be e.g., single-stranded or double-stranded.

A “subsequence” is any portion of an entire sequence, up to and including the complete sequence. Typically, a subsequence comprises less than the full-length sequence.

The phrase “substantially identical”, in the context of two nucleic acids or polypeptides (e.g., DNAs encoding a HA molecule, or the amino acid sequence of a HA molecule) refers to two or more sequences or subsequences that have at least about 90%, preferably 91%, most preferably 92%, 93%, 94%, 95%, 96%, 97%, 98%, 98.5%, 99%, 99.1%, 99.2%, 99.3%, 99.4%, 99.5%, 99.6%, 99.7%, 99.8%, 99.9% or more nucleotide or amino acid residue identity, when compared and aligned for maximum correspondence, as measured using a sequence comparison algorithm or by visual inspection.

The term “variant” with respect to a polypeptide refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g, replacement of a glycine with a tryptophan. Analogous minor variation can also include amino acid deletion or; insertion, or both. Guidance in determining which amino acid residues can be substituted, inserted, or deleted without eliminating biological or immunological activity can be found using computer programs well known in the art, for example, DNASTAR software. Examples of conservative substitutions are also described herein.

The term “gene” is used broadly to refer to any nucleic acid associated with a biological function. Thus, genes include coding sequences and/or the regulatory sequences required for their expression. The term “gene” applies to a specific genomic sequence, as well as to a cDNA or an mRNA encoded by that genomic sequence.

Genes also include non-expressed nucleic acid segments that, for example, form recognition sequences for other proteins. Non-expressed regulatory sequences include “promoters” and “enhancers”, to which regulatory proteins such as transcription factors bind, resulting in transcription of adjacent or nearby sequences. A “tissue specific” promoter or enhancer is one that regulates transcription in a specific tissue type or cell type, or types.

“Expression of a gene” or “expression of a nucleic acid” typically means transcription of DNA into RNA (optionally including modification of the RNA, e.g., splicing) or transcription of RNA into mRNA, translation of RNA into a polypeptide (possibly including subsequent modification of the polypeptide, e.g., post-translational modification), or both transcription and translation, as indicated by the context.

An “open reading frame” or “ORF” is a possible translational reading frame of DNA or RNA (e.g., of a gene), which is Capable of being translated into a polypeptide. That is, the reading frame is not interrupted by stop codons. However, it should be noted that the term ORF does not necessarily indicate that the polynucleotide is, in fact, translated into a polypeptide.

The term “vector” refers to the means by which a nucleic acid can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present invention are plasmids.

An “expression vector” is a vector, such as a plasmid that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer.

A “bi-directional expression vector” is characterized by two alternative promoters oriented in the opposite direction relative to a nucleic acid situated between the two promoters, such that expression can be initiated in both orientations resulting in, e.g., transcription of both plus (+) or sense strand, and negative (−) or antisense strand RNAs.

An “amino acid sequence” is a polymer of amino acid residues (a protein, polypeptide, etc.) or a character string representing an amino acid polymer, depending on context.

A “polypeptide” is a polymer comprising two or more amino acid residues (e.g., a peptide or a protein). The polymer can optionally comprise modifications such as glycosylation or the like. The amino acid residues of the polypeptide can be natural or non-natural and can be unsubstituted, unmodified, substituted or modified.

In the context of the invention, the term “isolated” refers to a biological material, such as a virus, a nucleic acid or a protein, which is substantially free from components that normally accompany or interact with it in its naturally occurring environment. The isolated biological material optionally comprises additional material not found with the biological material in its natural environment, e.g., a cell or wild-type virus.

For example, if the material is in its natural environment, such as a cell, the material can have been placed at a location in the cell (e.g., genome or genetic element) not native to such material found in that environment. For example, a naturally occurring nucleic acid (e.g., a coding sequence, a promoter, an enhancer, etc.) becomes isolated if it is introduced by non-naturally occurring means to a locus of the genome (e.g., a vector, such as a plasmid or virus vector, or amplicon) not native to that nucleic acid. Such nucleic acids are also referred to as ‘heterologous” nucleic acids. An isolated virus, for example, is in an environment (e.g., a cell culture system, or purified from cell culture) other than the native environment of wild-type virus (e.g., the intestinal or respiratory tract of an infected individual).

The term “recombinant” indicates that the material (e.g., a nucleic acid or protein) has been artificially or synthetically (non-naturally) altered by human intervention. The alteration can be performed on the material within, or removed from, its natural environment or state. Specifically, e.g., an influenza virus is recombinant when it is produced by the expression of a recombinant nucleic acid. For example, a “recombinant nucleic acid” is one that is made by recombining nucleic acids, e.g., during cloning, or other procedures, or by chemical or other mutagenesis; and a “recombinant polypeptide” or “recombinant protein” is a polypeptide or protein which is produced by expression of a recombinant nucleic acid.

The term “introduced” when referring to a heterologous or isolated nucleic acid refers to the incorporation of a nucleic acid into a eukaryotic or prokaryotic cell where the nucleic acid can be incorporated into the genome of the cell (e.g., chromosome, plasmid, or mitochondrial DNA), converted into an autonomous replicon, or transiently expressed (e.g., transfected mRNA). The term includes such methods as “transfection”, “transformation” and “transduction.” In the context of the invention a variety of methods can be employed to introduce nucleic acids into cells, including electroporation, calcium phosphate precipitation, lipid mediated transfection (lipofection), etc.

The term “host cell” means a cell that contains a heterologous nucleic acid, such as a vector or a virus, and supports the replication and/or expression of the nucleic acid. Host cells can be prokaryotic cells such as E. coli, or eukaryotic cells such as yeast, insect, amphibian, avian or mammalian cells, including human cells. Exemplary host cells can include, e.g., Vero (African green monkey kidney) cells, BHK (baby hamster kidney) cells, primary chick kidney (PCK) cells, Madin-Darby Canine Kidney (MDCK) cells, Madin-Darby Bovine Kidney (MDBK) cells, 293 cells (e.g., 293T cells), and COS cells (e.g., COS1, COS7 cells), etc.

An “immunologically effective amount” of influenza virus is an amount sufficient to enhance an individual's (e.g., a human's) own immune response against a subsequent exposure to influenza virus. Levels of induced immunity can be monitored, e.g., by measuring amounts of neutralizing secretory and/or serum antibodies, e.g., by plaque neutralization, complement fixation, enzyme-linked immunosorbent, or microneutralization assay.

A “protective immune response” against influenza virus refers to an immune response exhibited by an individual (e.g., a human) that is protective against disease when the individual is subsequently exposed to and/or infected with wild-type influenza virus. In some instances, the wild-type (e.g., naturally circulating) influenza virus can still cause infection, but it cannot cause a serious infection. Typically, the protective immune response results in detectable levels of host engendered serum and secretory antibodies that are capable of neutralizing virus of the same strain and/or subgroup (and possibly also of a different, non-vaccine strain and/or subgroup) in vitro and in vivo.

As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into a Fab′ monomer. The Fab′ monomer is essentially a Fab with part of the hinge region (see Fundamental Immunology, W. E. Paul, ed., Raven Press, N.Y. (1999) for a more detailed description of other antibody fragments). While various antibody fragments are defined in terms of e digestion of an intact antibody, one of skill will appreciate that such Fab′ fragments may be synthesized de novo either chemically or by utilizing recombinant DNA methodology. Thus, the term antibody, as used herein, includes antibodies or fragments either produced by the modification of whole antibodies or synthesized de novo using recombinant DNA methodologies. Antibodies include, e.g., polyclonal antibodies, monoclonal antibodies, multiple or single chain antibodies, including single chain Fv (sFv or scFv) antibodies in which a variable heavy and a variable light chain are joined together (directly or through a peptide linker) to form a continuous polypeptide, and humanized or chimeric antibodies.

### List of Standard Amino Acid Abbreviations

| Amino Acid [symbol] | 3-Letter [symbol] | 1-Letter [symbol] |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### Influenza Viruses

Influenza A is an enveloped negative single-stranded RNA virus that infects a wide range of avian and mammalian species. The influenza A viruses are classified into serologically-defined antigenic subtypes of the hemagglutinin (HA) and neuraminidase (NA) major surface glycoproteins (WHO Memorandum 1980 Bull WHO 58:585-591). The nomenclature meets the requirement for a simple system that can be used by all countries and it has been in effect since 1980. It is based on data derived from double immunodiffusion (DID) reactions involving hemagglutinin and neuraminidase antigens.

Double immunodiffusion (DID) tests are performed as described previously (Schild, G C et al. 1980 Arch Virol 63:171-184). Briefly, tests are carried out in agarose gels (HGT agarose, 1% phosphate-buffered saline, pH 7.2 containing 0.01 percent sodium azide). Preparations of purified virus particles containing 5-15 mg virus protein per ml (or an HA titer with chick erythrocytes of 10^{5.5}-10^{6.5} hemagglutinin units per 0.25 ml) are added in 5-10 μl volumes to wells in the gel. The virus particles are disrupted in the wells by the addition of sarcosyl detergent NL97, 1 percent final concentration). The precipitin reactions are either photographed without staining or, the gels are dried and stained with Coomassie Brilliant Blue.

The DID test, when performed using hyperimmune sera specific to one or other of the antigens, provides a valuable method for comparing antigenic relationships. Similarities between antigens are detected as lines of common precipitin, whereas the existence of variation between antigens is revealed by spurs of precipitin when different antigens are permitted to diffuse radically inwards toward a single serum. Based on the results of DID tests on influenza A viruses from all species, the H antigens can be grouped into 16 subtypes as indicated in Table 2).

TABLE 2
Hemagglutinin subtypes of influenza A viruses isolated from
humans, lower mammals and birds

| | Species of Origin^{a} | | | |
|---|---|---|---|---|
| Subtypes | Humans | Swine | Horses | Birds |
| H1^{b} | PR/8/34 | Sw/Ia/15/30 | — | Dk/Alb/35/76 |
| H2 | Sing/1/57 | — | — | Dk/Ger/1215/73 |
| H3 | HK/1/68 | Sw/Taiwan/70 | Eq/Miami/ | Dk/Ukr/1/63 |
| | | | 1/63 | |
| H4 | — | — | — | Dk/Cz/56 |
| H5 | — | — | — | Tern/S.A./61 |
| H6 | — | — | — | Ty/Mass/3740/65 |
| H7 | — | — | Eq/ | FPV/Dutch/27 |
| | | | Prague/ | |
| | | | 1/56 | |
| H8 | — | — | — | Ty/Ont/6118/68 |
| H9 | — | — | — | Ty/Wis/1/66 |
| H10 | — | — | — | Ck/Ger/N/49 |
| H11 | — | — | — | Dk/Eng/56 |
| H12 | — | — | — | Dk/Alb/60/76 |
| H13 | — | — | — | Gull/MD/704/77 |
| H14 | — | — | — | Dk/Gurjev/263/82 |
| H15 | — | — | — | Dk/Austral/3431/83 |
| H16 | — | — | — | A/Black-headed |
| | — | — | — | Gull/Sweden/5/99 |

^{a}The reference strains of influenza viruses, or the first isolates from that species, are presented.
^{b}Current subtype designation. From WHO Memorandum 1980 Bull WHO 58: 585-591.

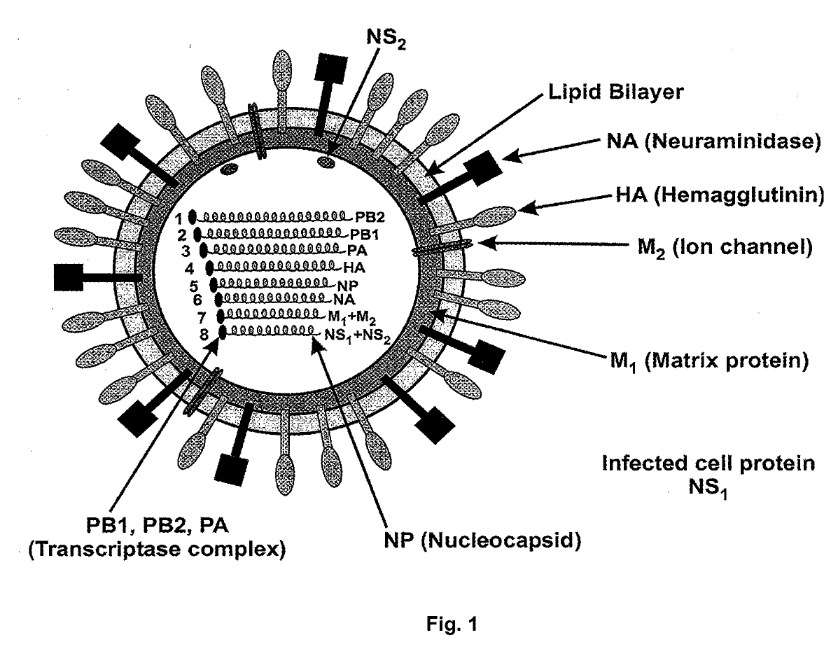

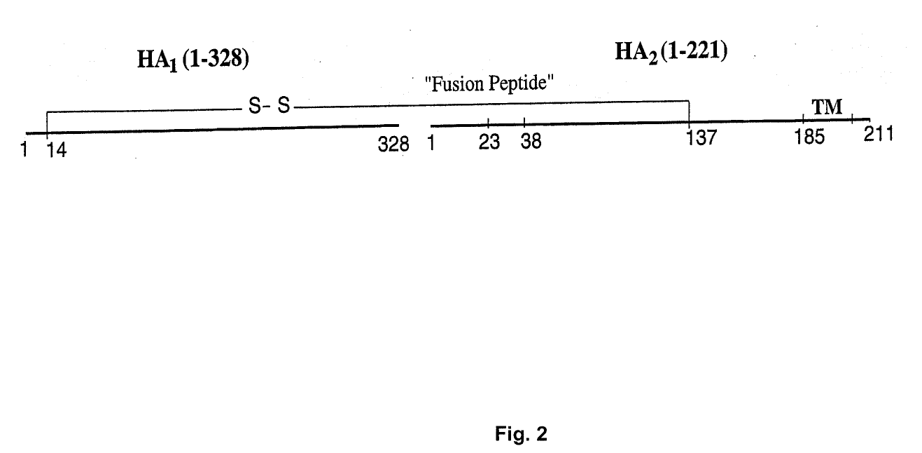

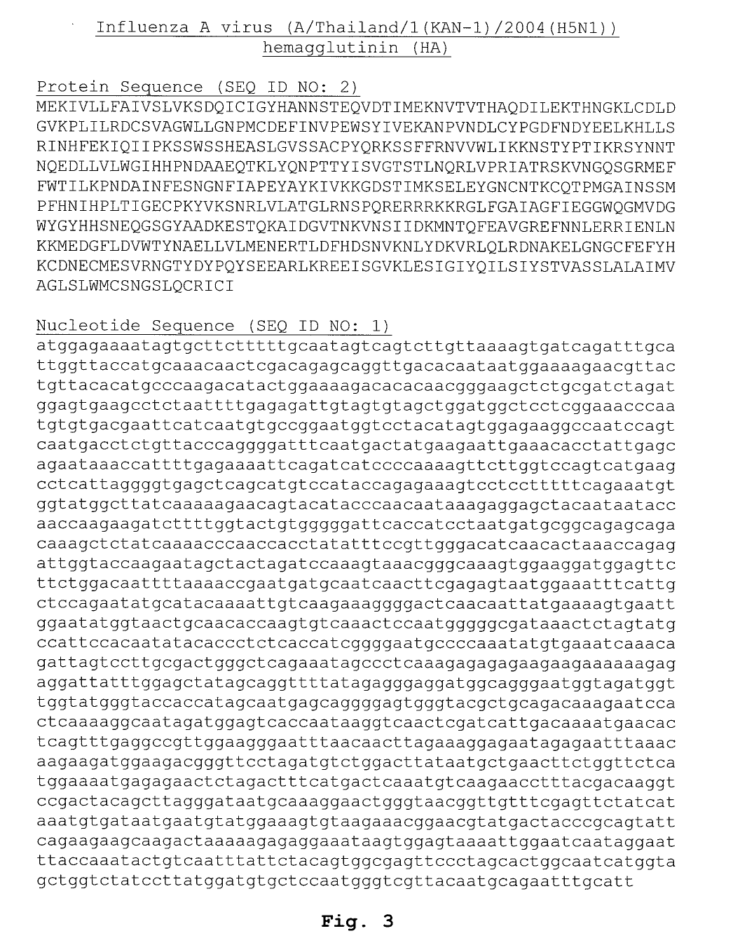

The influenza A genome consists of eight single-stranded negative-sense RNA molecules (FIG. 1). Three types of integral membrane protein-hemagglutinin (HA), neuraminidase (NA), and small amounts of the M2 ion channel protein-are inserted through the lipid bilayer of the viral membrane. The virion matrix protein M1 is thought to underlie the lipid bilayer but also to interact with the helical ribonucleoproteins (RNPs). Within the envelope are eight segments of single-stranded genome RNA (ranging from 2341 to 890 nucleotides) contained in the form of an RNP. Associated with the RNPs are small amounts of the transcriptase complex, consisting of the proteins PB1, PB2, and PA. The coding assignments of the eight RNA segments are also illustrated in FIG. 1. HA and NA are encoded on separate RNA molecules. HA is involved in viral attachment to terminal sialic acid residues on host cell glycoproteins and glycolipids. After viral entry into an acidic endosomal compartment of the cell, HA is also involved in fusion with the cell membrane, which results in the intracellular release of the virion contents. HA is synthesized as an HA₀ precursor that forms noncovalently bound homotrimers on the viral surface. The HA₀ precursor is cleaved by host proteases at a conserved arginine residue to create two subunits, HA₁ and HA₂, which are associated by a single disulfide bond (FIG. 2). This cleavage event is required for productive infection. NA cleaves terminal sialic acid residues of influenza A cellular receptors and is involved in the release and spread of mature virions; it may also contribute to initial viral entry.

### Antigenic Shift and Drift

The segmentation of the influenza A genome facilitates reassortment among strains, when two or more strains infect the same cell. Reassortment can yield major genetic changes, referred to as antigenic shifts. In contrast, antigenic drift is the accumulation of viral strains with minor genetic changes, mainly amino acid substitutions in the HA and NA proteins. Influenza A nucleic acid replication by the virus-encoded RNA-dependent RNA polymerase complex is relatively error-prone, and these point mutations (˜1/10⁴ bases per replication cycle) in the RNA genome are the major source of genetic variation for antigenic drift.

Selection favors human influenza A strains with antigenic drift and shift involving the HA and NA proteins because these strains are able to evade neutralizing antibody from prior infection or vaccination. This selection allows viral reinfection with a new subtype (shift) or the same viral subtype (drift). Antigenic shifts caused three of the major influenza A pandemics in the twentieth century, including the 1918 H1N1 (Spanish flu), the 1957H2N2 (Asian flu) and the 1968H3N2 (Hong Kong flu) outbreaks. Antigenic drift accounts for the annual nature of flu epidemics. It also explains the reduced efficacy of influenza A vaccination, which is based on neutralizing antibody: For a particular subtype, if the amino acid sequence of the HA protein used in vaccination does not match that encountered during the epidemic, antibody neutralization may be ineffective.

### Determinants of Tissue Tropism

The binding specificity of influenza A HA for integral glycoproteins or glycolipids on the host cell surface appears to be a key determinant of whether a particular influenza A subtype can infect humans. Avian influenza viruses, such as the H5N1 subtype, preferentially bind to cell surface receptors that consist of terminal sialic acid with a 2-3 linkage (NeurAc(α2-3)Gal) to a penultimate galactose residue of glycoproteins or glycolipids. In contrast, human lineage viruses, including the early isolates from the 1918, 1957 and 1968 pandemics, bind to receptors in which these terminal sialy-galactosyl residues have a 2-6 linkage (NeurAc(α2-6)Gal). The tracheal epithelia of birds and humans mainly express influenza A receptors with a 2-3 linkage and 2-6 linkage of sialic acid, respectively.

### Vectors Promoters and Expression Systems

The present invention includes recombinant constructs incorporating one or more of the nucleic acid sequences described herein. Such constructs optionally include a vector, for example, a plasmid, a cosmid, a phage, a virus, a bacterial artificial chromosome (BAC), a yeast artificial chromosome (YAC), etc., into which one or more of the polynucleotide sequences of the invention, e.g., comprising an avian H5 framework comprising at least one mutation that changes receptor specificity as described herein, or a subsequence thereof etc., has been inserted, in a forward or reverse orientation. For example, the inserted nucleic acid can include a viral chromosomal sequence or cDNA including all or part of at least one of the polynucleotide sequences of the invention. In one embodiment, the construct further comprises regulatory sequences, including, for example, a promoter, operably linked to the sequence. Large numbers of suitable vectors and promoters are known to those of skill in the art, and are commercially available.

The polynucleotides of the present invention can be included in any one of a variety of vectors suitable for generating sense or antisense RNA, and optionally, polypeptide (or peptide) expression products (e.g., a hemagglutinin molecule of the invention, or fragments thereof). Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others (e.g., pCMV/R) (Barouch et al. 2005 J Virol 79:8828-8834). Any vector that is capable of introducing genetic material into a cell, and, if replication is desired, which is replicable in the relevant host can be used.

In an expression vector, the HA polynucleotide sequence of interest is physically arranged in proximity and orientation to an appropriate transcription control sequence (e.g., promoter, and optionally, one or more enhancers) to direct mRNA synthesis. That is, the polynucleotide sequence of interest is operably linked to an appropriate transcription control sequence. Examples of such promoters include: LTR or SV40 promoter, E. coli lac or trp promoter, phage lambda P_{L} promoter, and other promoters known to control expression of genes in prokaryotic or eukaryotic cells or their viruses.

A variety of promoters are suitable for use in expression vectors for regulating transcription of influenza virus genome segment sequences. In certain embodiments, the cytomegalovirus (CMV) DNA dependent RNA Polymerase II (Pol II) promoter is utilized. If desired, e.g., for regulating conditional expression, other promoters can be substituted which induce RNA transcription under the specified conditions, or in the specified tissues or cells. Numerous viral and mammalian, e.g., human promoters are available, or can be isolated according to the specific application contemplated. For example, alternative promoters obtained from the genomes of animal and human viruses include such promoters as the adenovirus (such as Adenovirus 2), papilloma virus, hepatitis-B virus, polyoma virus, and Simian Virus 40 (SV40), and various retroviral promoters. Mammalian promoters include, among many others, the actin promoter, immunoglobulin promoters, heat-shock promoters, and the like.

Transcription is optionally increased by including an enhancer sequence. Enhancers are typically short, e.g., 10-500 bp, cis-acting DNA elements that act in concert with a promoter to increase transcription. Many enhancer sequences have been isolated from mammalian genes (hemoglobin, elastase, albumin, alpha-fetoprotein, and insulin), and eukaryotic cell viruses. The enhancer can be spliced into the vector at a position 5′ or 3′ to the heterologous coding sequence, but is typically inserted at a site 5′ to the promoter. Typically, the promoter, and if desired, additional transcription enhancing sequences are chosen to optimize expression in the host cell type into which the heterologous DNA is to be introduced. Optionally, the amplicon can also contain a ribosome binding site or an internal ribosome entry site (IRES) for translation initiation.

The vectors of the invention also favorably include sequences necessary for the termination of transcription and for stabilizing the mRNA, such as a polyadenylation site or a terminator sequence. Such sequences are commonly available from the 3′ and, occasionally 5′, untranslated regions of eukaryotic or viral DNAs or cDNAs. In one embodiment, the bovine growth hormone terminator can provide a polyadenylation signal sequence.

In addition, as described above, the expression vectors optionally include one or more selectable marker genes to provide a phenotypic trait for selection of transformed host cells, in addition to genes previously listed, markers such as dihydrofolate reductase or kanamycin resistance are suitable for selection in eukaryotic cell culture.

The vector containing the appropriate nucleic acid sequence as described above, as well as an appropriate promoter or control sequence, can be employed to transform a host cell permitting expression of the protein. While the vectors of the invention can be replicated in bacterial cells, frequently it will be desirable to introduce them into mammalian cells, e.g., Vero cells, BHK cells, MDCK cells, 293 cells, COS cells, or the like, for the purpose of expression.

### Additional Expression Elements

Most commonly, the genome segment encoding the influenza virus HA protein includes any additional sequences necessary for its expression, including translation into a functional viral protein. In other situations, a minigene, or other artificial construct encoding the viral proteins, e.g., an HA protein, can be employed. Again, in such case, it is often desirable to include specific initiation signals that aid in the efficient translation of the heterologous coding sequence. These signals can include, e.g., the ATG initiation codon and adjacent sequences. To insure translation of the entire insert, the initiation codon is inserted in the correct reading frame relative to the viral protein. Exogenous transcriptional elements and initiation codons can be of various origins, both natural and synthetic. The efficiency of expression can be enhanced by the inclusion of enhancers appropriate to the cell system in use.

If desired, polynucleotide sequences encoding additional expressed elements, such as signal sequences, secretion or localization sequences, and the like can be incorporated into the vector, usually, in-frame with the polyoucleotide sequence of interest, e.g., to target polypeptide expression to a desired cellular compartment, membrane, or organelle, or to direct polypeptide secretion to the periplasmic space or into the cell culture media. Such sequences are known to those of skill, and include secretion leader peptides, organelle targeting sequences (e.g., nuclear localization sequences, ER retention signals, mitochondrial transit sequences), membrane localization/anchor sequences (e.g., stop transfer sequences, GPI anchor sequences), and the like.

Where translation of a polypeptide encoded by a nucleic acid sequence of the invention is desired, additional translation specific initiation signals can improve the efficiency of translation. These signals can include, e.g., an ATG initiation codon and adjacent sequences, an IRES region, etc. In some cases, for example, full-length cDNA molecules or chromosomal segments including a coding sequence incorporating, e.g., a polynucleotide sequence of the invention (e.g., as in the sequences herein), a translation initiation codon and associated sequence elements are inserted into the appropriate: expression vector simultaneously with the polynucleotide sequence of interest. In such; cases, additional translational control signals frequently are not required. However, in cases where only a polypeptide coding sequence, or a portion thereof, is inserted, exogenous translational control signals, including, e.g., an ATG initiation codon is often provided for expression of the relevant sequence. The initiation codon is put in the correct reading frame to ensure transcription of the polynucleotide sequence of interest. Exogenous transcriptional elements and initiation codons can be of various origins, both natural and synthetic. The efficiency of expression can be enhanced by the inclusion of enhancers appropriate to the cell system in use.

### Cell Culture and Expression Hosts

The present invention also relates to host cells that are introduced (transduced, transformed or transfected) with vectors of the invention, and the production of polypeptides of the invention by recombinant techniques. Host cells are genetically engineered (i.e., transduced, transformed or transfected) with a vector, such as an expression vector, of this invention. As described above, the vector can be in the form of a plasmid, a viral particle, a phage, etc. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; or insect cells such as Drosophila and Spodoptera frugiperda.

Commonly, mammalian cells are used to culture the HA molecules of the invention. Suitable host cells for the replication of the HA sequences herein include, e.g., Vero cells, BHK cells, MDCK cells, 293 cells and COS cells, including 293T cells, COS7 cells or the like. Typically, cells are cultured in a standard commercial culture medium, such as Dulbecco's modified Eagle's medium supplemented with serum (e.g., 10% fetal bovine serum), or in serum free medium, under controlled humidity and CO₂ concentration suitable for maintaining neutral buffered pH (e.g., at pH between 7.0 and 7.2). Optionally, the medium contains antibiotics to prevent bacterial growth, e.g., penicillin, streptomycin, etc., and/or additional nutrients, such as L-glutamine, sodium pyruvate, non-essential amino acids, additional supplements to promote favorable growth characteristics, e.g., trypsin, B-mercaptoethanol, and the like.

The engineered host cells can be cultured in conventional nutrient media modified as appropriate for activating promoters, selecting transformants, or amplifying the inserted polynucleotide sequences. The culture conditions, such as temperature, pH and the like, are typically those previously used with the particular host cell selected for expression, and will be apparent to those skilled in the art and in the references cited herein, including Sambrook et al., Molecular Cloning-A Laboratory Manual (3rd Ed.), Vol. 1-3, Cold Spring Harbor Laboratory, Cold Spring Harbor, N.Y., 2001 (“Sambrook”) and Current Protocols in Molecular Biology, F. M. Ausubel et al., eds., Current Protocols, a joint venture between Greene Publishing Associates, Inc. and John Wiley & Sons, Inc. (“Ausubel”) Additionally, variations in such procedures adapted to the present invention are readily determined through routine experimentation and will be familiar to those skilled in the art.

In mammalian host cells, a number of expression systems, such as viral-based systems, can be utilized. In cases where an adenovirus is used as an expression vector, a coding sequence is optionally ligated into an adenovirus transcription/translation complex consisting of the late promoter and tripartite leader sequence. Insertion in a nonessential E1 or E3 region of the viral genome will result in a viable virus capable of expressing the polypeptides of interest in infected host cells. In addition, transcription enhancers, such as the rous sarcoma virus (RSV) enhancer, can be used to increase expression in mammalian host cells.

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing, which cleaves a precursor form into a mature form, of the protein is sometimes important for correct insertion, folding and/or function. Additionally proper location within a host cell (e.g., on the cell surface) is also important. Different host cells such as COS, CHO, BHK, MDCK, 293, 293T, COS7, etc. have specific cellular machinery and characteristic mechanisms for such post translational activities and can be chosen to ensure the correct modification and processing of the current introduced, foreign protein.

For long-term, high-yield production of recombinant proteins encoded by, or having subsequences encoded by, the polynucleotides of the invention, stable expression systems are optionally used. For example, cell lines, stably expressing a polypeptide of the invention, are transfected using expression vectors that contain viral origins of replication or endogenous expression elements and a selectable marker gene. For example, following the introduction of the vector, cells are allowed to grow for 1-2 days in an enriched media before they are switched to selective media. The purpose of the selectable marker is to confer resistance to selection, and its presence allows growth and recovery of cells that successfully express the introduced sequences. Thus, resistant clumps of stably transformed cells, e.g., derived from single cell type, can be proliferated using tissue culture techniques appropriate to the cell type.

Host cells transformed with a nucleotide sequence encoding a polypeptide of the invention are optionally cultured under conditions suitable for the expression and recovery of the encoded protein from cell culture. The cells expressing said protein can be sorted, isolated and/or purified. The protein or fragment thereof produced by a recombinant cell can be secreted, membrane-bound, or retained intracellularly, depending on the sequence (e.g., depending upon fusion proteins encoding a membrane retention signal or the like) and/or the vector used.

Expression products corresponding to the nucleic acids of the invention can also be produced in non-animal cells such as plants, yeast, fungi, bacteria and the like. Refer to Sambrook and Ausubel, supra.

In bacterial systems, a number of expression vectors can be selected depending upon the use intended for the expressed product. For example, when large quantities of a polypeptide or fragments thereof are needed for the production of antibodies, vectors that direct high-level expression of fusion proteins that are readily purified are favorably employed. Such vectors include, but are not limited to, multifunctional E. coli cloning and expression vectors such as BLUESCRIPT (Stratagene), in which the coding sequence of interest, e.g., sequences comprising those found herein, etc., can be ligated into the vector in-frame with sequences for the amino-terminal translation initiating methionine and the subsequent 7 residues of beta-galactosidase producing a catalytically active beta galactosidase fusion protein; pIN vectors; pET vectors; and the like. Similarly, in the yeast Saccharomyces cerevisiae a number of vectors containing constitutive or inducible promoters such as alpha factor, alcohol oxidase and PGH can be used for production of the desired expression products.

### Nucleic Acid Hybridization

Comparative hybridization can be used to identify nucleic acids of the invention, including conservative variations of nucleic acids of the invention. This comparative hybridization method is a preferred method of distinguishing nucleic acids of the invention. In addition, target nucleic acids which hybridize to the nucleic acids represented by sequences under high, ultra-high and ultra-ultra-high stringency conditions are features of the invention. Examples of such nucleic acids include those with one or a few silent or conservative nucleic acid substitutions as compared to a given nucleic acid sequence.

A test target nucleic acid is said to specifically hybridize to a probe nucleic acid when it hybridizes at least one-half as well to the probe as to the perfectly matched complementary target, i.e., with a signal to noise ratio at least one-half as high as hybridization of the probe to the target under conditions in which the perfectly matched probe binds to the perfectly matched complementary target with a signal to noise ratio that is at least about 5×-10× as high as that observed for hybridization to any of the unmatched target nucleic acids.

Nucleic acids “hybridize” when they associate, typically in solution. Nucleic acids hybridize due to a variety of well-characterized physico-chemical forces, such as hydrogen bonding, solvent exclusion, base stacking and the like. Numerous protocols for nucleic acid hybridization are well known in the art. An extensive guide to the hybridization of nucleic acids is found in Sambrook and Ausubel, supra.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of stringent wash conditions comprises a 0.2×SSC wash at 65° C. for 15 minutes. Often the high stringency wash is preceded by a low stringency wash to remove background probe signal. An example low stringency wash is 2×SSC at 40° C. for 15 minutes. In general, a signal to noise ratio of 5× (or higher) than that observed for an unrelated probe in the particular hybridization assay indicates detection of a specific hybridization.

After hybridization, unhybridized nucleic acids can be removed by a series of washes, the stringency of which can be adjusted depending upon the desired results. Low stringency washing conditions (e.g., using higher salt and lower temperature) increase sensitivity, but can produce nonspecific hybridization signals and high background signals. Higher stringency conditions (e.g., using lower salt and higher temperature that is closer to the Tm) lower the background signal, typically with primarily the specific signal remaining.

“Stringent hybridization wash conditions” in the context of nucleic acid hybridization experiments such as Southern and northern hybridizations are sequence dependent, and are different under different environmental parameters. Stringent hybridization and wash conditions can easily be determined empirically for any test nucleic acid. For example, in determining highly stringent hybridization and wash conditions, the hybridization and wash conditions are gradually increased (e.g., by increasing temperature, decreasing salt concentration, increasing detergent concentration and/or increasing the concentration of organic solvents such as formalin in the hybridization or wash), until a selected set of criteria is met. For example, the hybridization and wash conditions are gradually increased until a probe binds to a perfectly matched complementary target with a signal to noise ratio that is at least 5× as high as that observed for hybridization of the probe to an unmatched target.

In general, a signal to noise ratio of at least 2× (or higher, e.g., at least 5×, 10×, 20×, 50×, 100×, or more) than that observed for an unrelated probe in the particular hybridization assay indicates detection of a specific hybridization. Detection of at least stringent hybridization between two sequences in the context of the present invention indicates relatively strong structural similarity to, e.g., the nucleic acids of the present invention.

“Very stringent” conditions are selected to be equal to the thermal melting point (Tm) for a particular probe. The Tm is the temperature (under defined ionic strength and pH) at which 50% of the test sequence hybridizes to a perfectly matched probe. For the purposes of the present invention, generally, “highly stringent” hybridization and wash conditions are selected to be about 5° C. lower than the Tm for the specific sequence at a defined ionic strength and pH (as noted below, highly stringent conditions can also be referred to in comparative terms). Target sequences that are closely related or identical to the nucleotide sequence of interest (e.g., “probe”) can be identified under stringent or highly stringent conditions. Lower stringency conditions are appropriate for sequences that are less complementary.

“Ultra high-stringency” hybridization and wash conditions are those in which the stringency of hybridization and wash conditions are increased until the signal to noise ratio for binding of the probe to the perfectly matched complementary target nucleic acid is at least 10× as high as that observed for hybridization to any unmatched target nucleic acids. A target nucleic acid which hybridizes to a probe under such conditions, with a signal to noise ratio of at least one-half that of the perfectly matched complementary target nucleic acid is said to bind to the probe under ultra-high stringency conditions.

In determining stringent or highly stringent hybridization (or even more stringent hybridization) and wash conditions, the hybridization and wash conditions are gradually increased (e.g., by increasing temperature, decreasing salt concentration, increasing detergent concentration and/or increasing the concentration of organic solvents, such as formamide, in the hybridization or wash), until a selected set of criteria are met. For example, the hybridization and wash conditions are gradually increased until a probe comprising one or more polynucleotide sequences of the invention, e.g., sequences or subsequences selected from those given herein and/or complementary polynucleotide sequences, binds to a perfectly matched complementary target (again, a nucleic acid comprising one or more nucleic acid sequences or subsequences selected from those given herein and/or complementary polynucleotide sequences thereof), with a signal to noise ratio that is at least 2× (and optionally 5×, 10×, or 100× or more) as high as that observed for hybridization of the probe to an unmatched target (e.g., a polynucleotide sequence comprising one or more sequences or subsequences selected from known influenza sequences present in public databases such as GenBank at the time of filing, and/or complementary polynucleotide sequences thereof), as desired.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or 500× or more as high as that observed for hybridization to any unmatched target nucleic acids. The particular signal will depend on the label used in the relevant assay, e.g., a fluorescent label, a calorimetric label, a radioactive label, or the like. A target nucleic acid which hybridizes to a probe under such conditions, with a signal to noise ratio of at least one-half that of the perfectly matched complementary target nucleic acid, is said to bind to the probe under ultra-ultra-high stringency conditions.

### Cloning, Mutagenesis and Expression of Biomolecules of Interest

General texts which describe molecular biological techniques, which are applicable to the present invention, such as cloning, mutation, cell culture and the like include Sambrook and Ausubel, supra These texts describe mutagenesis, the use of vectors, promoters and many other relevant topics related to, e.g., the generation of HA molecules, etc.

Various types of mutagenesis are optionally used in the present invention, e.g., to produce and/or isolate, e.g., novel or newly isolated HA molecules and/or to further modify/mutate the polypeptides (e.g., HA molecules) of the invention. They include but are not limited to site-directed, random point mutagenesis, mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. In one embodiment, mutagenesis can be guided by known information of the naturally occurring molecule or altered or mutated naturally occurring molecule, e.g., sequence, sequence comparisons, physical properties, crystal structure or the like.

Oligonucleotides, e.g., for use in mutagenesis of the present invention, e.g., mutating the HA molecules of the invention, or altering such, are typically synthesized chemically according to the solid phase phosphoramidite triester method described by Beaucage and Caruthers 1981 Tetrahedron Letts 22:1859-1862, e.g., using an automated synthesizer, as described in Needham-VanDevanter et al. 1984 Nucleic Acids Res 12:6159-6168. In addition, essentially any nucleic acid can be custom or standard ordered from any of a variety of commercial sources.

The present invention also relates to host cells and organisms comprising an HA molecule or other polypeptide and/or nucleic acid of the invention or such HA or other sequences within various vectors, etc. Host cells are genetically engineered (e.g., transformed, transduced or transfected) with the vectors of this invention, which can be, for example, a cloning vector or an expression vector. The vector can be, for example, in the form of a plasmid, a bacterium, a virus, a naked polynucleotide, or a conjugated polynucleotide. The vectors are introduced into cells and/or microorganisms by standard methods including electroporation, infection by viral vectors, high velocity ballistic penetration by small particles with the nucleic acid either within the matrix of small beads or particles, or on the surface. Sambrook and Ausubel, supra, provide a variety of appropriate transformation methods.

Several well-known methods of introducing target nucleic acids into bacterial cells are available, any of which can be used in the present invention. These include: fusion of the recipient cells with bacterial protoplasts containing the DNA, electroporation, projectile bombardment, and infection with viral vectors, etc. Bacterial cells can be used to amplify the number of plasmids containing DNA constructs of this invention. The bacteria are grown to log phase and the plasmids within the bacteria can be isolated by a variety of methods known in the art (see, for instance, Sambrook). In addition, a plethora of kits are commercially available for the purification of plasmids from bacteria. The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Sambrook and Ausubel (at supra). A catalogue of Bacteria and Bacteriophages useful for cloning is provided, e.g., on the world-wide-web at ATCC.org. Additional basic procedures for sequencing, cloning and other aspects of molecular biology and underlying theoretical considerations are also found in Watson et al. (1992) Recombinant DNA Second Edition Scientific American Books, NY.

### Polypeptide Production and Recovery

In some embodiments, following transduction of a suitable host cell line or strain and growth of the host cells to an appropriate cell density, a selected promoter is induced by appropriate means (e.g., temperature shift or chemical induction) and cells are cultured for an additional period. In some embodiments, a secreted polypeptide product, e.g., a HA polypeptide as in a secreted fusion protein form, etc., is then recovered from the culture medium. Alternatively, cells can be harvested by centrifugation, disrupted by physical or chemical means, and the resulting crude extract retained for further purification. Eukaryotic or microbial cells employed in expression of proteins can be disrupted by any convenient method, including freeze-thaw cycling, sonication, mechanical disruption, or use of cell lysing agents, or other methods, which are well know to those skilled in the art. Additionally, cells expressing a HA polypeptide product of the invention can be utilized without separating the polypeptide from the cell. In such situations, the polypeptide of the invention is optionally expressed on the cell surface and is examined thus (e.g., by having HA molecules, or fragments thereof, e.g., comprising fusion proteins or the like) on the cell surface bind antibodies, etc. Such cells are also features of the invention.

Expressed polypeptides can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems known to those skilled in the art), hydroxylapatite chromatography, and lectin chromatography. Protein refolding steps can be used, as desired, in completing configuration of the mature protein. Also, high performance liquid chromatography (HPLC) can be employed in the final purification steps.

Alternatively, cell-free transcription/translation systems can be employed to produce polypeptides comprising an amino acid sequence or subsequence of the invention. A number of suitable in vitro transcription and translation systems are commercially available. A general guide to in vitro transcription and translation protocols is found in Tymms (1995) In vitro Transcription and Translation Protocols: Methods in Molecular Biology Volume 37, Garland Publishing, NY.

In addition, the polypeptides, or subsequences thereof; e.g., subsequences comprising antigenic peptides, can be produced manually or by using an automated system, by direct peptide synthesis using solid-phase techniques (see, Merrifield J 1963 J Am Chem Soc 85:2149-2154). Exemplary automated systems include the Applied Biosystems 431 A Peptide Synthesizer (Perkin Elmer, Foster City, Calif.). If desired, subsequences can be chemically synthesized separately, and combined using chemical methods to provide full length polypeptides.

### Modified Amino Acids

Expressed polypeptides of the invention can contain one or more modified amino acids. The presence of modified amino acids can be advantageous in, for example, (a) increasing polypeptide serum half-life, (b) reducing/increasing polypeptide antigenicity, (c) increasing polypeptide storage stability, etc. Amino acid(s) are modified, for example, co-translationally or post-translationally during recombinant production (e.g., N-linked glycosylation at N—X—S/T motifs during expression in mammalian cells) or modified by synthetic means (e.g., via PEGylation).

Non-limiting examples of a modified amino acid include a glycosylated amino acid, a sulfated amino acid, a prenylated (e.g., farnesylated, geranylgeranylated) amino acid, an acetylated amino acid, an acylated amino acid, a PEG-ylated amino acid, a biotinylated amino acid, a carboxylated amino acid, a phosphorylated amino acid, and the like, as well as mono acids modified by conjugation to, e.g., lipid moieties or other organic derivatizing agents. References adequate to guide one of skill in the modification of amino acids are replete throughout the literature. Example protocols are found in Walker (1998) Protein Protocols on CD-ROM Human Press, Towata, N.J.

### Fusion Proteins

The present invention also provides fusion proteins comprising fusions of the sequences of the invention (e.g., encoding HA polypeptides) or fragments thereof with, e.g., immunoglobulins (or portions thereof), sequences encoding, e.g., GFP (green fluorescent protein), or other similar markers, etc. Nucleotide sequences encoding such fusion proteins are another aspect of the invention. Fusion proteins of the invention are optionally used for, e.g., similar applications (including, e.g., therapeutic, prophylactic, diagnostic, experimental, etc. applications as described herein) as the non-fusion proteins of the invention. In addition to fusion with immunoglobulin sequences and marker sequences, the proteins of the invention are also optionally fused with, e.g., targeting of the fusion proteins to specific cell types, regions, etc.

### Antibodies

The polypeptides of the invention can be used to produce antibodies specific for the polypeptides given herein and/or polypeptides encoded by the polynucleotides of the invention, e.g., those shown herein, and conservative variants thereof. Antibodies specific for the above mentioned polypeptides are useful, e.g., for diagnostic and therapeutic purposes, e.g., related to the activity, distribution, and expression of target polypeptides. For example, such antibodies can optionally be utilized to define other viruses within the same strain(s) as the HA sequences herein.

Antibodies specific for the polypeptides of the invention can be generated by methods well known in the art. Such antibodies can include, but are not limited to, polyclonal, monoclonal, chimeric, humanized, single chain, Fab fragments and fragments produced by an Fab expression library.

Polypeptides do not require biological activity for antibody production (e.g., full length functional hemagglutinin is not required). However, the polypeptide or oligopeptide must be antigenic. Peptides used to induce specific antibodies typically have an amino acid sequence of at least about 4 amino acids, and often at least 5 or 10 amino acids. Short stretches of a polypeptide can be fused with another protein, such as keyhole limpet hemocyanin, and antibody produced against the chimeric molecule.

Numerous methods for producing polyclonal and monoclonal antibodies are known to those of skill in the art, and can be adapted to produce antibodies specific for the polypeptides of the invention, and/or encoded by the polynucleotide sequences of the invention, etc. See, e.g., Harlow and Lane (1988) Antibodies: A Laboratory Manual Cold Spring Harbor Press, NY; and Kohler and Milstein (1975) Nature 256: 495-497. Other suitable techniques for antibody preparation include selection of libraries of recombinant antibodies in phage or similar vectors. See, Huse et al. 1989 Science 246:1275-1281; and Ward, et al. 1989 Nature 341:544-546. Specific monoclonal and polyclonal antibodies and antisera will usually bind with a KD of, e.g., at least about 0.1 at least about 0.01 μM or better, and, typically at least about 0.001 μM or better.

For certain therapeutic applications, humanized antibodies are desirable. Detailed methods for preparation of chimeric (humanized) antibodies can be found in U.S. Pat. No. 5,482,856. Additional details on humanization and other antibody production and; engineering techniques can be found in the patent and scientific literature.

### Defining Polypeptides by Immunoreactivity

Because the polypeptides of the invention provide a variety of new polypeptide sequences (e.g., comprising HA molecules), the polypeptides also provide new structural features which can be recognized, e.g., in immunological assays. The generation of antisera which specifically bind the polypeptides of the invention, as well as the polypeptides which are bound by such antisera, are features of the invention.

For example, the invention includes polypeptides (e.g., HA molecules) that specifically bind to or that are specifically immunoreactive with an antibody or antisera generated against an immunogen comprising an amino acid sequence selected from one or more of the sequences given herein, etc. To eliminate cross-reactivity with other homologues, the antibody or antisera is subtracted with the HA molecules found in public databases at the time of filing, e.g., the “control” polypeptide(s). Where the other control sequences correspond to a nucleic acid, a polypeptide encoded by the nucleic acid is generated and used for antibody/antisera subtraction purposes.

In one typical format, the immunoassay uses a polyclonal antiserum which was raised against one or more polypeptide comprising one or more of the sequences corresponding to the sequences herein, etc. or a substantial subsequence thereof (i.e., at least about 30% of the full length sequence provided). The set of potential polypeptide immunogens derived from the present sequences are collectively referred to below as “the immunogenic polypeptides”. The resulting antisera is optionally selected to have low cross reactivity against the control hemagglutinin homologues and any such cross-reactivity is removed, e.g., by immunoabsorption, with one or more of the control hemagglutinin homologues, prior to use of the polyclonal antiserum in the immunoassay.

In order to produce antisera for use in an immunoassay, one or more of the immunogenic polypeptides is produced and purified as described herein. For example, recombinant protein can be produced in a recombinant cell. An inbred strain of mice (used in this assay because results are more reproducible due to the virtual genetic identity of the mice) is immunized with the immunogenic protein(s) in combination with a standard adjuvant, such as Freund's adjuvant, and a standard mouse immunization protocol (see, e.g., Harlow and Lane (1988) Antibodies, A Laboratory Manual, Cold Spring Harbor Publications, New York, for a standard description of antibody generation, immunoassay formats and conditions that can be used to determine specific immunoreactivity). Additional references and discussion of antibodies is also found herein and can be applied here to defining polypeptides by immunoreactivity. Alternatively, one or more synthetic or recombinant polypeptides derived from the sequences disclosed herein is conjugated to a carrier protein and used as an immunogen.

Polyclonal sera are collected and titered against the immunogenic polypeptide in an immunoassay, for example, a solid phase immunoassay with one or more of the immunogenic proteins immobilized on a solid support. Polyclonal antisera with a titer of 10⁶ or greater are selected, pooled and subtracted with the control hemagglutinin polypeptide(s) to produce subtracted pooled titered polyclonal antisera.

The subtracted pooled titered polyclonal antisera are tested for cross reactivity against the control homologue(s) in a comparative immunoassay. In this comparative assay, discriminatory binding conditions are determined for the subtracted titered polyclonal antisera which result in at least about a 5-10 fold higher signal to noise ratio for binding of the titered polyclonal antisera to the immunogenic polypeptides as compared to binding to the control homologues. That is, the stringency of the binding reaction is adjusted by the addition of non-specific competitors such as albumin or non-fat dry milk, and/or by adjusting salt conditions, temperature, and/or the like. These binding conditions are used in subsequent assays for determining whether a test polypeptide (a polypeptide being compared to the immunogenic polypeptides and/or the control polypeptides) is specifically bound by the pooled subtracted polyclonal antisera. In particular, test polypeptides which show at least a 2-5× higher signal to noise ratio than the control homologues under discriminatory binding conditions, and at least about a ½ signal to noise ratio as compared to the immunogenic polypeptide(s), share substantial structural similarity with the immunogenic polypeptide as compared to the control, etc., and is, therefore a polypeptide of the invention.

In another example, immunoassays in the competitive binding format are used for detection of a test polypeptide. For example, as noted, cross-reacting antibodies are removed from the pooled antisera mixture by immunoabsorption with the control polypeptides. The immunogenic polypeptide(s) are then immobilized to a solid support which is exposed to the subtracted pooled antisera. Test proteins are added to the assay to compete for binding to the pooled subtracted antisera. The ability of the test protein(s) to compete for binding to the pooled subtracted antisera as compared to the immobilized protein(s) is compared to the ability of the immunogenic polypeptide(s) added to the assay to compete for binding (the immunogenic polypeptides compete effectively with the immobilized immunogenic polypeptides for binding to the pooled antisera). The percent cross-reactivity for the test proteins is calculated, using standard calculations.

In a parallel assay, the ability of the control protein(s) to compete for binding to the pooled subtracted antisera is optionally determined as compared to the ability of the immunogenic polypeptide(s) to compete for binding to the antisera. Again, the percent cross-reactivity for the control polypeptide(s) is calculated, using standard calculations. Where the percent cross-reactivity is at least 5-10× as high for the test polypeptides as compared to the control polypeptide(s) and or where the binding of the test polypeptides is approximately in the range of the binding of the immunogenic polypeptides, the test polypeptides are said to specifically bind the pooled subtracted antisera.

In general, the immunoabsorbed and pooled antisera can be used in a competitive binding immunoassay as described herein to compare any test polypeptide to the immunogenic and/or control polypeptide(s). In order to make this comparison, the immunogenic, test and control polypeptides are each assayed at a wide range of concentrations and the amount of each polypeptide required to inhibit 50% of the binding of the subtracted antisera to, e.g., an immobilized control, test or immunogenic protein is determined using standard techniques. If the amount of the test polypeptide required for binding in the competitive assay is less than twice the amount of the immunogenic polypeptide that is required, then the test polypeptide is said to specifically bind to an antibody generated to the immunogenic protein, provided the amount is at least about 5-10× as high as for the control polypeptide.

As an additional determination of specificity, the pooled antisera is optionally fully immunosorbed with the immunogenic polypeptide(s) (rather than the control polypeptide(s)) until little or no binding of the resulting immunogenic polypeptide subtracted pooled antisera to the immunogenic polypeptide(s) used in the immunosorbtion is detectable. This fully immunosorbed antisera is then tested for reactivity with the test polypeptide. If little or no reactivity is observed (i.e., no more than 2× the signal to noise ratio observed for binding of the fully immunosorbed antisera to the immunogenic polypeptide), then the test polypeptide is specifically bound by the antisera elicited by the immunogenic protein.

### Nucleic Acid and Polypeptide Sequence Variants

As described herein, the invention provides for nucleic acid polynucleotide sequences and polypeptide amino acid sequences, e.g., hemagglutinin sequences, and, e.g., compositions and methods comprising said sequences. Examples of said sequences are disclosed herein. However, one of skill in the art will appreciate that the invention is not necessarily limited to those sequences-disclosed herein and that the present invention also provides many related and unrelated sequences with the functions described herein, e.g., encoding a HA molecule.

One of skill will also appreciate that many variants of the disclosed sequences are included in the invention. For example, conservative variations of the disclosed sequences that yield a functionally identical sequence are included in the invention. Variants of the nucleic acid polynucleotide sequences, wherein the variants hybridize to at least one disclosed sequence, are considered to be included in the invention. Subsequences of the sequences disclosed herein are also included in the invention.

### Silent Variations

Due to the degeneracy of the genetic code, any of a variety of nucleic acid sequences encoding polypeptides of the invention are optionally produced, some which can bear lower levels of sequence identity to the HA nucleic acid and polypeptide sequences herein. Codon tables specifying the genetic code are found in many biology and biochemistry texts. Such codon tables show that many amino acids are encoded by more than one codon. For example, the codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that U in an RNA sequence corresponds to T in a DNA sequence.

Such “silent variations” are one species of “conservatively modified variations,” discussed below. One of skill will recognize that each codon in a nucleic acid (except ATG, which is ordinarily the only codon for methionine, and TTG, which is ordinarily the only codon for tryptophan) can be modified by standard techniques to encode a functionally identical polypeptide. Accordingly, each silent variation of a nucleic acid which encodes a polypeptide is implicit in any described sequence. The invention, therefore, explicitly provides each and every possible variation of a nucleic acid sequence encoding a polypeptide of the invention that could be made by selecting combinations based on possible codon choices, including human-preferred codons. These combinations are made in accordance with the standard, triplet genetic code as applied to the nucleic acid sequence encoding a hemagglutinin polypeptide of the invention. All such variations of every nucleic acid herein are specifically provided and described by consideration of the sequence in combination with the genetic code. One of skill is fully able to make these silent substitutions using the methods herein.

Conservative variations

Owing to the degeneracy of the genetic code, “silent substitutions” (i.e., substitutions in a nucleic acid sequence which do not result in an alteration in an encoded polypeptide) are an implied feature of every nucleic acid sequence of the invention which encodes an amino acid. Similarly, “conservative amino acid substitutions,” in one or a few amino acids in an amino acid sequence are substituted with different amino acids with highly similar properties, are also readily identified as being highly similar to a disclosed construct such as those herein. Such conservative variations of each disclosed sequence are a feature of the present invention.

“Conservative variation” of a particular nucleic acid sequence refers to those nucleic acids which encode identical or essentially identical amino acid sequences, or, where the nucleic acid does not encode an amino acid sequence, to essentially identical sequences, see, Table 3 below. One of skill will recognize that individual substitutions, deletions or additions which alter, add or delete a single amino acid or a small percentage of amino acids (typically less than 5%, more typically less than 4%, 3%, 2% or 1%) in an encoded sequence are “conservatively modified variations” where the alterations result in the deletion of an amino acid, addition of an amino acid, or substitution of an amino acid with a chemically similar amino acid. Thus, “conservative variations” of a listed polypeptide sequence of the present invention include substitutions of a small percentage, typically less than 5%, more typically less than 4%, 3%, 2% or 1%, of the amino acids of the polypeptide sequence, with a conservatively selected amino acid of the same conservative substitution group. Finally, the addition of sequences which do not alter the encoded activity of a nucleic acid molecule, such as the addition of a non-functional sequence, is a conservative variation of the basic nucleic acid.

TABLE 3
Conservative Substitution Groups

| Group | Amino Acids |
|---|---|
| 1 | Alanine (A), Serine(S), Threonine (T) |
| 2 | Aspartic acid (D) Glutamic acid (E) |
| 3 | Asparagine (N) Glutamine (Q) |
| 4 | Arginine (R) Lysine (K) |
| 5 | Isoleucine (I) Leucine (L), Methionine (M) Valine (V) |
| 6 | Phenylalanine (F) Tyrosine (Y) Tryptophan (W) |

### Sequence Comparison, Identity, and Homology

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection.

The phrase “substantially identical,” in the context of two nucleic acids or polypeptides (e.g., DNAs encoding an HA molecule, or the amino acid sequence of an HA molecule) refers to two or more sequences or subsequences that have at least about 90%, preferably 91%, most preferably 92%, 93%, 94%, 95%, 96%, 97%, 98%, 98.5%, 99%, 99.1%, 99.2%, 99.3%, 99.4%, 99.5%, 99.6%, 99.7%, 99.8%, 99.9% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, “substantial identity” exists over a region of the amino acid sequences that is at least about 200 residues in length, more preferably over a region of at least about 250 residues, and most preferably the sequences are substantially identical over at least about 300 residues, 350 residues, 400 residues, 425 residues, 450 residues, 475 residues, 480 residues, 490 residues, 495 residues, 499 residues, or 500 residues, or over the full length of the two sequences to be compared when the amino acids are hemagglutinin or hemagglutinin fragments.

For sequence comparison and homology determination, typically one sequence acts as a reference sequence to which test sequences are compared. When using a sequence comparison algorithm, test and reference sequences are input into a computer, subsequence coordinates are designated, if necessary; and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, Proc Natl Acad Sci USA 85:2444 (1988), by computerized implementations of algorithms such as GAP, BESTFIT, FASTA, and TFASTA in the Wisconsin Genetics Software Package, Genetics Computer Group, 575 Science Dr., Madison, Wis., or by visual inspection.

One example of an algorithm that is suitable for determining percent sequence identity and sequence similarity is the BLAST algorithm, which is described in Altschul et al., J Mol Biol 215:403-410 (1990). Software for performing BLAST analyses is publicly available through the National Center for Biotechnology Information, on the world-wide-web at ncbi.nlm.nih.gov. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see, Henikoff & Henikoff (1989) Proc Natl Acad Sci USA 89:10915).

In addition to calculating percent sequence identity, the BLAST algorithm also performs a statistical analysis of the similarity between two sequences (see, e.g., Karlin & Altschul, Proc Natl Acad Sci USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another example of a useful sequence alignment algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5:151-153. The program can align, e.g., up to 300 sequences of a maximum length of 5,000 letters. The multiple alignment procedure begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. The final alignment is achieved by a series of progressive, pairwise alignments. The program can also be used to plot a dendogram or tree representation of clustering relationships. The program is run by designating specific sequences and their amino acid or nucleotide coordinates for regions of sequence comparison.

An additional example of an algorithm that is suitable for multiple DNA, or amino acid, sequence alignments is the CLUSTALW program (Thompson, J. D. et al. (1994) Nucl Acids Res 22: 4673-4680). CLUSTALW performs multiple pairwise comparisons between groups of sequences and assembles them into a multiple alignment based on homology. Gap open and Gap extension penalties can be, e.g., 10 and 0.05 respectively. For amino acid alignments, the BLOSUM algorithm can be used as a protein weight matrix. See, e.g., Henikoff and Henikoff (1992) Proc. Natl. Acad. Sci. USA 89: 10915-10919.

### Methods and Compositions for Prophylactic Administration of Vaccines

In general, the embodiments of the current invention can be administered prophylactically in an immunologically effective amount and in an appropriate carrier or excipient to stimulate an immune response specific for one or more strains of influenza virus as determined by the HA sequence. Typically, the carrier or excipient is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions insuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, etc.

A related aspect of the invention provides methods for stimulating the immune system of an individual to produce a protective immune response against influenza virus. In the methods, an immunologically effective amount of the embodiments of the present invention (e.g., an HA molecule of the invention), an immunologically effective amount of a polypeptide of the invention, and/or an immunologically effective amount of a nucleic acid of the invention is administered to the individual in a physiologically acceptable carrier.

Generally, the embodiments of the invention are administered in a quantity sufficient to stimulate an immune response specific for one or more strains of influenza virus (i.e., against the HA strains of the invention). Preferably, administration of the embodiments of the invention elicits a protective immune response to such strains. Dosages and methods for eliciting a protective immune response against one or more influenza strains are known to those of skill in the art. Typically, the dose will be adjusted within a range based on, e.g., age, physical condition, body weight, sex, diet, time of administration, and other clinical factors. The prophylactic vaccine formulation is systemically administered, e.g., by subcutaneous or intramuscular injection using a needle and syringe, or a needle-less injection device. Alternatively, the vaccine formulation is administered intranasally, either by drops, large particle aerosol (greater than about 10 microns), or spray into the upper respiratory tract. While any of the above routes of delivery results in a protective systemic immune response, intranasal administration confers the added benefit of eliciting mucosal immunity at the site of entry of the influenza virus. While stimulation of a protective immune response with a single dose is preferred, additional dosages can be administered, by the same or different route, to achieve the desired prophylactic effect.

In neonates and infants, for example, multiple administrations may be required to elicit sufficient levels of immunity. Administration can continue at intervals throughout childhood, as necessary to maintain sufficient levels of protection against wild-type influenza infection. Similarly, adults who are particularly susceptible to repeated or serious influenza infection, such as, for example, health care workers, day care workers, family members of young children, the elderly, and individuals with compromised cardiopulmonary function may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

Optionally, the formulation for prophylactic administration of the embodiments of the invention also contains one or more adjuvants for enhancing the immune response to the influenza antigens. Suitable adjuvants include: complete Freund's adjuvant, incomplete Freund's adjuvant, saponin, mineral gels such as aluminum hydroxide, and surface active substances such as lysolecithin, pluronic polyols, polyanions, peptides, oil or hydrocarbon emulsions, bacille Calmette-Guerin (BCG), Corynebacterium parvam, and the synthetic adjuvants QS-21 and MF59.

If desired, prophylactic vaccine administration of embodiments of the invention can be performed in conjunction with administration of one or more immunostimulatory molecules. Immunostimulatory molecules include various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immunostimulatory molecules can be administered in the same formulation as the embodiments of the invention, or can be administered separately. Either the protein (e.g., an HA polypeptide of the invention) or an expression vector encoding the protein can be administered to produce an immunostimulatory effect.

The above described methods are useful for therapeutically and/or prophylactically treating a disease or disorder, typically influenza, by introducing a vector of the invention comprising a heterologous polynucleotide encoding a therapeutically or prophylactically effective HA polypeptide (or peptide) or HA RNA (e.g., an antisense RNA or ribozyme) into a population of target cells in vitro, ex vivo or in vivo. Typically, the polynucleotide encoding the polypeptide (or peptide), or RNA, of interest is operably linked to appropriate regulatory sequences, e.g., as described herein. Optionally, more than one heterologous coding sequence is incorporated into a single vector or virus. For example, in addition to a polynucleotide encoding a therapeutically or prophylactically active HA polypeptide or RNA, the vector can also include additional therapeutic or prophylactic polypeptides, e.g., antigens, co-stimulatory molecules, cytokines, antibodies, etc., and/or markers, and the like.

Mutations that can Convert Avian H5 HA to Human Receptor Specificity

Avian viruses bind to sialosides with an α2-3 linkage in the intestinal tract, whereas human-adapted viruses are specific for the α2-6 linkage in the respiratory tract. A switch from α2-3 to α2-6 receptor specificity is a critical strep in the adaptation of avian viruses to a human host and appears to be one of the reasons why most avian influenza viruses, including current avian H5 strains, are not easily transmitted from human to human after avian-to-human infection.

The binding site of the receptor binding domain comprises three structural elements, namely, an a-helix (190-helix, HA1 190 to 197) and two loops (130-loop, HA1 135-138, and 220-loop, HA1 221-228). A number of conserved residues are involved in receptor binding, including amino acid positions 136, 190, 193, 194, 216, 221, 222, 225, 226, 227 and 228. Thus, the question arises as to how a current H5 virus could adapt its HA for binding to human receptors.

Previous studies have identified a number of key receptor binding domain mutations that are implicated in avian to human specificity switching in H1, H2 and H3 serotypes. For example, it was found that the 1918H1 could be converted from α2-6 receptor specificity to classic avian α2-3 specificity by only two mutations (D190E and D225G). Conversely, an avian H1 virus with α2-3 specificity was converted to α2-6 specificity by E190D and G225D mutations (Stevens J et al. 2006 Science 312:404-410). However, which mutations are likely to modulate receptor specificity in the H5 serotype is not so obvious.

In the present study, we examined the binding and entry requirements of an H5 virus by generating a series of mutants in and around the receptor binding domain to explore whether the H5 HA could readily become adapted to humans through mutations that are known to change receptor specificity in the H1 serotype. We identified amino acid differences within the HA molecule at positions that are implicated in receptor specificity. Structural and genetic differences between H1 and H5 serotypes were analyzed since they appear more closely related to one another structurally than to H3 HA. We conclude that mutations that cause a shift from the avian-type to human-type specificity on the H1 framework can also cause a shift in specificity on the H5 avian framework, permitting entry into human cells. With reference to Table 4, an embodiment of the invention is an H5 avian influenza framework comprising at least one mutation selected from the group consisting of S136T, E190D, E190N, E190G, K/R193S, K/R193A, K/R193T, K/R193N, L1941, L194F, R216E, S221P, K222W, G225D, G225N, Q226R, Q226L, S227A, S227H, S227P, S227E, S227N, and G228S. Thus, such mutations provide one possible route by which H5 viruses could gain a foothold in the human population.

TABLE 4
Conserved residues within the Receptor binding domains of H1 and H5 serotypes
that are implicated in receptor specificity.

| | Amino Acid Position | | | | | | | | | | |
|---|---|---|---|---|---|---|---|---|---|---|---|
| Serotype | 136 | 190 | 193 | 194 | 216 | 221 | 222 | 225 | 226 | 227 | 228 |
| Avian H5 (e.g., A/Thailand/1 (KAN-1) | S | E^{a} | K/R^{b} | L | R | S | K | G | Q | S | G |
| Human H1 | T | DNG | SATN | IF | E | P | W | DN | RL | AHPEN | S |

^{a}Exception, A/Vietnam/CL01/2004, position 190 is D.
^{b}Exception, A/Dk/HN/303/2004, position 193 is S.

### Triple-mutant HA

Influenza virus entry is mediated by its spike glycoprotein, the viral hemagglutinin (HA), which is also the target of protective neutralizing antibodies elicited by preventive vaccines. The H5N1 avian influenza virus enters cells after engaging a cellular receptor, sialic acid (SA), which displays an α-2,3 linkage to galactose in avian hosts. In contrast, human-adapted viruses preferentially utilize SA with α-2,6 linkages, increasing infection of cells in the upper respiratory tract that facilitates human transmission. Here, we define mutations in the avian H5N1 HA that increase its affinity for human receptors and show that these changes alter its sensitivity to neutralizing antibodies. Structural and molecular genetic information allowed the identification of sites in the receptor binding domain that enhanced entry into human cells more than 100-fold, and lectin inhibition revealed a switch in receptor specificity. Limited to three point mutations in the receptor binding domain, the human-preferred HA was ˜10-fold more resistant to anti-H5 neutralizing antibody. These mutations rendered the HA insensitive to a neutralizing H5 monoclonal antibody; however, an alternative monoclonal antibody was identified that could neutralize both. Adaptation of H5 HA to human receptor usage therefore alters antibody sensitivity at the same time it changes receptor specificity. These findings suggest that adaptive mutations of the avian influenza virus might render current vaccines less effective. Such modified HAs nonetheless provide immunogens for therapeutic antibodies and for novel preventive vaccines that are envisioned as being developed prior to the emergence of natural human-adapted H5N1 strains.

### Immunization b Avian H5 Influenza Hemagglutinin Mutants with Altered Receptor Binding Specificity

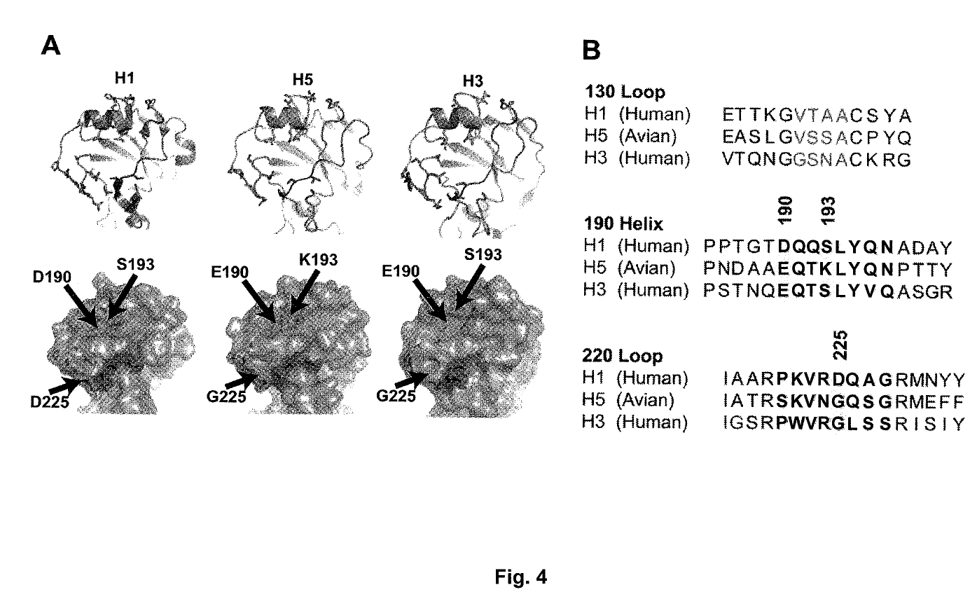

The receptor binding domain (RBD) within HA is composed of less than 300 amino acids, situated at the outer surface on top of the viral spike (Gamblin, S. J. et al. 2004 Science 303:1838; Skehel, J. J. and Wiley, D.C. 2000 Annu Rev Biochem 69:531; Stevens, J. et al. 2004 Science 303:1866; Stevens, J. et al. 2006 Science 312:404; Wilson, I. A. et al. 1981 Nature 289:366). SA binding is mediated by a cavity bordered by two ridges (FIG. 4A), formed by loop 220 (amino acids 221 to 228), loop 130 (amino acids 135 to 138), and a helical domain at amino acids 190 to 197 (numbering based on H3 A/Aichi/2/68) (Wilson, I. A. et al. 1981 Nature 289:366). The structures of the H1, H5, and H3 HAs have been previously described (Gambling, S. J. et al. 2004 Science 303:1838; Shekel, J. J. and Wiley, D. C. 2000 Annu Rev Biochem 69:531; Stevens, J. et al. 2004 Science 303:1866; Stevens, J. et al. 2006 Science 312:404; Wilson, I. A. et al. 1981 Nature 289:366), and the H1 and H5 RBD show greater structural and genetic similarity to one another than to H3 (FIG. 4A).

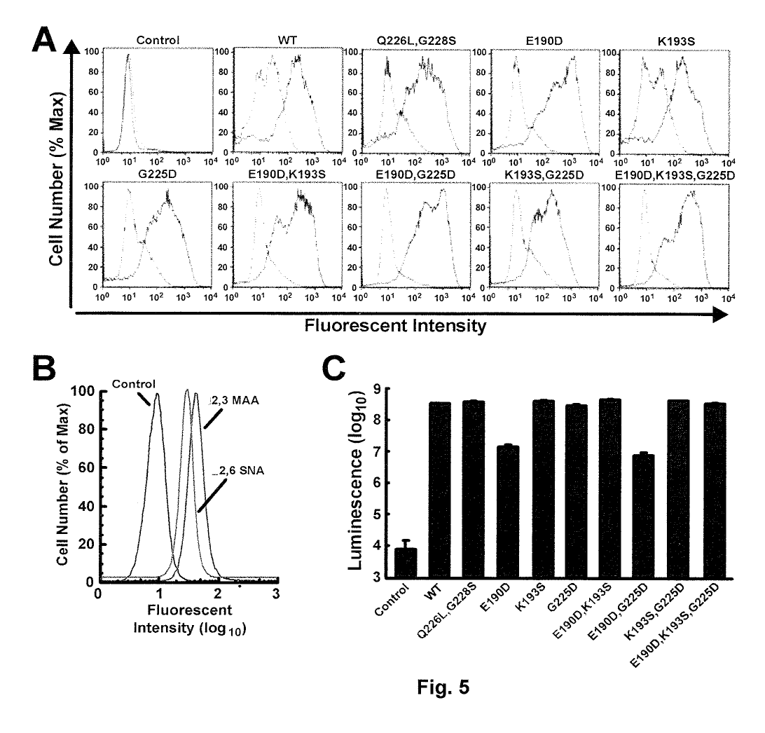

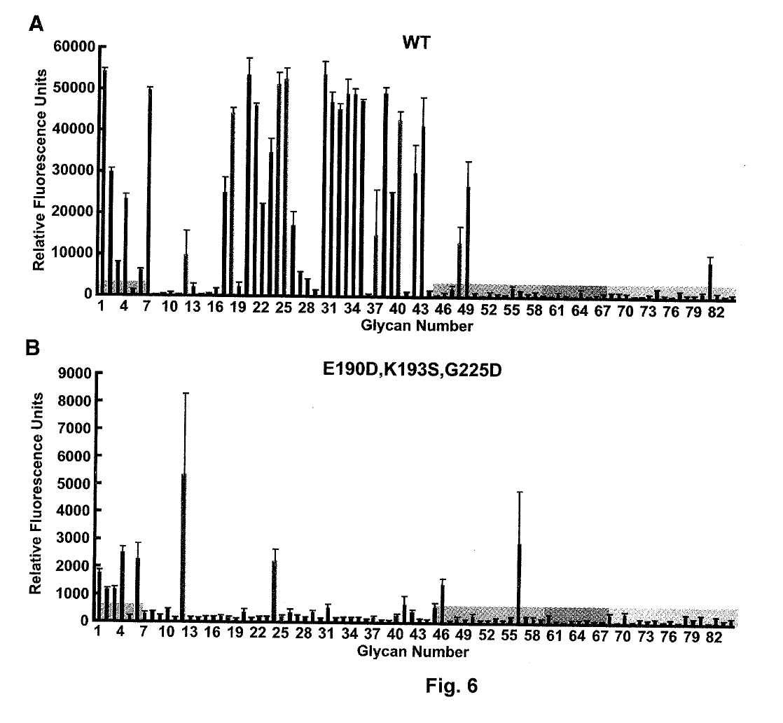

To define mutations that change receptor recognition, we focused initially on differences between H5 and H1 (A/South Carolina/1/18), which recognizes α2,6-SA linkages, particularly amino acids 190, 193, and 225 (FIG. 4B). Individual or combination mutations to create pseudoviruses were made in which amino acids were replaced at certain positions, described by the single-letter code for the amino acid, as for example, aspartic acid substituted for glutamic acid at position 190 (E190D). We also used a mutant suggested previously to increase α2,6 recognition, Q226L, G228S (Stevens, J. et al. 2006 Science 312:404). Surface expression of these HAs was confirmed by flow cytometry (FIG. 5A), and pseudotyped lentiviral vectors were produced after cotransfection of neuraminidase (NA). Entry into 293A renal epithelial cells, which express both α2,3- and α2,6-SAs (FIG. 5B), was measured with a luciferase reporter. The E190D, K193S, G225D triple-mutant virus showed entry similar to the wild-type HA (FIG. 5C), confirming its functional integrity; however, receptor specificity could not be defined with this assay.

The SA specificity of different HAs was analyzed by a modification of the glycan microarray method (Stevens, J. Et al. 2006 Nat Rev Microbiol 4:857) and by the resialylated HA assay (Paulson, J. C. and Rogers, G. N. 1987 Methods Enzymol 138:162). For glycan arrays, HAs were coexpressed with NA and purified (Stevens, J. et al. 2004 Science 303:1866). The E190D, K193S, G225D mutation eliminated recognition of most α2,3-linked substrates compared with wild-type protein (FIG. 6, A versus B). The resialylated HA assay confirmed the loss of α2,3-SA recognition in the triple mutant and lack of α2,6 binding (Table 5A), also seen in Q226L, G228S. Analysis of previously described mutants (Yamada, S. et al. 2006 Nature 444:378) also revealed no α2,6-SA recognition (Table 5B). Finally, we identified mutations that increased α2,6-SA recognition (Table 5C), particularly the S137A, T192I variant that alters both the 130 loop and 190 helix. This altered specificity was confirmed in glycan microarrays (Table 6). These mutations represent alternatives by which the HA can adapt its substrate recognition; in the last-mentioned instance, it increases 2,6-SA binding to be more similar, although not identical, to human-adapted influenza viruses.

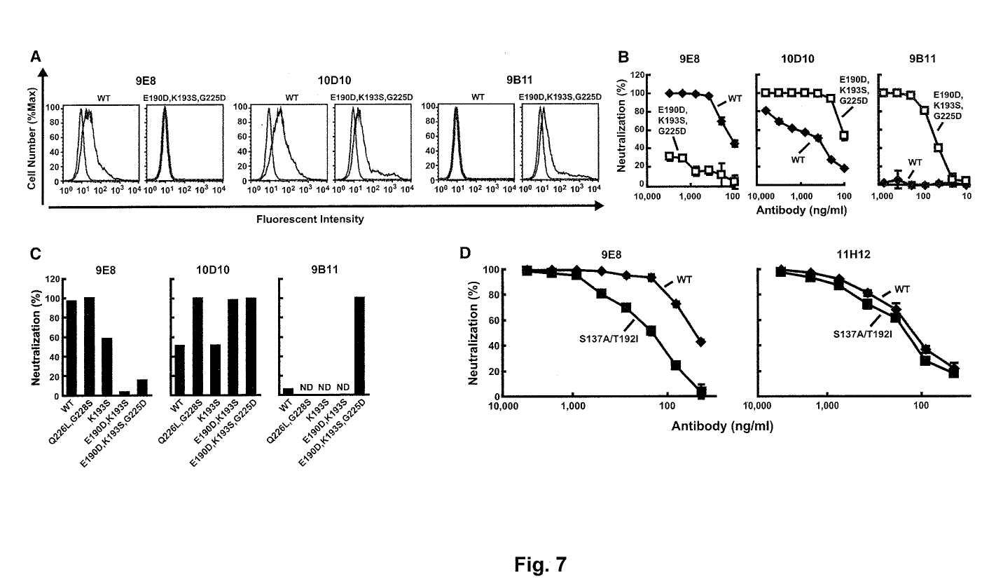

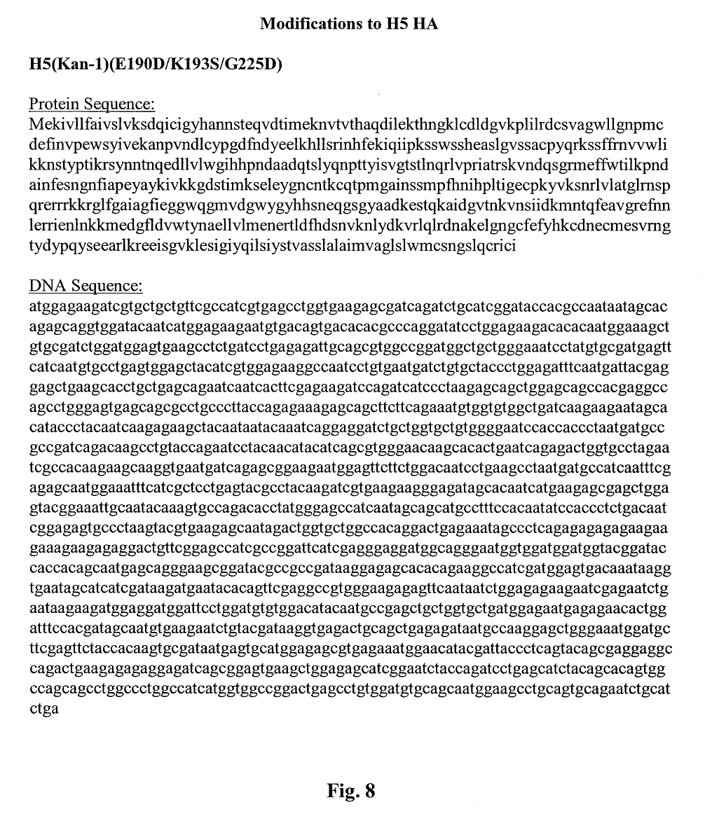

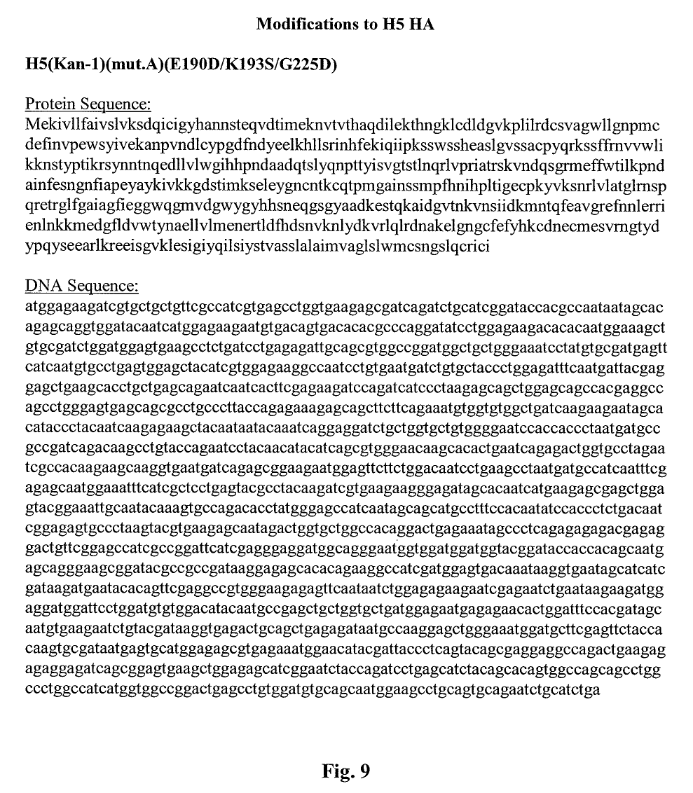

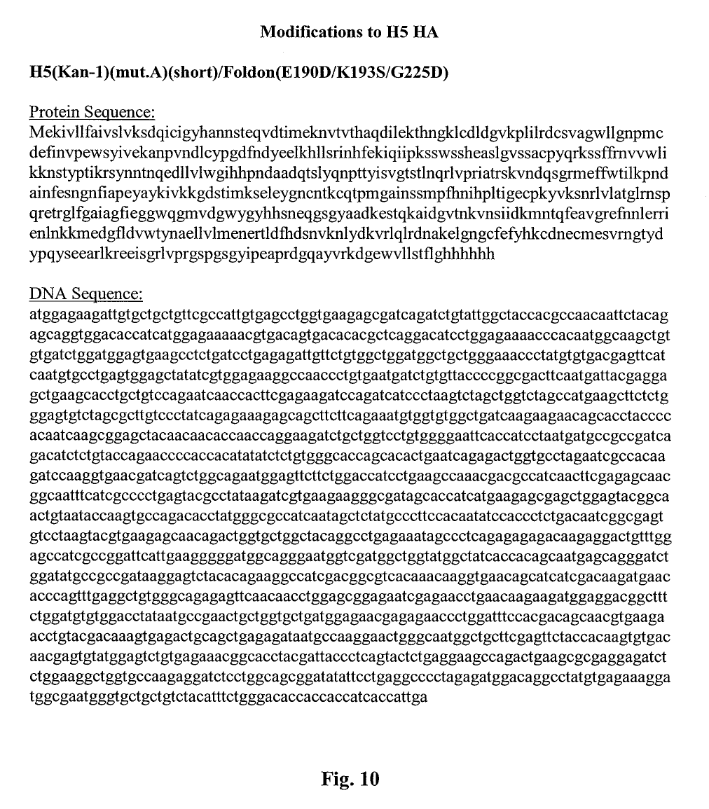

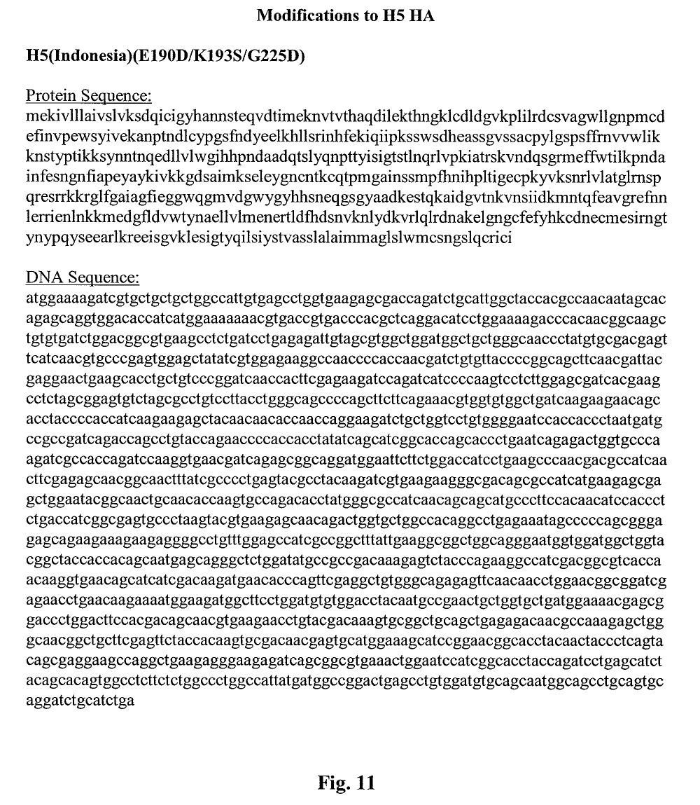

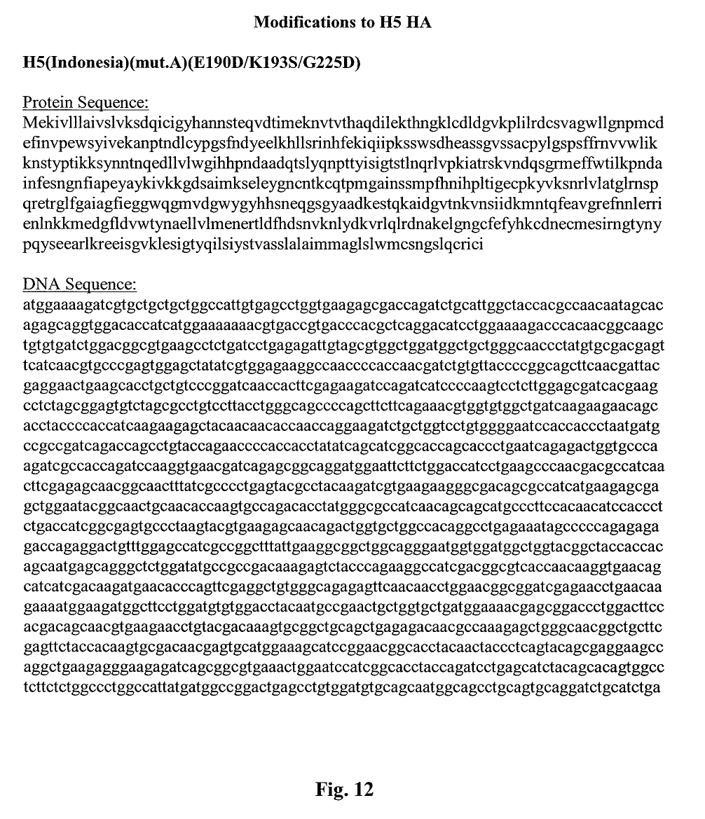

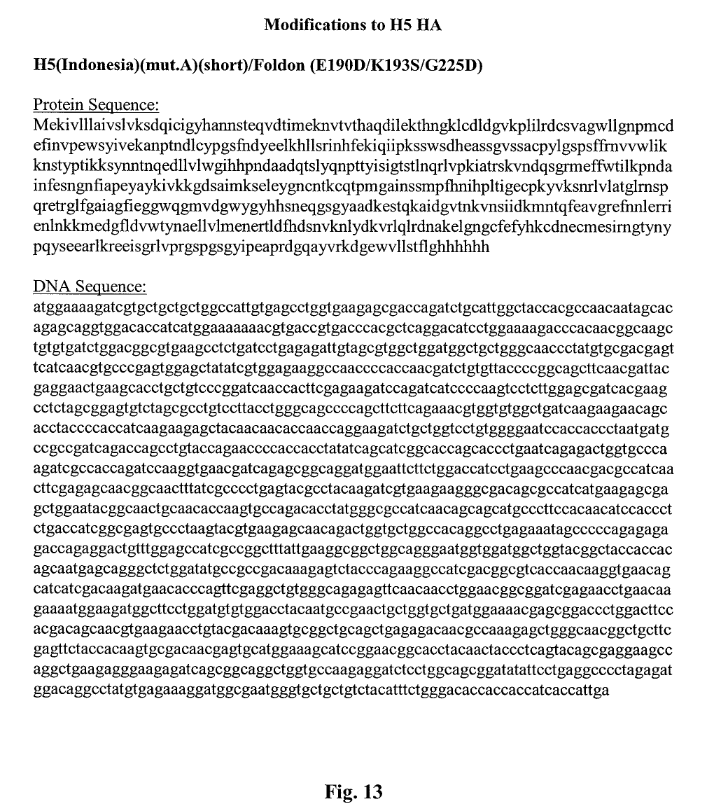

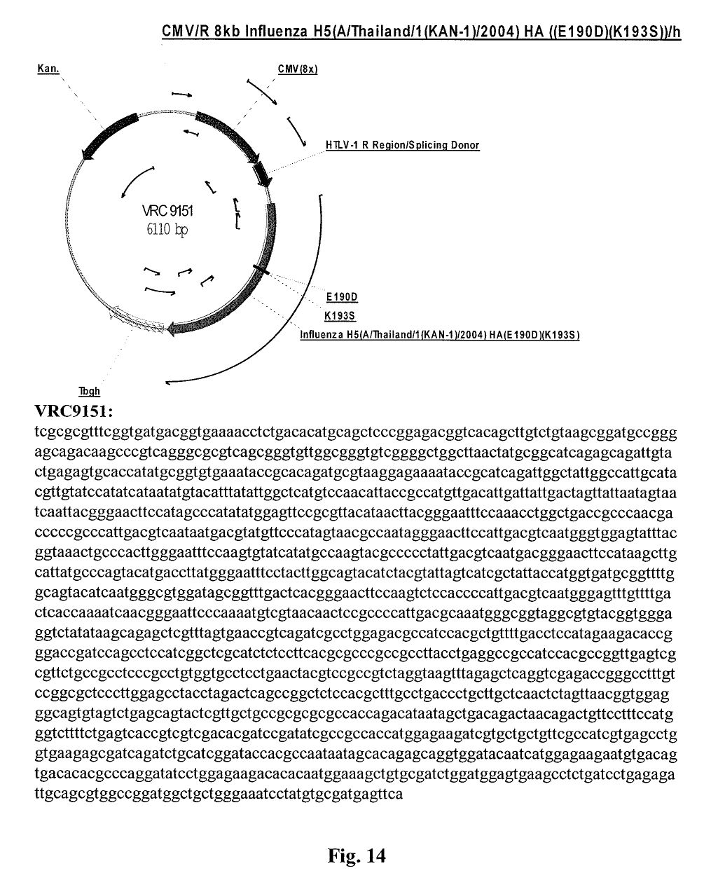

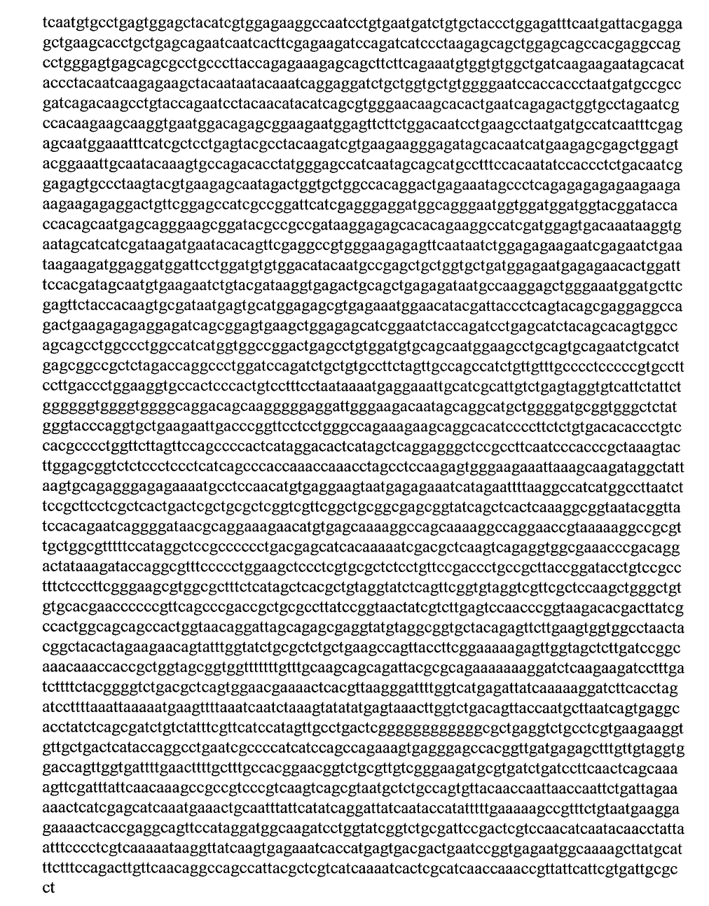

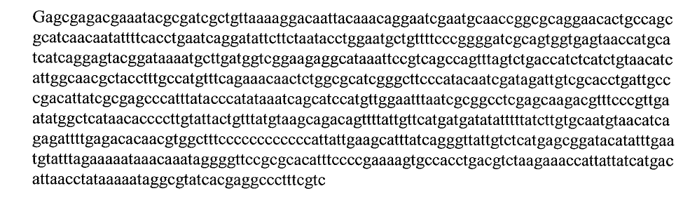

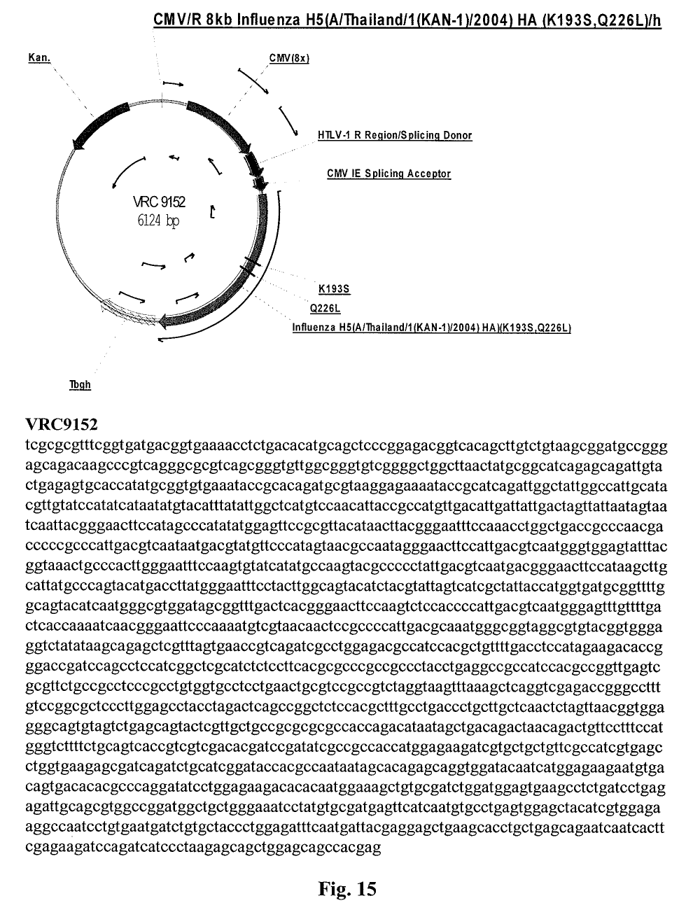

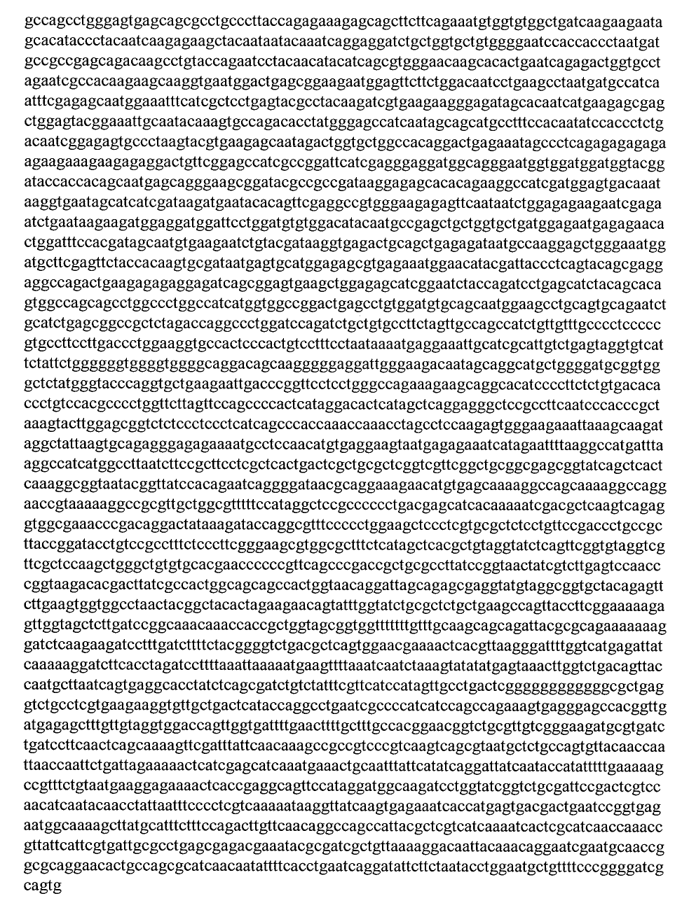

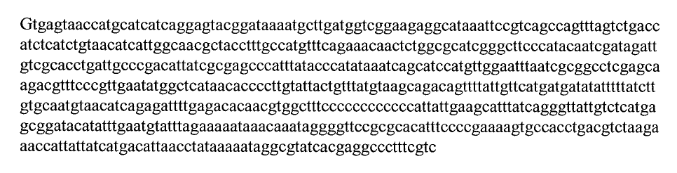

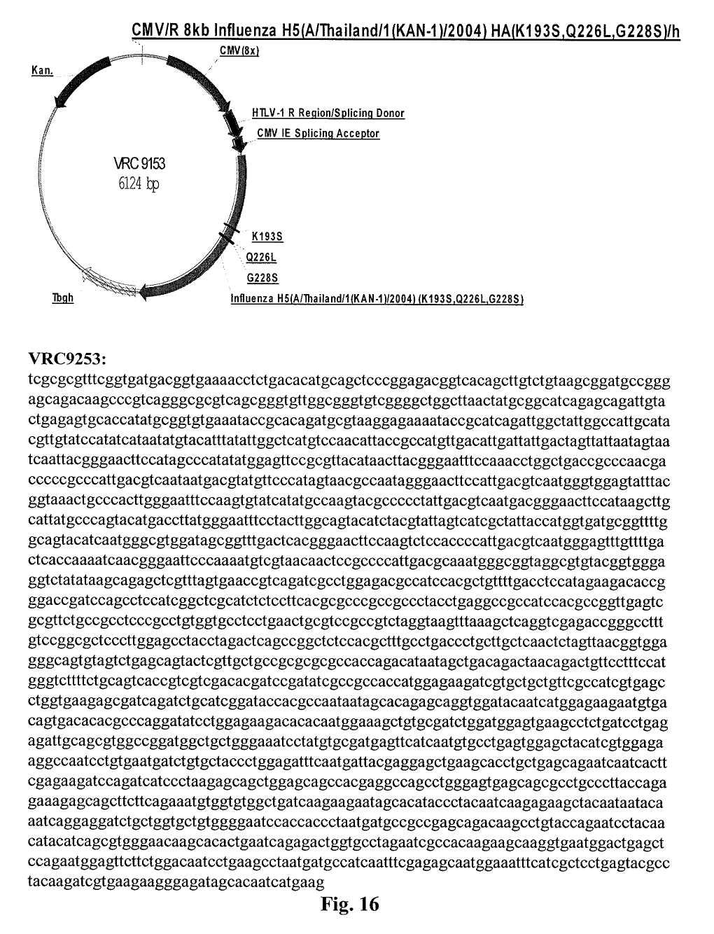

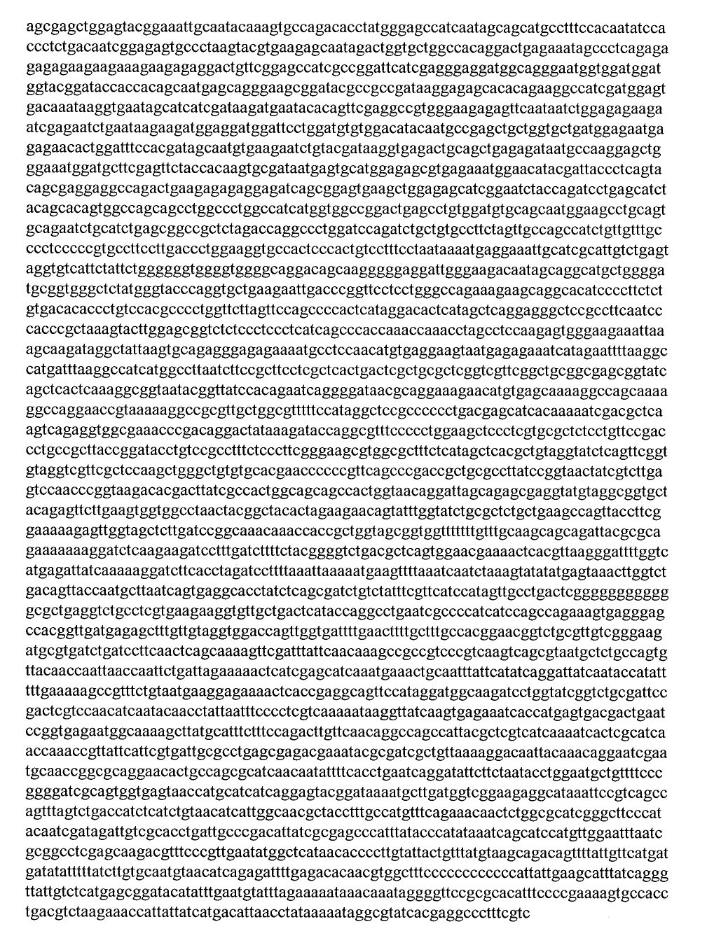

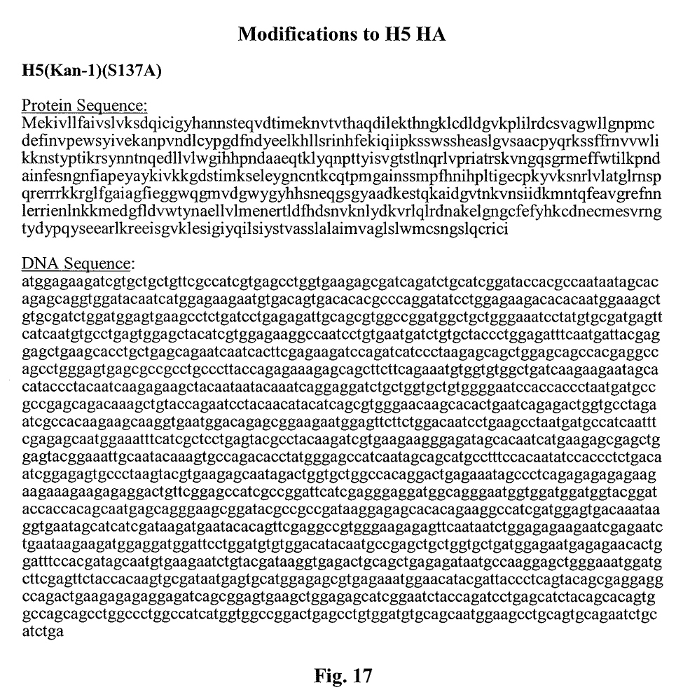

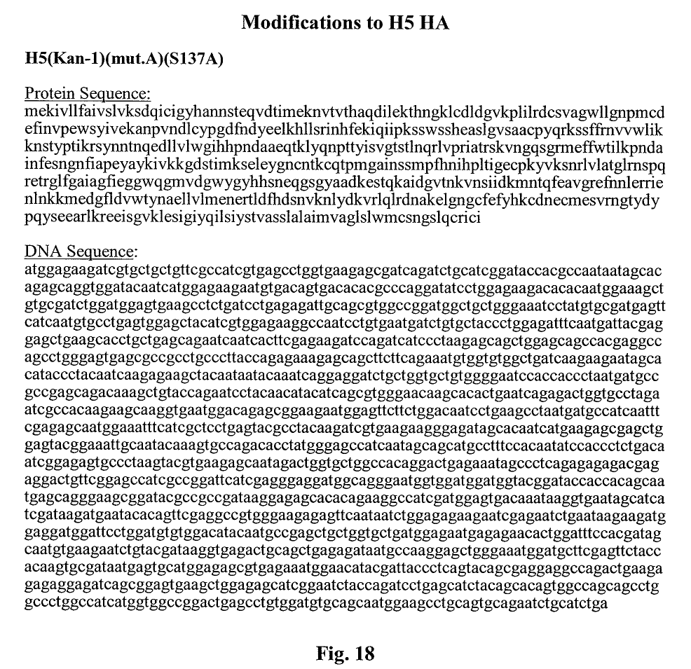

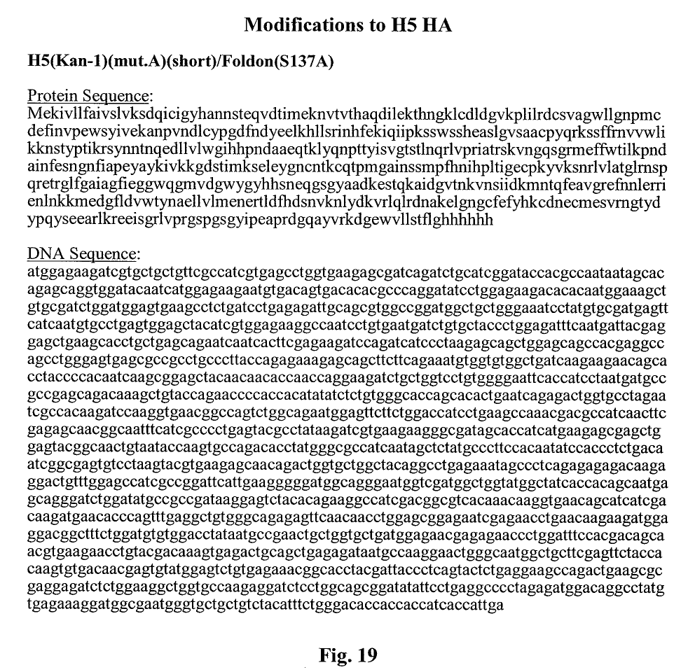

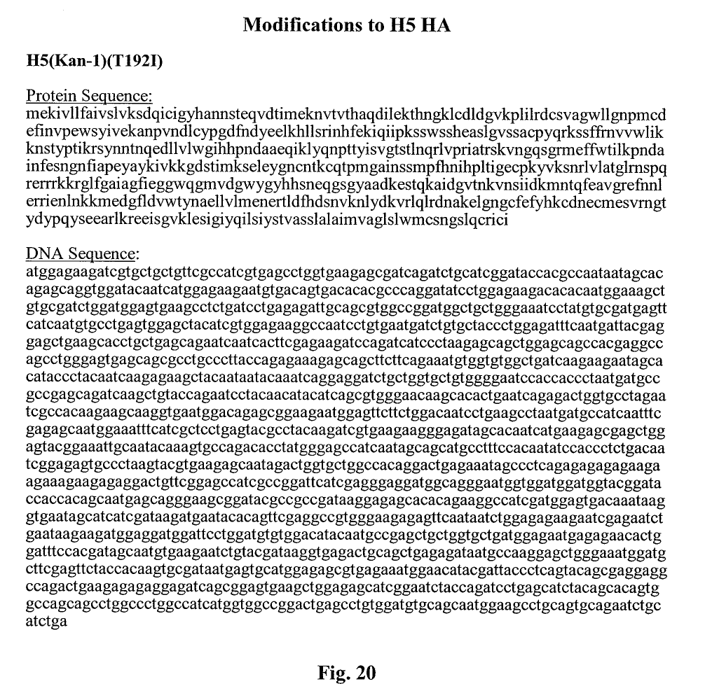

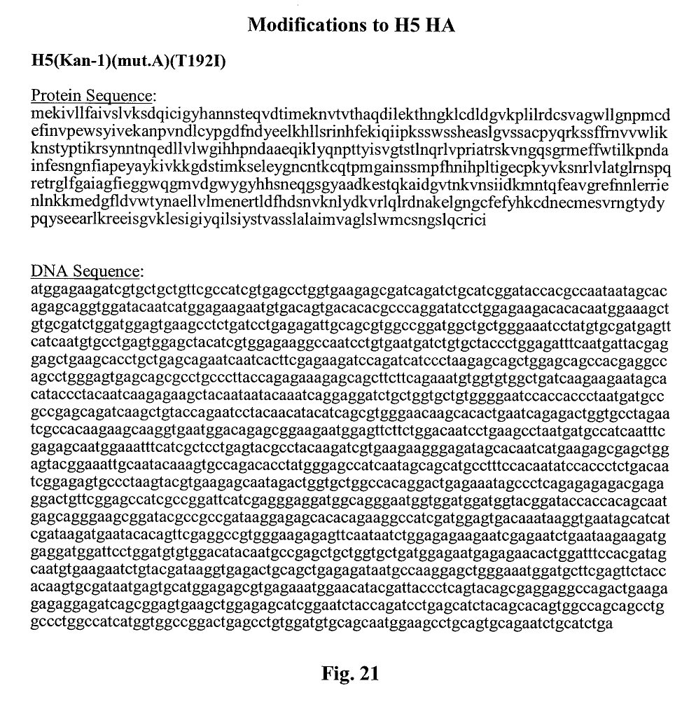

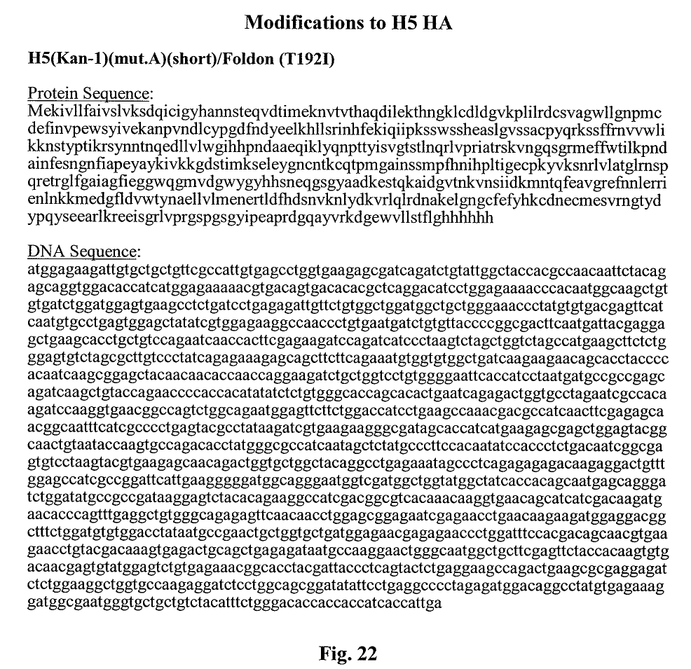

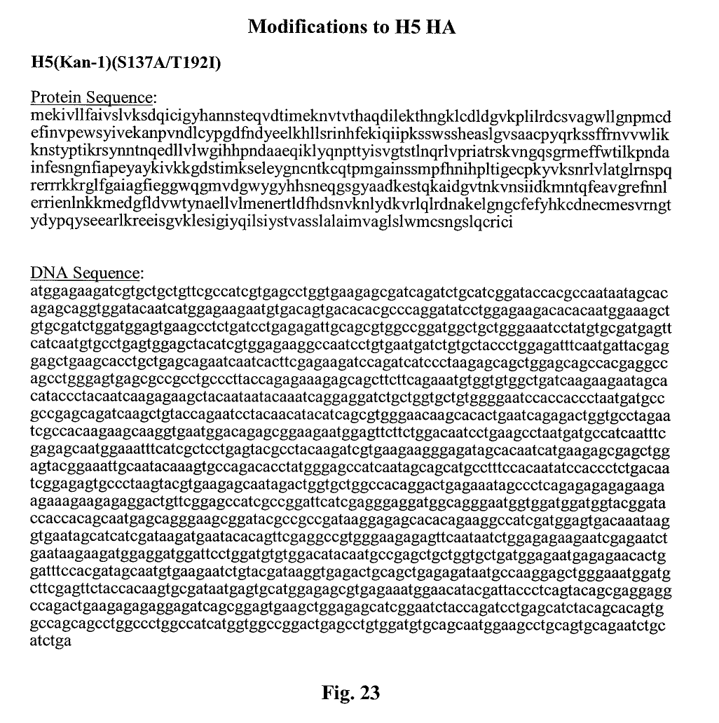

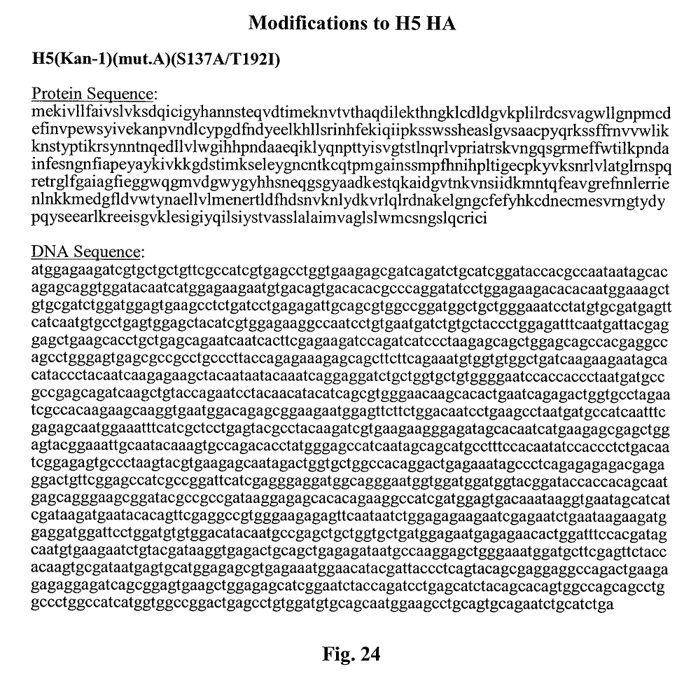

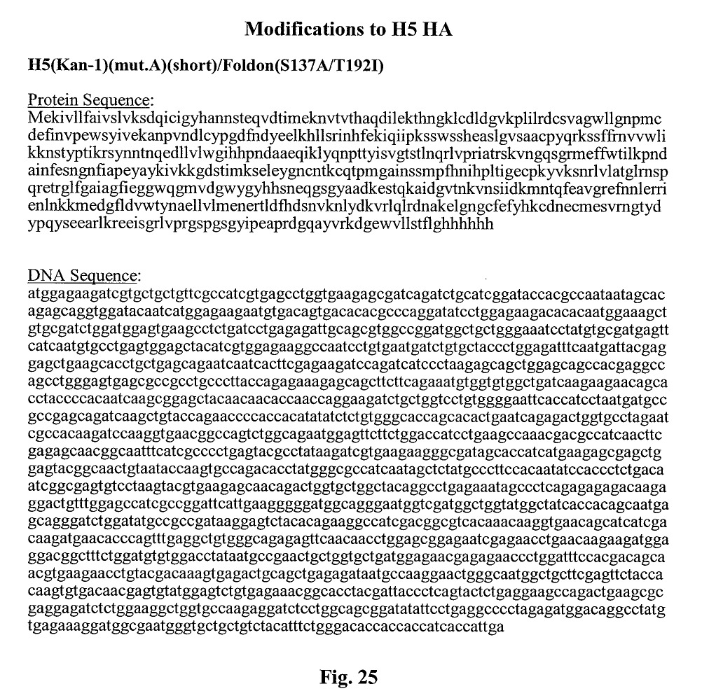

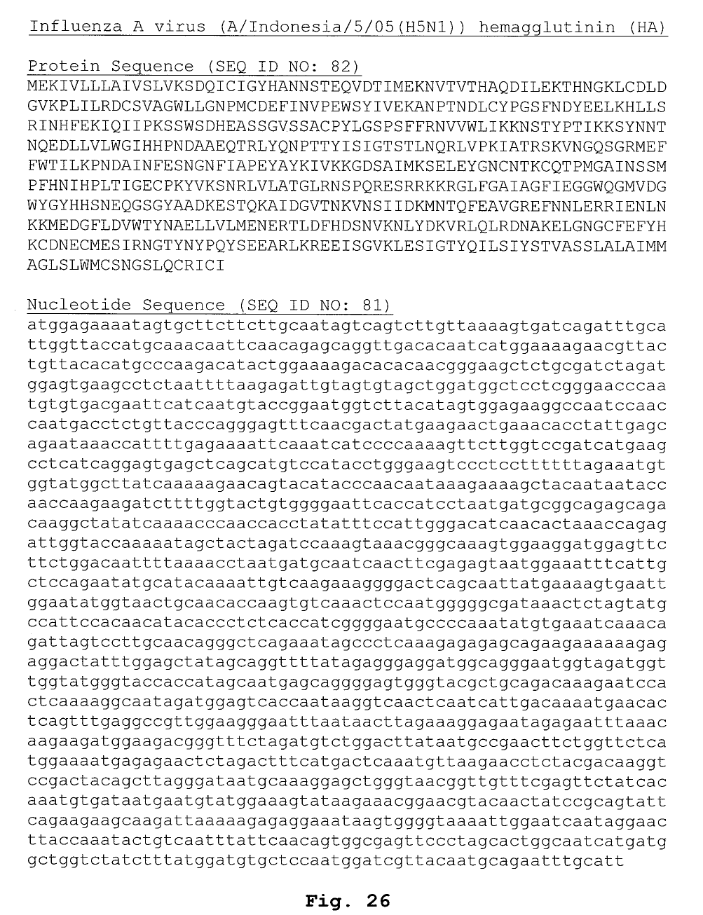

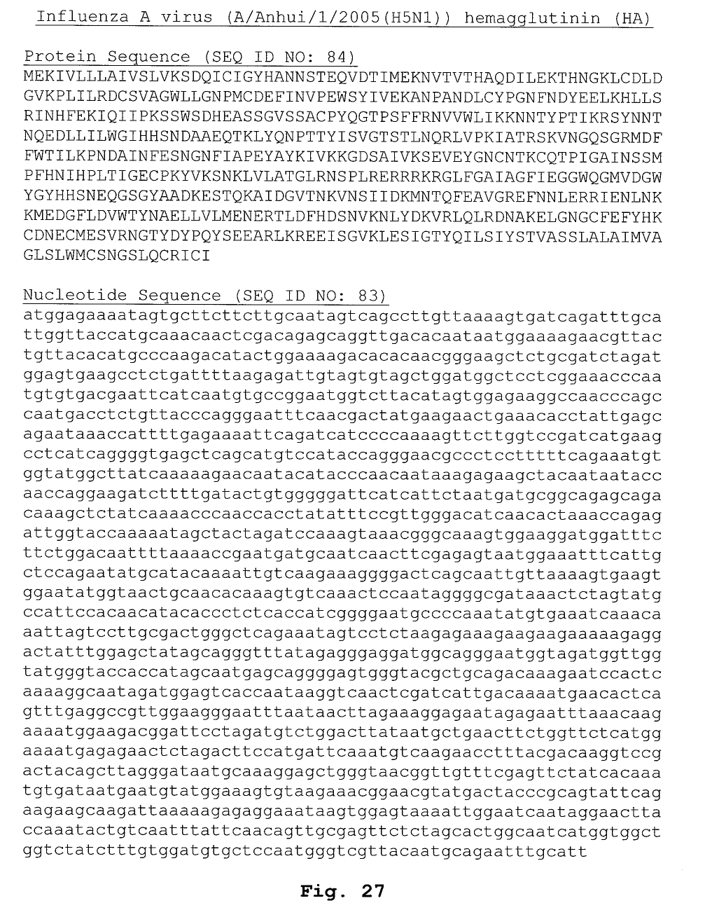

Immunogenic and antigenic differences among HAs with altered receptor specificity were analyzed by vaccination of mice with wild-type or the triple-mutant HA and generation of monoclonal antibodies (mAbs). Each mAb recognized mutant or wild-type HA coexpressed with NA with differential specificity (FIG. 7A). One potent H5-specific mAb, 9E8, neutralized wild-type H5 but showed significantly reduced activity against the triple-mutant pseudovirus (FIG. 7B, left). In contrast, a second such monoclonal, 10D10, neutralized both HAs equivalently at maximal inhibitory concentrations, although smaller differences were observed at intermediate concentrations (FIG. 7B, middle). A third mAb, 9B11, isolated after immunization with the triple-mutant expression vector, showed the converse specificity, inhibiting the triple mutant but not affecting the wild-type H5 pseudovirus (FIGS. 7, B and C, right). Finally, although 9E8 more effectively neutralized the wild type than S137A, T1921, another antibody, 11H12, showed comparable activity on both (FIG. 7D), confirming the differential antigenicity of this mutant. Modification of SA binding specificity therefore altered neutralization sensitivity and facilitated the generation of vaccines that elicited effective neutralizing mAbs.

In this report, we have identified mutations in the avian H5 hemagglutinin that alter its specificity for SA receptors and have shown that such mutants can be used to elicit neutralizing monoclonal antibodies that more effectively inhibit these variants. Neutralization sensitivity was determined with a lentiviral entry assay previously shown to define mechanisms of entry for numerous viruses, including HIV, severe acute respiratory syndrome (SARS), Ebola and Marburg hemorrhagic viruses, and, recently, influenza (Li, W. et al. 2003 Nature 426:450; Yang, Z. et al. 1998 Science 279:1034; Yang, Z.-Y. et al. 2004 J Virol 78:5642). Inhibition by antibodies determined neutralization sensitivity (Example 1; Kong, W.-P. et al. 2006 Proc Natl Acad Sci USA 103:15987) and correlated with hemagglutination inhibition, a traditional marker of immune protection (Table 7) (Kong, W.-P. et al. 2006 Proc Natl Acad Sci USA 103:15987). With this approach, the specificity of the HA was examined, independent of molecular adaptations required to generate replication-competent virus, which allowed identification of several mutants with altered SA specificity. Other mutants have been defined recently whose recognition was assessed with a less-specific assay (Yamada, S. et al. 2006 Nature 444:378), and we find here that they do not gain α2,6-SA recognition in the HA assay (Table 5B; N186K, Q196R). The previously reported Q226L, G228S mutant (Stevens, J. et al. 2006 Science 312:404) also showed no α2,6-SA binding (Table 5A). It is therefore unlikely that HA mutants reported previously are human-adapted, although S137A, T192I here may represent a step in this pathway.

Whether acquisition of α2,6-SA specificity would increase H5N1 transmissibility also remains unknown. Recently, HA mutations in the 1918 virus that allowed human SA recognition were shown to enhance transmission in ferrets (Tumpey, T. M. et al. 2007 Science 315:655), which supports this notion and provides a model to evaluate such H5 mutants. The approach to rational design of human-adapted H5-specific vaccines facilitates such analyses, as well as the development of preemptive countermeasures to contain influenza outbreaks. The five major antigenic sites of HA lie on an accessible surface adjacent to the RBD (Skehel, J. J. and Wiley, D.C. 2000 Annu Rev Biochem 69:531; Wiley, D. C. et al. 1981 Nature 289:373; Kaverin, N. V. et al. 2002 J Gen Virol 83:2497). Although antibodies to this region can affect RBD specificity and neutralization sensitivity (Skehel, J. J. and Wiley, D. C. 2000 Annu Rev Biochem 69:531, Laeeq, S. et al. 1997 J Virol 71:2600; Ilyushina, N. et al. 2004 Virology 329:33; Bizebarb, T. Et al. 1995 Nature 376:92; Fleury, D. et al. 1999 Nat Struct Biol 6:530), changes solely in the RBD have not been shown to alter immunogenicity. Here, structure-based modification of RBD specificity facilitated the generation of mAbs independent of the major antigenic sites. Directed to a functionally constrained domain, they may less readily evolve resistance and serve as vaccine prototypes that are envisioned as being developed before human-adapted strains emerge.

### Monoclonal Antibodies 9B11, 10D10, 9E8, and 11H12

After a long history of scientific study involving polyclonal antibodies, the development of a way to generate monoclonal antibodies in 1975 was, of course, an enormous technical leap. Monoclonals are invaluable for many tasks, including assaying for, characterizing and purifying their cognate antigens. Their exquisite specificity for their target made them obvious candidates for pharmaceutical use. However, the fact that hybridomas must be made in experimental animals rather than humans means that the monoclonal antibodies they produce have limited value as human therapeutics. An antibody derived from a mouse has a sequence that is recognized as foreign by a human immune system, and consequently raises a potent and potentially destructive immune response when administered to a human. Careful study of the structure of antibodies over the years led to marked improvements in this regard. In 1983, the concept of chimeric antibodies became a reality. In a chimeric antibody, the heavy and light chain variable regions of a mouse or other non-human (“donor”) monoclonal antibody are attached, using recombinant DNA technology, to the heavy and light chain constant region of a human antibody. This greatly reduces the antibody's potential immunogenicity in humans while preserving its specificity. The next technological breakthrough, “humanization”, came a few years later. In a “humanized” antibody, only the three CDRs (complementarity determining regions) and sometimes a few carefully selected “framework” residues (the non-CDR portions of the variable regions) from each donor antibody variable region are recombinantly pasted onto the corresponding frameworks and constant regions of a human antibody sequence. More recently the field has developed various ways to generate “fully human” antibodies: e.g., by creating hybridomas from mice genetically engineered to have only human-derived antibody genes, or by selection from a phage-display library of human-derived antibody genes. Yet another variant structure is a single-chain Fv, or “scFv”, in which a light chain variable region of a monoclonal antibody is recombinantly fused, through a linker sequence, to a heavy chain variable region of the antibody.

As used herein, “specific binding” refers to the property of the monoclonal antibody to bind the cognate antigen to which any of monoclonal antibody 9B11, 10D10, 9E8, or 11H12 binds with an affinity that is at least two-fold, 50-fold, 100-fold, 1000-fold, or more greater than its affinity for binding to a non-specific antigen (e.g., BSA, casein) other than said cognate antigen.

As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). Preferably, each VH and VL is composed of three CDRs and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system. The term “antibody” includes intact immunoglobulins of types IgA, IgG, IgE, IgD, IgM (as well as subtypes thereof), wherein the light chains of the immunoglobulin may be of types kappa or lambda.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids). The term “immunoglobulin” includes an immunoglobulin having: CDRs from a non-human source, e.g., from a non-human antibody, e.g., from a mouse immunoglobulin or another non-human immunoglobulin, from a consensus sequence, or from a sequence generated by phage display, or any other method of generating diversity; and having a framework that is less antigenic in a human than a non-human framework, e.g., in the case of CDRs from a non-human immunoglobulin, less antigenic than the non-human framework from which the non-human CDRs were taken. The framework of the immunoglobulin can be human, humanized non-human, e.g., a mouse, framework modified to decrease antigenicity in humans, or a synthetic framework, e.g., a consensus sequence. These are sometimes referred to herein as modified immunoglobulins. A modified antibody, or antigen binding fragment thereof, includes at least one, two, three or four modified immunoglobulin chains, e.g., at least one or two modified immunoglobulin light and/or at least one or two modified heavy chains. In one embodiment, the modified antibody is a tetramer of two modified heavy immunoglobulin chains and two modified light immunoglobulin chains.

As used herein, “isotype” refers to the antibody class (e.g., IgM or IgG1) that is encoded by heavy chain constant region genes.

The term “antigen-binding fragment” of an antibody (or simply “antibody portion,” or “fragment”), as used herein, refers to a portion of an antibody which specifically binds to the antigen of interest, e.g., a molecule in which one or more immunoglobulin chains is not full length but which specifically binds to the antigen of interest. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VL and VH, can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition.

The term “recombinant” antibody, as used herein, refers to antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant antibodies include humanized, CDR grafted, chimeric, in vitro generated (e.g., by phage display) antibodies, and may optionally include constant regions derived from human germline immunoglobulin sequences.

In a preferred embodiment, we provide a monospecific antibody (e.g., a monoclonal antibody) or an antigen-binding fragment thereof. The antibodies (e.g., recombinant or modified antibodies) can be full-length (e.g., an IgG (e.g., an IgG1, IgG2, IgG3, IgG4), IgM, IgA (e.g., IgA1, IgA2), IgD, and IgE, but preferably an IgG) or can include only an antigen-binding fragment (e.g., a Fab, F(ab′)2 or scFv fragment, or one or more CDRs). An antibody, or antigen-binding fragment thereof, can include two heavy chain immunoglobulins and two light chain immunoglobulins, or can be a single chain antibody. The antibodies can, optionally, include a constant region chosen from a kappa, lambda, alpha, gamma, delta, epsilon or a mu constant region gene. A preferred antibody includes a heavy and light chain constant region substantially from a human antibody, e.g., a human IgG1 constant region or a portion thereof. In some embodiments, the antibodies are human antibodies.

The antibody (or fragment thereof) can be a murine or a human antibody. Examples of preferred monoclonal antibodies that can be used include a 9B11, 10D10, 9E8, and 11H12 antibody. Also within the scope of the invention are methods and composition using antibodies, or antigen-binding fragments thereof, which bind overlapping epitopes of, or competitively inhibit, the binding of the antibodies disclosed herein to the cognate antigens, e.g., antibodies which bind overlapping epitopes of, or competitively inhibit, the binding of monoclonal antibodies 9B11, 10D10, 9E8, or 11H12 to the cognate antigens. Any combination of antibodies can be used, e.g., two or more antibodies that bind to different regions of the cognate antigens, e.g., antibodies that bind to two different epitopes on the cognate antigens.

In some embodiments, the antibody or an antigen-binding fragment binds to all or part of the epitope of an antibody described herein, e.g., a 9B11, 10D10, 9E8, and 11H12 antibody. The antibody can inhibit, e.g., competitively inhibit, the binding of an antibody described herein, e.g., a 9B11, 10D10, 9E8, and 11H12 antibody, to the cognate antigens. An antibody may bind to an epitope, e.g., a conformational or a linear epitope, which epitope when bound prevents binding of an antibody described herein, a 9B11, 10D10, 9E8, and 11H12 antibody. The epitope can be in close proximity spatially or functionally associated, e.g., an overlapping or adjacent epitope in linear sequence or conformationally to the one recognized by the 9B11, 10D10, 9E8, and 11H12 antibody.

In other embodiments, the antibodies (or fragments thereof) are a recombinant or modified antibody chosen from, e.g., a chimeric, a humanized, or an in vitro generated antibody. As discussed herein, the modified antibodies can be CDR-grafted, humanized, or more generally, antibodies having CDRs from a non-human antibody and a framework that is selected as less immunogenic in humans, e.g., less antigenic than the murine framework in which a murine CDR naturally occurs. In one embodiment, a modified antibody is a humanized form of 9B11, 10D10, 9E8, or 11H12 antibody.

In another aspect, the invention features a composition for use for preventing or treating an influenza virus infection. The composition includes a antibody or an antigen-binding fragment thereof as described herein. The composition of the invention can further include a pharmaceutically acceptable carrier, excipient or stabilizer.

The antibody or an antigen-binding fragment thereof as described herein can be administered to the subject systemically (e.g., intravenously, intramuscularly, by infusion, e.g., using an infusion device, subcutaneously, transdermally, or by inhalation). In those embodiments where the antibody or an antigen-binding fragment thereof is a small molecule, it can be administered orally. In other embodiment, the antibody or an antigen-binding fragment thereof is administered locally (e.g., topically) to an affected area, e.g., the respiratory tract.

The subject can be mammal, e.g., a primate, preferably a higher primate, e.g., a human (e.g., a patient having, or at risk of, an influenza virus infection).

In another aspect, the invention features methods for detecting the presence of the cognate antigen in a sample, in vitro (e.g., a biological sample, such as plasma, tissue biopsy). The subject method can be used to evaluate, e.g., diagnose or stage an influenza virus infection. The method includes: (i) contacting the sample (and optionally, a reference, e.g., a control sample) with a antibody or an antigen-binding fragment thereof under conditions that allow interaction of the antibody or fragment thereof and the cognate antigen to occur; and (ii) detecting formation of a complex between the antibody or an antigen-binding fragment thereof and the sample (and optionally, a reference, e.g., a control sample). Formation of the complex is indicative of the presence of the cognate antigen, and can indicate the suitability or need for a treatment described herein. For example, a statistically significant change in the formation of the complex in the sample relative to the control sample is indicative of the presence of the cognate antigen in the sample.

In yet another aspect, the invention provides a method for detecting the presence of the cognate antigen, in vivo (e.g., in vivo imaging in a subject). The subject method can be used to evaluate, e.g., diagnose or stage an influenza virus infection in a subject, e.g., a mammal, e.g., a primate, e.g., a human. The method includes: (i) administering to a subject (and optionally, a reference, e.g., a control subject) a antibody or an antigen-binding fragment thereof, under conditions that allow interaction of the antibody or fragment thereof and the cognate antigen to occur; and (ii) detecting formation of a complex between the antibody or an antigen-binding fragment thereof and the cognate antigen. A statistically significant change in the formation of the complex in the subject relative to the reference, e.g., the control subject or subject's baseline, is indicative of the presence of the cognate antigen.

Preferably, the antibody or an antigen-binding fragment thereof is directly or indirectly labeled with a detectable substance to facilitate detection of the bound or unbound binding agent. Suitable detectable substances include various biologically active enzymes, prosthetic groups, fluorescent materials, luminescent materials, paramagnetic (e.g., nuclear magnetic resonance active) materials, and radioactive materials. In some embodiments, the antibody or fragment thereof is coupled to a radioactive ion, e.g., indium (¹¹¹In), iodine (¹³¹I or ¹²⁵I), yttrium (⁹⁰Y), lutetium (¹⁷⁷Lu), actinium (²²⁵Ac), bismuth (²¹²Bi or ²¹³Bi), sulfur (³⁵S), carbon (¹⁴C), tritium (³H), rhodium (¹⁸⁸Rh), technetium (⁹⁹mTc), praseodymium, or phosphorous (³²P).

### Example 1

Genbank Accession Numbers used were AY651364, AY555150, DQ868374 and DQ868375.

### Immunogen and Plasmid Construction

Plasmids encoding the H5N1(KAN-1) (GenBank accession no. AY555150) hemagglutinin have been previously described (W.-P. Kong et al. 2006 Proc Natl Acad Sci USA 103:15987) and were synthesized using human-preferred codons (GeneArt, Regensburg, Germany). The sequences have been submitted to GenBank, accession no. DQ868374. The mutant HAs were prepared by site-directed mutagenesis using a QuickChange kit (Stratagene, La Jolla, Calif.) as indicated in the text. Protein expression was confirmed by Western blot analysis (W. P. Kong et al. 2003 J Virol 77:12764). The immunogens used in DNA vaccination contained a cleavage site mutation (PQRERRRKKRG (SEQ ID NO: 3) to PQRETRG (SEQ ID NO: 4)) as previously described (W.-P. Kong et al. 2006 Proc Natl Acad Sci USA 103:15987) (GenBank accession no. DQ868375). This modification is also denoted “mut.A”. Plasmids expressing the secreted trimeric form of HA and triple mutant HA(E190D/K193S/G225D) were generated by fusing amino acids 1-518 of HAs containing a cleavage site mutation as described above to LVPRGSPGSGYIPEAPRDGQAYVRKDGEWVLLSTFLGHHHHHH (SEQ ID NO: 5) as described (thrombin cleavage site in italics, external trimerization region in bold) (J. Stevens et al. 2006 Science 312:404). This modification is also denoted “short” and “foldon” because not only does it contain a trimerization site but also the fusion results in truncation of the HA protein at the carboxy terminus 10 amino acids upstream of the transmembrane domain. A plasmid encoding the N1(KAN-1) (GenBank accession no. AY555150) was also synthesized using human-preferred codons (GeneArt, Regensburg, Germany).

### Vaccination

Female BALB/c mice, 6-8 weeks old (Jackson Labs), were immunized as previously described (Z.-Y. Yang et al: 2004 Nature 428:561). Briefly, mice were immunized three times with 15 μg plasmid DNA in 100 μl of PBS (pH 7.4) intramuscularly at weeks 0, 3, 6 for DNA immunization alone, or for prime-boost vaccination to generate neutralizing monoclonal antibodies, followed by additional boosting with 10¹⁰ particles of recombinant adenovirus (rAd) expressing the same antigen at week 8-10. Serum was collected 10 days after the last vaccination. Ferrets were similarly immunized except using 200 μg plasmid DNA.

### Cell Lines, Antibodies, Lectins and Sialic Acid Analogues

Human embryonic kidney cell lines 293T, 293A, and 293F were purchased from Invitrogen (Carlsbad, Calif.) as a viral producer and as a target cell of infection, or for protein production respectively. They have been described previously (Z.-Y. Yang et al. 2004 J Virol 78:5642). Rabbit anti-HA(H5N1) IgG was purchased from Immune Technology (Queens, N.Y.). Rabbit anti-p24(HIV-1) antisera was obtained from ABI (Columbia, Md.). Maackia amurensis lectin II (MAA), Sambucus nigra lectin (SNA), biotinylated MAA or SNA, and FITC-labeled streptavidin came from Vector Laboratories (Burlingame, Calif.).

### Production of Anti-H5 Mouse Monoclonal Antibodies

Female BALB/c mice were immunized with plasmid DNA three times, followed by boosting with 10¹⁰ particles of rAd expressing the same antigen. Three days after boosting, spleens from the mice were harvested, homogenized into single cell suspensions, fused with Sp2/0-Ag14 myeloma as a partner using polyethylene glycol, and hybridomas were selected in an HAT-containing medium as previously described (G. Kohler and C. Milstein 1976 Eur J Immunol 6:511; S. N. Iyer et al. 1998 Hypertension 31:699) at Lofstrand Labs (Gaithersburg, Md.). Hybrids producing the antibody of interest were screened with ELISA, and pseudotype neutralization assays were performed as previously described (W.-P. Kong et al. 2006 Proc Natl Acad Sci USA 103:15987). Three clones that showed strong neutralization, 10D10, 9E8, and 9B11, were isolated, and they were subsequently adapted to serum-free medium. Another clone with neutralizing activity, 11H12, was isolated from a subsequent fusion and was also used to characterize the S137A,T192I mutant. Mouse monoclonal antibodies were purified from serum-free cell culture medium of each hybridoma using HiTrap protein G affinity columns (Amersham, Piscataway, N.J.).

### Production and Purification of Trimeric HA Protein

Plasmids expressing a secreted trimer of HA and HA(E190D/K193S/G225D) were transfected into 293F cells using 293fectin (Invitrogen™,Carlsbad, Calif.) with or without a tenth ratio of NA(KAN-1) expressing vector (weight: weight). 72-96 hrs after transfection, cell culture supernatant was collected, cleared by centrifugation, filtered, and purified using a Ni Sepharose™ High-performance affinity column (GE Healthcare, Piscataway, N.J.) as previously described (J. Stevens et al. 2006 Science 312:404). Fractions were combined and subjected to ion-exchange chromatography (mono-Q HR10/10, GE Healthcare, Piscataway, N.J.) and gel filtration chromatography (Hiload 16/60 Superdex 200 pg, GE Healthcare, Piscataway, N.J.). The fractions containing trimers were combined, and dialyzed against PBS.

### Surface Staining of HA and α2,3 and α2,6 Sialic Acids

293 T cells were co-transfected with plasmids expressing wild type and H5 mutants using Lipofectamine 2000 (Invitrogen, Carlsbad, Calif.). 24 hours after transfection, cells were removed using PBS with 2 mM EDTA, collected, and washed with PBS. Cells were stained with mouse anti-HA[H5N1(KAN-1)] sera (FIG. 5A; black line, 1:200) or a preimmune sera control (FIG. 5A, gray line, 1:200). Alternatively, cells co-transfected with NA(KAN-1), 0.1 w/w ratio, were incubated with monoclonal antibodies (9E8, 10D10, 9B11) (FIG. 7A; black line, 5 μg/ml or an isotype control (FIG. 7A, gray line, 5 μg/ml) for 30 minutes on ice, washed, and incubated with Alexa Fluor 488-goat anti-mouse IgG (Invitrogen, Carlsbad, Calif.) (1:2000) for 30 minutes on ice. Samples were washed and analyzed using a FACSCalibur Flow Cytometer (BD, Franklin Lakes, N.J.).

For surface staining of α2,3- and α2,6-SAs (FIG. 5B), 293A cells were collected and analyzed as described above. After incubation with biotinylated MAA (10 μg/ml) or biotinylated SNA (10 μg/ml) for 30 minutes on ice, the cells were washed and incubated with FITC-labeled streptavidin (10 μg/ml) for 30 minutes on ice.

### Production of Pseudotyped Lentiviral Vectors

The recombinant lentiviral vectors expressing a luciferase reporter gene were produced as previously described (L. Naldini et al. 1996 Proc Natl Acad Sci USA 93:11382). Briefly, 293T cells in a 10 cm dish were co-transfected with 400 ng of H5 HA or HA mutants, 50 ng of NA NA(H5N1/KAN-1) expression vector, 7 μg of pCMVΔR8.2, and 7 μg of pHR/CMV-Luc plasmid using a calcium phosphate transfection kit (Invitrogen, Carlsbad, Calif.) overnight, and replenished with fresh media. 48 hours later, supernatants were harvested, filtered through a 0.45 μm syringe filter, stored in aliquots, and used immediately or frozen at −80° C. The input viruses were standardized by the amount of p24 in the virus preparation. The p24 level was measured from different viral stocks using the HIV-1 p24 Antigen Assay kit (Beckman Coulter, Fullerton, Calif.). Analysis of HA expression in these preparations was confirmed after buoyant density centrifugation using Western blot analysis, and levels varied by no more than 1- to 2-fold.

Infection of Cells with Pseudotyped Lentiviral Vectors

A total of 30,000 293A cells were plated into each well of a 48-well dish one day prior to infection. Cells were incubated with 100 μl of viral supernatant/well in triplicate with HA NA-pseudotyped viruses for 14-16 hours. Viral supernatant was replaced with fresh media at the end of this time, and luciferase activity was measured 48 hours later as previously described (Z.-Y. Yang et al. 2004 J Virol 78:5642) using “mammalian cell lysis buffer” and “Luciferase assay reagent” (Promega, Madison, Wis.) according to the manufacturer's protocol.

### Inhibition of HA NA Pseudovirus Entry by Mouse Anti-serum and Monoclonal Antibodies

HA NA-pseudotyped lentiviral vectors encoding luciferase were first titrated by serial dilution. Similar amounts of viruses (p24 ≈6.25 ng/ml) were then incubated with indicated amounts of mouse antisera or monoclonal antibodies for 20 minutes at room temperature and added to 293A cells (10,000 cells/well in a 96-well-dish) (50 μl/well, in triplicate). Plates were washed and replaced with fresh media 6 hours later. Luciferase activity was measured after 24 hours.

### Glycan Array Analysis of Hemagglutinin

HA-antibody pre-complexes were prepared by mixing 15 μg HA and 7 μg Alexa Fluor488 labeled mouse anti-penta His (Qiagen, Cat# 1019199) at a molar ratio of 2:1 in a total volume of 50 μl and the mixtures were incubated for 15 min on ice. The pre-complex was then diluted with 50 microliter of PBS containing 3 percent (w/v) bovine serum albumin and 0.05 percent Tween 20. An aliquot of the diluted pre-complex was applied to the microarray (version 3.0) under a cover slip and incubated in a dark, humidified chamber for 1 hour at room temperature. The cover slip was gently removed and the slide subsequently washed by successive rinses in PBS with 0.05 percent Tween-20, PBS and deionized water. To remove excess water, the slide was spun in a slide microcentrifuge for 30 second, and binding image was read in a microarray scanner (ProScanArray, PerkinElmer). Image analysis was performed using Imagene v.6 software (BioDiscovery, El Segundo, Calif.), and results files are generated in Excel format where the Relative Fluorescence (RFU) from 6 replicates of each glycan (Table 8) was reported as the average of n=4 after elimination of the highest and lowest values. Data was uploaded to the Consortium for Functional Glycomics database on the world-wide-web at functionalglycomics.org/glycomics/publicdata/primaryscreen.jsp.

### Hemagglutination of H5N1 and Other Pseudoviruses to Measure Receptor Specificity

Hemagglutination of chicken RBC (CRBC) and enzymatically modified CRBC was done as previously described (L. Naldini et al. 1996 Proc Natl Acad Sci USA 93:11382; L. Glaser et al. 2005 J Virol 79:11533; T. G. Ksiazek et al. 2003 N Engl J Med 348:1953; J. C. Paulson and G. N. Rogers 1987 Methods Enzymol 138:162). To make SA α2,3Gal or α2,6Gal resialylated CRBC, 0.6 ml of 10% (v/v) freshly prepared CRBCs (Innovative Research, Southfield, Mich.) were washed three times with 10 ml PBS (pH 7.4), and treated with 200 mU vibrio cholerae neuraminidase (Roche, Indianapolis, Ind.) for 1 hour at 37° C. After three washes with 1 ml PBS, cells were resuspended in 1 ml PBS, incubated with 20 mU of α2,3(N)-sialyltransferase (Calbiochem, La Jolla, Calif.) for 30 min. at 37° C.; or in 1.5 ml PBS with 4.5 mU or α2,6(N)-sialyltransferase, kindly provided by Dr. James Paulson (Scripps Research Institute) for 45 min. at 37° C., plus 1.5 mM CMP-SA (Sigma, St. Louis, Mo.). The resialylated CRBCs were resuspended as 0.5% (v/v) in PBS after washing three times with PBS. Neuraminidase-treated CRBC were also incubated with pseudotyped viral vectors prior to resialation and uniformly showed titers of ≦1:2.

To measure the binding activity of pseudoviruses by hemagglutination, 50 ml of 1:5 diluted H5N1 pseudoviruses in PBS were added to 96 well round bottom plates, and serially diluted two-fold. 50 μl of 0.5% CRBC, α2,3, or α2,6 resialylated CRBC were added respectively, and mixed with viruses. HA titers were determined 60 minutes later by visual inspection.

TABLE 5
Specificity of glycan recognition and efficacy of entry
of wild-type and mutant HAs.

| | | HA titer | | | |
|---|---|---|---|---|---|
| Mutation | | CRBC | α2,3 | α2,6 | Entry |
| (A) | H5(KAN-1) | 80 | 160 | <2 | ++++ |
| | E190D | <2 | <2 | <2 | + |
| | G225D | 40 | <2 | <2 | ++++ |
| | E190, G225D | <2 | <2 | <2 | + |
| | Q226L | 40 | <2 | <2 | +++ |
| | Q226L, G228S | 40 | <2 | <2 | +++ |
| | E190D, K193S | 20 | <2 | <2 | +++ |
| | K193S, G225D | 80 | <2 | <2 | ++++ |
| | E190D, K193S, G225D | 40 | <2 | <2 | +++ |
| | K193S, Q226L | 20 | <2 | <2 | + |
| | K193S, Q226L, G228S | 40 | <2 | <2 | + |
| | H1N1(1918/SC) | 160 | <2 | 160 | ++++ |
| (B) | H5(VN1203) | 20 | 20 | <2 | ++++ |
| | E190D, K193S, Q226L, G228S | 40 | <2 | <2 | +++ |
| | A189K, K193N, Q226L, G228S | 40 | <2 | <2 | ++++ |
| | H5(VN1194) | 320 | 320 | <2 | ++++ |
| | N186K | 320 | 160 | <2 | ++++ |
| | Q196R | <2 | <2 | <2 | ++ |
| (C) | S137A | 80 | 80 | 80 | ++++ |
| | T192I | 80 | 160 | 80 | ++++ |
| | S137A/T192I | 40 | 40 | 80 | +++ |

H5 mutants KAN-1 from Thailand, or VN1203, and VN1194 from Vietnam were used as described in Example 1. The ability of indicated HAs to bind α2,3- and α2,6-SAs was determined by a resialylated hemagglutination assay (Example 1) for (A) KAN-1 mutants with loss of α2,3 HA activity and relevant controls, (B) VN1203 and previously described VN1194 mutants (Yamada, S. et al. 2006 Nature 444: 378), and (C) KAN-1 mutants with increased α2,6-SA binding. Viral entry of wild-type and mutant pseudotyped lentiviral vectors was measured as described (Example 1).
The degrees of entry were as follows:
+, <25% of WT;
++, 25 to 50% of WT;
+++, 50 to 75% of WT;
++++, >75% of WT.
The H5 (KAN-1) here is identical to the GenBank sequence and differs at amino acids 186(N/K) from Yamada and colleagues (Yamada, S. et al. 2006 Nature 444: 378), and the VN1194 mutants are identical to N182K and Q192R (Yamada, S. et al. 2006 Nature 444: 378) according to alternative numbering conventions.

TABLE 6
Summary of differences in glycan binding of S137A, T1921 compared to wild type by glycan microarray analysis.

| | | | | % DIFFERENCE |
|---|---|---|---|---|
| | | | M − C | RELATIVE TO |
| GLYCAN STRUCTURE | CONTROL | MUTANT | DIFFERENCE | CONTROL |
| Neu5Acβ2-6Galβ1-4GlcNAcβ-Sp8 | 39 | 186 | 147 | 378 |
| Neu5Acα2-6Galβ1-4[6OSO3]GlcNAcβ-Sp8 | 194 | 866 | 673 | 347 |
| Neu5Acα2-6Galβ1-4GlcNAcβ1-3Galβ1-4(Fucα1-3)GlcNAcβ1-3Galβ1-4 | 62 | 171 | 109 | 175 |
| (Fucα1-3)GlcNAcβ-Sp0 | | | | |
| Neu5Acα2-6Galβ1-4Glcβ-Sp0 | 17940 | 45263 | 27323 | 152 |
| Neu5Acα2-6Galβ1-4Glcβ-Sp8 | 37 | 92 | 55 | 147 |
| Neu5Acα2-6Galβ1-4GlcNAcβ1-2Manα1-3 | 16261 | 35011 | 18750 | 115 |
| (Neu5Acα2-3Galβ1-4GlcNAcβ1-2Manα1-6)Manβ1-4GlcNAcβ1-4GlcNAcb-Sp12 | | | | |
| Neu5Acα2-6Galβ1-4GlcNAcβ1-3Galβ1-4GlcNAcβ-Sp0 | 135 | 244 | 109 | 81 |
| Neu5Acα2-6Galβ1-4GlcNAcβ-Sp8 | 113 | 145 | 32 | 28 |
| Neu5Acα2-6Galβ1-4GlcNAcβ | 126 | 146 | 20 | 16 |
| Neu5Acβ2-6GalNAcα-Sp8 | 79 | 88 | 9 | 12 |
| Neu5Acα2-6Galβ-Sp8 | 95 | 71 | −24 | −26 |
| Galβ1-3(Neu5Acα2-3Galβ1-4(Fucα1-3)GlcNAcβ1-6)GalNAc-Sp14 | 404 | 21481 | 21077 | 5224 |
| Neu5Acα2-3Galβ1-3(Fucα1-4)GlcNAcβ-Sp8 | 820 | 30843 | 30023 | 3661 |
| NeuAcα2-3Galβ1-3(Fucα1-4)GlcNAcβ1-3Galβ1-4(Fucα1-3)GlcNAcβ Sp0 | 2351 | 47296 | 44945 | 1912 |
| Neu5Acα2-3Galβ1-4(Fucα1-3)GlcNAcβ1-3Galβ1-4(Fucα1-3) | 2248 | 25250 | 23003 | 1023 |
| GlcNAcβ1-3Galβ1-4(Fucα1-3)GlcNAcβ-Sp0 | | | | |
| Neu5Acα2-6GalNAcα-Sp8 | 144 | 76 | −68 | −47 |
| Neu5Acα2-6GalNAcβ1-4GlcNAcβ-Sp0 | 101 | 57 | −44 | −44 |
| Neu5Acα2-3Galβ1-3(Neu5Acα2-3Galβ1-4GlcNAcβ1-6)GalNAc-Sp14 | 62478 | 34367 | −28110 | −45 |
| Neu5Acα2-3Galβ1-4GlcNAcβ1-2Manα1-3 | 55128 | 37401 | −17727 | −32 |
| (Neu5Acα2-3Galβ1-4GlcNAcβ1-2Manα1-6)Manβ1-4GlcNAcβ1-4GlcNAcβ- | | | | |
| Sp12 | | | | |

The chemical structure, linkages, and binding of S137A, T192I relative to wt as determined by glycan microarray assays are shown. Glycan arrays were run as described in FIG. 6 so that binding was observed for most substrates in a linear range (50% maximal binding). A difference analysis was performed by subtracting the RFU of the S137A, T192I mutant from the RFU of the control. The difference was divided by the control and multiplied by 100 to obtain a percentage that represents positive or negative changes relative to the control data. Results are presented for three groups, including nine different structures containing Neu5Acα2-6Galβ1-4, seven of which showed a significant positive increase in binding by the mutant relative to the control, four compounds with fucose attached to polylactosamine, which showed substantially higher binding by S137A, T192I, and six α2-3 and α2-6 SAs that showed higher binding by the wt relative to S137A, T192I. Together, these analyses confirm the enhanced α2-6 recognition and altered RBD specificity of S137A, T192I relative to the wild type H5 KAN-1 HA.

TABLE 7
Neutralizing antibody responses of vaccinated animals determined by different assays.

| | | KAN-1 | | VN(1203) Hemagglutination | VN(1203) | KAN-1 Lentiviral |
|---|---|---|---|---|---|---|
| Animal | | Immunogen | Vector | Inhibition | Microneutralization | Inhibition (IC80) |
| Ferret | 1 | HA | 3xDNA + rAd | 40 | 40 | 420 |
| | 2 | HA | 3xDNA + rAd | 160 | 20 | 1557 |
| | 3 | HA | 3xDNA + rAd | ≧2560 | ≧2560 | 19879 |
| | 4 | HA | 3xDNA + rAd | 80 | 40 | 1251 |
| | 5 | HA | 3xDNA + rAd | ≧2560 | ≧2560 | 17186 |
| | 6 | HA | 3xDNA + rAd | 160 | 80 | 562 |
| | 7 | Control Vector | 3xDNA + rAd | 10 | 10 | 0 |
| | 8 | Control Vector | 3xDNA + rAd | 10 | 10 | 0 |
| Mouse | 1 | HA | Protein | 320 | ND | 1904 |
| | 2 | HA | Protein | 640 | ND | 1367 |
| | 3 | HA | Protein | 640 | ND | 3457 |
| | 4 | HA | 3xDNA + rAd | 640 | ND | 5401 |
| | 5 | HA | 3xDNA + rAd | ≧2560 | ND | 23518 |
| | 6 | HA | 3xDNA | 160 | ND | 172 |
| | 7 | HA | 3xDNA | 1280 | ND | 890 |
| | 8 | Control | 3xDNA | 160 | ND | 0 |
| Mab | 10D10 | HA | 3xDNA + rAd | 10 | ND | >6 μg/ml |
| | 9E8 | HA | 3xDNA + rAd | ≧2560 | ND | 0.2 μg/ml |

Sera from the indicated individual ferret or mouse groups immunized with H5 KAN-I HA encoded by DNA alone, DNA plus rAd (recombinant adenovirus) or purified KAN-I HA protein were evaluated by various methods. Hemagglutination inhibition and microneutralization assays were performed with rgA/Vietnam/1203/2004 x A/PR8/34 recombinant strain virus VN(1203) as previously described (J. J. Treanor et al. 2006 N Engl J Med 354: 1343) (Southern Research Institute, Birmingham, AL). End point dilutions are shown in the table. The lentiviral inhibition assay using A/Thailand/KAN-112004 HA lentiviral vector was performed as described in Example 1. Dilutions of the serum with IC80 activity are shown.
Mab refers to the mouse monoclonal antibodies described in FIG. 7.
IC80s of the monoclonal antibodies were calculated based on the purified IgG concentration.
ND represents samples not done.

TABLE 8
Chemical structure and designation of
glycans analyzed by microarray.

| | Carbohydrate | Dk97 | Viet04 |
|---|---|---|---|
| 1 | α₁-Acid Glycoprotein | ** | ** |
| 2 | α₁-Acid Glycoprotein A | ** | ** |
| 3 | α₁-Acid Glycoprotein B | nb | ** |
| 4 | Ceruloplasmine | nb | nb |
| 5 | Fibrinogen | nb | nb |
| 6 | Transferrin | nb | nb |
| 7 | 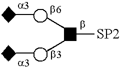 | ** | ** |
| 8 & 9 | 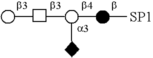 | nb | nb |
| 10 | 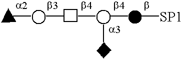 | nb | nb |
| 11 | 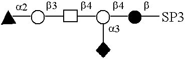 | nb | nb |
| 12 | 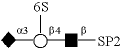 | nb | nb |
| 13 | 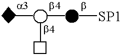 | nb | nb |
| 14 | 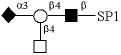 | nb | nb |
| 15 | 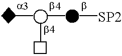 | nb | nb |
| 16 | 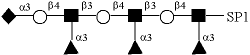 | nb | ** |
| 17 | 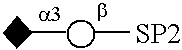 | nb | ** |
| 18 | 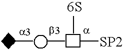 | ** | ** |
| 19 | 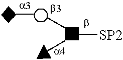 | nb | ** |
| 20 | 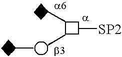 | ** | nb |
| 21 | 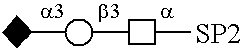 | * | ** |
| 22 | 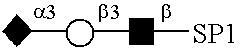 | nb | ** |
| 23 | 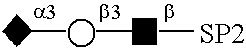 | * | ** |
| 24 | 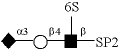 | * | ** |
| 25 | 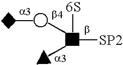 | * | ** |
| 26 | 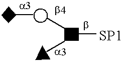 | nb | ** |
| 27 | 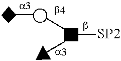 | nb | ** |
| 28 | 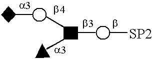 | nb | ** |
| 29 | 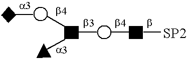 | nb | ** |
| 30 | 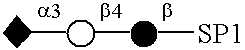 | nb | nb |
| 31 | 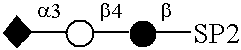 | nb | * |
| 32 | 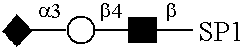 | nb | ** |
| 33 | 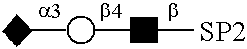 | ** | ** |
| 34 | 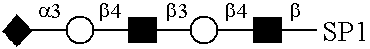 | nb | nb |
| 35 | 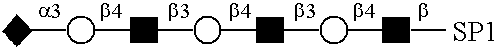 | * | ** |
| 36 | 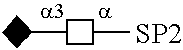 | nb | nb |
| 37 | 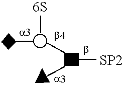 | nb | nb |
| 38 | 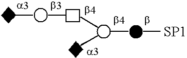 | ** | ** |
| 39 | 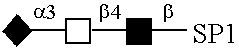 | nb | ** |
| 40 | 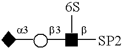 | ** | ** |
| 41 | 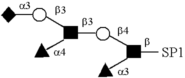 | nb | ** |
| 42 | 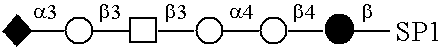 | nb | ** |
| 43 | 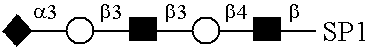 | nb | ** |
| 44 | 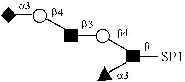 | nb | nb |
| 45 | 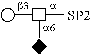 | nb | nb |
| 46 | 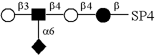 | nb | nb |
| 47 | 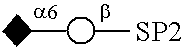 | nb | nb |
| 48 | 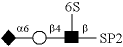 | nb | nb |
| 49 | 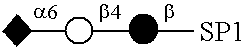 | nb | ** |
| 50 | 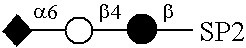 | nb | nb |
| 51 | 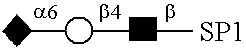 | nb | nb |
| 52 | 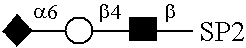 | nb | nb |
| 53 | 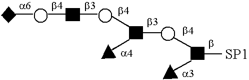 | nb | nb |
| 54 | 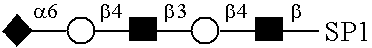 | nb | nb |
| 55 | 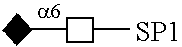 | nb | nb |
| 56 & 57 | 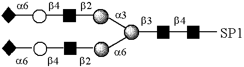 | nb | nb |
| 58 | 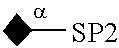 | nb | nb |
| 59 | 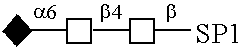 | nb | nb |
| 60 | 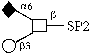 | nb | nb |
| 61 | 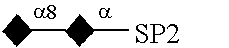 | nb | nb |
| 62 | 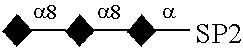 | nb | nb |
| 63 | 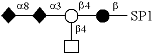 | nb | nb |
| 64 | 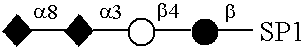 | nb | nb |
| 65 | 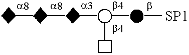 | nb | nb |
| 666 | 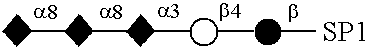 | nb | nb |
| 67 | 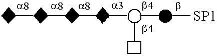 | nb | nb |
| 68 | 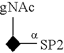 | nb | nb |
| 69 | 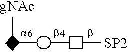 | nb | nb |
| 70 & 71 | 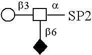 | nb | nb |
| 72 | 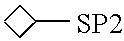 | nb | nb |
| 73 | 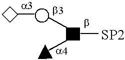 | nb | nb |
| 74 | 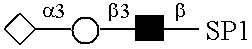 | nb | nb |
| 75 | 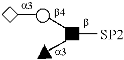 | nb | nb |
| 76 | 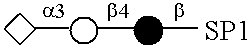 | nb | nb |
| 77 | 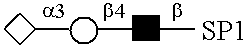 | nb | nb |
| 78 | 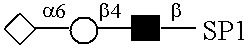 | nb | nb |
| 79 | 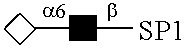 | nb | nb |
| 80 | 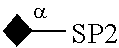 | nb | nb |
| 81 | 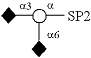 | nb | nb |
| 82 | 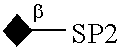 | nb | nb |
| 83 | 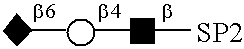 | nb | nb |
| 84 | 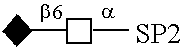 | nb | nb |

Chemical structure and linkages, name, and binding of indicated reference strains, as previously described (J. Stevens et al. 2006 Science 312: 404), are shown, providing a reference for the wt and triple mutant HAs shown in FIG. 6. Symbols are: white circle (Gal), black circle (Glc), black triangle (Fuc), white square (GalNAc), black square (GlcNAc), black diamond (Sialic acid), gray circle (Man), and white diamond (N-Glycolylsialic acid).

It is understood that the examples and embodiments described herein are for illustrative purposes only and that various modifications or changes in light thereof will be suggested to persons skilled in the art and are to be included within the spirit and purview of this application and scope of any appended claims. All figures, tables, and appendices, as well as publications, patents, and patent applications, cited herein are hereby incorporated by reference in their entirety for all purposes.

1. An isolated or recombinant hemagglutinin (HA) polypeptide, selected from the group consisting of:
(a) a polypeptide having at least 99.7% sequence identity to the amino acid sequence of SEQ ID NO:2;
(b) a polypeptide having at least 97% sequence identity to the amino acid sequence of SEQ ID NO: 82;
(c) a polypeptide having at least 97% sequence identity to the amino acid sequence of SEQ ID NO: 84;
(d) a polypeptide sequence comprising a fragment of (a), (b), or (c), the polypeptide comprising an amino acid sequence which is substantially identical over at least about 350 amino acids; over at least about 400 amino acids; over at least about 450 amino acids; or over at least about 500 amino acids contiguous of said (a), (b), or (c), wherein the fragment is immunogenic; and
(e) a H5 HA polypeptide;
wherein said polypeptide comprises a mutation at S137 to an amino acid other than 5, and, optionally, a further mutation at T192 to an amino acid other than T.

2. (canceled)

3. (canceled)

4. (canceled)

5. (canceled)

6. (canceled)

7. An antibody specific for the polypeptide of claim 1.

8. (canceled)

9. The polypeptide of claim 1, further comprising modification of the cleavage site to SEQ ID NO: 4.

10. The polypeptide of claim 1, further comprising modification of the carboxy terminus to the external trimerization region of SEQ ID NO: 5 in place of the transmembrane domain.

11. An isolated or recombinant nucleic acid comprising a polynucleotide encoding a hemagglutinin (HA) polypeptide, and which polynucleotide is selected from the group consisting of:
(a) a polynucleotide encoding a polypeptide having the amino acid sequence of SEQ ID NO:2, or a complementary polynucleotide sequence thereof;
(b) a polynucleotide encoding a polypeptide having the amino acid sequence of SEQ ID NO:82, or a complementary polynucleotide sequence thereof;
(c) a polynucleotide encoding a polypeptide having the amino acid sequence of SEQ ID NO:84, or a complementary polynucleotide sequence thereof;
(d) a polynucleotide encoding a polypeptide comprising a fragment of a polypeptide encoded by (a), (b), or (c), the polypeptide comprising an amino acid sequence which is substantially identical over at least about 350 amino acids; over at least about 400 amino acids; over at least about 450 amino acids; or over at least about 500 amino acids contiguous of said polypeptide encoded by (a), (b), or (c), wherein the polypeptide is immunogenic; and
a polynucleotide encoding a H5 HA polypeptide;
wherein said polynucleotide encodes a polypeptide comprising a mutation at 5137 to an amino acid other than S, and, optionally, a further mutation at T192 to an amino acid other than T.

12. The nucleic acid of claim 11, wherein the nucleic acid is DNA.

13. The nucleic acid of claim 11, wherein the nucleic acid is RNA.

14. (canceled)

15. An isolated or recombinant nucleic acid comprising a polynucleotide encoding a hemagglutinin (HA) polypeptide, and which polynucleotide has at least 95% identity to at least one polynucleotide of claim 11 (a), (b), or (c).

16. The nucleic acid of claim 11, wherein the polynucleotide encodes an immunogenic polypeptide.

17. (canceled)

18. (canceled)

19. (canceled)

20. (canceled)

21. (canceled)

22. (canceled)

23. A method for producing a hemagglutinin (HA) polypeptide in cell culture, the method comprising: introducing the nucleic acid of claim 14 into a host cell; culturing the host cell; and, recovering the hemagglutinin (HA) polypeptide.

24. (canceled)

25. (canceled)

26. (canceled)

27. (canceled)

28. A method of inducing an immune response to an influenza antigen in a subject, the method comprising: administering to the subject an immunogenic composition comprising the polypeptide of claim 1, in an amount effective to produce an immunogenic response against the influenza infection.

29. (canceled)

30. The method of claim 28, wherein the mammal is a human.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. A method of inducing an immune response to an influenza antigen comprising: administering to the subject an immunogenic composition comprising the nucleic acid of claim 11, in an amount effective to produce an immunogenic response against the influenza infection.
